(19) United States
(12) Patent Application Publication
(10) Pub. No.: US 2026/0125489 A1
(43) Pub. Date: May 7, 2026

(54) ANTI-LEWIS Y ANTIBODY AND PRO-ANTIBODY THEREOF

(71) Applicant: GLYCONEX INC., New Taipei City (TW)

(72) Inventors: Tong-Hsuan CHANG, Palos Verdes Estates, CA (US); Mei-Chun YANG, New Taipei City (TW); Liahng-Yim LIU, New Taipei City (TW); Yen-Ying CHEN, New Taipei City (TW); Chia-Chun CHANG, New Taipei City (TW); Wen-Han LEE, New Taipei City (TW); Yun-Chi LU, Taipei City (TW); Yi-An CHENG, Taipei City (TW)

(21) Appl. No.: 19/381,904

(22) Filed: Nov. 6, 2025

Related U.S. Application Data
(60) Provisional application No. 63/717,548, filed on Nov. 7, 2024.

(51) Int. Cl. C07K 16/30; A61K 45/06; A61K 47/68; A61P 35/00; C12N 15/11; C12N 15/85

(52) U.S. Cl. CPC C07K 16/30; A61K 45/06; A61K 47/68; A61P 35/00; C12N 15/11; C12N 15/85; C07K 2317/565; C07K 2317/732; C07K 2317/734; C07K 2317/77; C07K 2317/92

(57) ABSTRACT

The present disclosure relates to cancer therapies, in particular immunotherapies using antibodies or pro-antibodies to target Lewis Y antigens on tumor cells, thereby eliciting antibody-dependent cellular cytotoxicity (ADCC), complement-mediated cytotoxicity (CDC), and antibody-dependent cellular phagocytosis (ADCP).

### CROSS-REFERENCE TO RELATED APPLICATIONS

This application claims benefit of priority to U.S. Provisional Patent Application No. 63/717,548, filed on Nov. 7, 2024, the contents of which is hereby incorporated by reference in their entirety.

### REFERENCE TO SEQUENCE LISTING SUBMITTED AS A COMPLIANT XML FILE (.xml)

Pursuant to the EFS-Web legal framework and 37 C.F.R. § 1.821(c), a Sequence Listing is submitted herewith as an XML file named “3000179-001001_Sequence_Listing”, created on Nov. 6, 2025, and having a size of 47,755 bytes. The entire contents of the material in the aforementioned file is hereby incorporated by reference in its entirety.

### BACKGROUND

### Field

The present disclosure is related to cancer therapies, especially immunotherapies using antibodies to target tumor cells, thereby eliciting antibody-dependent cellular cytotoxicity (ADCC), complement-mediated cytotoxicity (CDC), or antibody-dependent cellular phagocytosis (ADCP).

Immunotherapies, using monoclonal antibodies (mAbs) for malignancies, infectious diseases, autoimmune diseases, transplant rejection, and chronic inflammatory diseases, have gradually become a mainstream therapeutic option due to their antigen specificity and longer half-life than conventional drugs. The clinical efficacy of monoclonal antibodies (mAbs) with antitumor activity has been demonstrated since the late 1990s (see, e.g., Topalian et al., J. Clin. Oncol. 29 (36): 4828-4836 (2011)), and several blockbuster cancer drugs in clinical uses are mAbs (e.g., rituximab, trastuzumab, and bevacizumab). Antibody-based therapies can specifically target antigen-expressing cells, such as tumor cells, and can affect cytotoxic activity through a number of means, including by eliciting antibody-dependent cellular cytotoxicity (ADCC), complement-mediated cytotoxicity (CDC), and antibody-dependent cellular phagocytosis (ADCP). Other approaches involve using antibodies as carrier vehicles to selectively deliver, e.g., cytotoxic or antiproliferative agents to kill target cells and/or to inhibit growth and metastatic spread of such cells. That is to say, antibody-based therapies have become an important tool for treating diseases in various aspects.

Glycan antigens, as components of glycoproteins and glycolipids expressed in the cell membrane, are getting increasing attention in cancer research. Lewis antigens are glycosyl antigens believed to mediate adhesion between cancer cells and endothelium via the selectin ligands thereof. It has also been proved that Lewis Y antigen (Lewis^{Y} or Le^{Y}) is overexpressed or misexpressed in a variety of cancers derived from epithelial tissues, including breast, lung, colon, ovarian cancers, etc, making Lewis Y antigen a potential target for antibody-based therapies.

### BRIEF SUMMARY

In one aspect, the present disclosure provides an isolated antibody or an antigen-binding fragment thereof, comprising: (a) a heavy chain variable region (VH) comprising a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3; and (b) a light chain variable region (VL) comprising a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

In one aspect, the present disclosure provides an isolated antibody or an antigen-binding fragment thereof comprising: a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15; a light chain variable region (VL) comprising an amino acid sequence having at least 90%, 91%, 92%, 93%, 94%, 95%, 96%, 97%, 98%, or 99% percent identity to SEQ ID NO: 11.

In one aspect, the present disclosure provides an isolated nucleic acid encoding the isolated antibody or the antigen-binding fragment thereof of the present disclosure.

In one aspect, the present disclosure provides a recombinant vector, comprising a nucleic acid encoding the isolated antibody or the antigen-binding fragment thereof of the present disclosure, or the nucleic acid of the present disclosure.

In one aspect, the present disclosure provides a recombinant vector, comprising a nucleic acid encoding a heavy chain variable region (VH) comprising: a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3.

In one aspect, the present disclosure provides a recombinant vector, comprising a nucleic acid encoding a light chain variable region (VL) comprising: a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

In one aspect, the present disclosure provides a pro-antibody, comprising an antigen-binding moiety configured to bind a Lewis Y antigen; and a masking moiety configured to inhibit the antibody or the antigen-binding fragment thereof from binding to the Lewis Y antigen; and wherein the masking moiety is linked to the antibody or the antigen-binding fragment thereof via a linker.

In one aspect, the present disclosure provides a pro-antibody, comprising an antigen-binding moiety and a masking moiety wherein the antigen-binding moiety and the masking moiety are linked via a cleavable linker, comprising a proteinase substrate flanked by a first linkage moiety and a second linkage moiety, and at least one of the first linkage moiety and the second linkage moiety comprises an amino acid sequence of PLAQ (SEQ ID NO: 37); wherein the antigen-binding moiety comprises (i) a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15; and a light chain variable region (VL) comprising an amino acid sequence having at least 90%, 91%, 92%, 93%, 94%, 95%, 96%, 97%, 98%, or 99% percent identity to SEQ ID NO: 11; or (ii) a VH comprising an amino acid sequence having at least 90%, 91%, 92%, 93%, 94%, 95%, 96%, 97%, 98%, or 99% percent identity to the amino acid sequence set forth in SEQ ID NO: 20; and a VL comprising an amino acid sequence having at least 90%, 91%, 92%, 93%, 94%, 95%, 96%, 97%, 98%, or 99% percent identity to the amino acid sequence set forth in SEQ ID NO: 25.

In one aspect, the present disclosure provides a pro-antibody, comprising a means for binding a Lewis Y antigen and a masking moiety, wherein the means for binding a Lewis Y antigen and the masking moiety are linked via a cleavable linker, comprising a proteinase substrate flanked by a first linkage moiety and a second linkage moiety, and at least one of the first linkage moiety and the second linkage moiety comprises an amino acid sequence of PLAQ (SEQ ID NO: 37).

In one aspect, the present disclosure provides an isolated nucleic acid encoding the pro-antibody of the present disclosure.

In one aspect, the present disclosure provides a recombinant vector, comprising the isolated nucleic acid encoding the pro-antibody of the present disclosure.

In one aspect, the present disclosure provides a host cell, comprising the recombinant vector comprising the isolated nucleic acid of the present disclosure, which encodes (1) the isolated antibody or the antigen-binding fragment thereof and/or (2) the pro-antibody of the present disclosure.

In one aspect, the present disclosure provides an antibody conjugate comprising a payload molecule covalently linked to the isolated antibody or the antigen-binding fragment thereof of the present disclosure or the antigen-binding moiety of the pro-antibody of the present disclosure.

In one aspect, the present disclosure provides a pharmaceutical composition, comprising (1) the isolated antibody or the antigen-binding fragment thereof, (2) the pro-antibody, and/or (3) the antibody conjugate of the present disclosure; and a pharmaceutically acceptable excipient.

In one aspect, the present disclosure provides a method of treating cancer, comprising administering (1) the isolated antibody or the antigen-binding fragment thereof, (2) the pro-antibody, (3) the antibody conjugate of the present disclosure, or (4) the pharmaceutical composition comprising the isolated antibody or the antigen-binding fragment thereof, the pro-antibody, and/or the antibody conjugate of the present disclosure to a subject in need thereof, wherein the payload molecule is a therapeutic agent.

In one aspect, the present disclosure provides a method of detecting cancer in a subject in need thereof, comprising administering the pharmaceutical composition comprising the antibody conjugate of the present disclosure to the subject in need thereof, wherein the payload molecule is a detectable indicator.

### BRIEF DESCRIPTION OF THE DRAWINGS

FIG. 1A is a graphic representation of a Glycan Antigen binding ELISA data showing the binding affinity of HKM1 and HKM4 to Lewis Y-Gal pentaose.

FIG. 1B is a graphic representation of a Glycan Antigen binding ELISA data showing the binding affinity of HKM1 and HKM4 to Lewis Y-tetraose.

FIG. 2 is a graphic representation of a Glycan Antigen binding ELISA data showing the binding affinity of HKM4 to Lewis Y-tetraose, Lewis Y-Gal pentaose, Lewis B-Gal pentaose, Lewis X, Lewis Y, and H type2.

FIG. 3 is a graphic representation of flow cytometry data showing the binding affinity of HKM4, BR96, and Isotype IgG to red blood cells.

FIG. 4 is a graphic representation of flow cytometry data showing the internalization of HKM4 upon binding a target cell.

FIG. 5 is a graphic representation showing data of a cancer cell binding assay. The assay tested the cancer cell binding abilities of antibody Ab12H1L and its humanized antibodies, HKM1 and HKM4, to cancer cell lines, AGS (human gastric adenocarcinoma), NCI-N87 (human gastric adenocarcinoma), DLD-1 (colorectal adenocarcinoma), and LS 174T (colorectal adenocarcinoma).

FIG. 6 is a graphic representation showing data of an anti-tumor activity assay. The Assay used an AGS xenograft tumor model to test the efficacy of the exemplary antibodies of the present disclosure in reducing tumor sizes. A conventional antibody, BR96, was also tested as a positive control.

FIG. 7 is a graphic representation showing data of a cancer cell binding assay, testing the design of pro-antibodies of the present disclosure. The pro-antibodies were tested before activation and after activation using MMP2.

FIG. 8 is a graphic representation of flow cytometry data showing the binding affinity of HKM4, pro-HKM4 (L-HKM4-L6S1), and Isotype IgG to red blood cells.

FIG. 9 shows images of an immunohistochemistry staining, showing that the L-HKM4-L6S1 only exhibited a low-intensity signal in two colorectal cancer patients' normal tissue adjacent (NAT) to the tumor.

FIG. 10A is a graphic representation showing data of an AGS cancer cell binding assay, testing HKM4 and L-HKM4 antibodies (before and after activation) of the present disclosure. The activation was conducted by using rhMMP2.

FIG. 10B is a graphic representation showing data of an LS 174T cancer cell binding assay, testing HKM4 and L-HKM4 antibodies (before and after activation) of the present disclosure. The activation was conducted by using rhMMP2.

FIG. 10C is a graphic representation showing data of an AGS cancer cell binding assay, testing BR96 and L-BR96 antibodies (before and after activation) of the present disclosure. The activation was conducted by using rhMMP2.

FIG. 10D is a graphic representation showing data of an LS 174T cancer cell binding assay, testing BR96 and L-BR96 antibodies (before and after activation) of the present disclosure. The activation was conducted by using rhMMP2.

FIG. 11A is a graphic representation showing data of an anti-tumor activity assay. The Assay used an AGS xenograft tumor model to test the efficacy of BR96 and L-BR96 antibodies in reducing tumor sizes.

FIG. 11B is a graphic representation showing data of an anti-tumor activity assay. The Assay used an AGS xenograft tumor model to test the efficacy of HKM4, L-HKM4-L1S1, and L-HKM4-L6S1 antibodies in reducing tumor sizes.

FIG. 12 is a graphic representation showing data of an anti-tumor activity assay. The Assay used an AGS xenograft tumor model to test the efficacy of the L-HKM4-L6S1 antibody and the antibody-drug conjugate of L-HKM4-L6S1 antibody and SN38.

FIG. 13 is a graphic representation showing the results of an AGS cell-binding assay. Samples (antibodies and pro-antibodies), with or without activation, were cultured with the AGS cells. The binding of the sample antibodies to the cells was determined and presented as MFI values using the formula: (L-HKM4-L6S1/L-HKM4-LO7)*100%. That is to say, the binding of L-HKM4-L6S1 was set as 100% to compare with the binding of L-HKM4-LO7 to the AGS cells. The result showed that L-HKM4-LO7's binding, before activation, was 160% at a concentration of 17.03 nM, 173% at 68.12 nM, and 146% at 272.48 nM.

FIG. 14 is a graphic representation showing the activation rate of exemplary pro-antibody L-HKM4-L6S1 and pro-antibody L-HKM4-LO7 in different patients' tissue samples. Activation ratio (%) of a pro-antibody was calculated by comparing the ROI of the pro-antibody and the ROI of the same antibody without the masking moiety: pro-antibody ROI/antibody ROI×100%). Note that the activation rates of the groups with protease inhibitors (PI) were very low, so they are not visible in this bar chart.

FIG. 15 is a graphic representation showing data of an anti-tumor activity assay. The Assay used an AGS xenograft tumor model to test the efficacy of HKM4, L-HKM4-L6S1, and L-HKM4-LO7 antibodies in reducing tumor sizes.

### DETAILED DESCRIPTION

Lewis antigens, unlike antigens of other blood group systems, are synthesized in the intestinal epithelial cells and circulate in plasma while being passively absorbed onto red blood cells. Lewis Y antigen is a terminally (at the non-reducing terminus) fucosylated carbohydrate epitope having a structure of Fucα1->2Galβ1→4 [Fucα1→3] GlcNAcβ1→R. Overexpression of Lewis Y antigen has been widely observed in a variety of cancers derived from epithelial tissues, including breast, lung, colon, ovarian cancers, etc. Overexpression of Lewis Y antigen is also believed to be associated with poor prognosis of cancer treatment. Given the potential therapeutic value, several anti-Lewis Y antibodies have been developed in this art. However, the conventional anti-Lewis Y antibodies reportedly cause gastrointestinal and hematological toxicities because of off-target binding to gastrointestinal tissue and red blood cells. This undesigned binding also results in reduced serum half-life of the antibody. There is, therefore, a need to develop an anti-Lewis Y antibody with desired binding specificity to Lewis Y antigen while having reduced non-specific binding or off-target binding, especially to gastrointestinal tissue and red blood cells, or an anti-Lewis Y antibody conjugate having a mechanism to avoid non-specific binding.

### Anti-Lewis Y Antibody

One aspect of the present disclosure discloses an isolated antibody or an antigen-binding fragment thereof configured to target a Lewis Y antigen, including a Lewis Y tetraose antigen or a Lewis Y pentaose antigen. In some embodiments, the antibody or the antigen-binding fragment thereof is configured to target a Lewis Y antigen or specifically bind a Lewis Y antigen at a dissociation constant (K_{D}) less than or equal to 10⁻³M, 10⁻⁴ M, 10⁻⁵ M, 10⁻⁶ M, 10⁻⁷ M, or 10⁻⁸ M, or any ranges defined by the aforesaid two endpoints, such as 10⁻³ M to 10⁻⁸ M, 10⁻³ M to 10⁻⁷ M, 10⁻³ M to 10⁻⁶ M, 10⁻³ M to 10⁻⁵ M, 10⁻³ M to 10⁻⁴ M, 10⁻⁴ M to 10⁻⁸ M, 10⁻⁴ M to 10⁻⁷ M, 10⁻⁴ M to 10⁻⁶ M, 10⁻⁴ M to 10⁻⁵ M, 10⁻⁵ M to 10⁻⁸ M, 10⁻⁵ M to 10⁻⁷ M, 10⁻⁵ M to 10⁻⁶ M, 10⁻⁶ M to 10⁻⁸ M, or 10⁻⁶ M to 10⁻⁷ M. In some embodiments, the K_{D} is about 10⁻⁶ M to 10⁻⁸ M.

Without wishing to be bound by theory, given the binding affinity to Lewis Y antigens, the antibody or the antigen-binding fragment is able to bind to a cancer cell expressing (e.g., overexpressing or mis-expressing) Lewis Y antigens and imposes effector functions, including but not limited to antibody-dependent cell-mediated cytotoxicity (ADCC), complement-mediated cytotoxicity (CDC), and antibody-dependent cellular phagocytosis (ADCP), phagocytosis.

In certain embodiments, the isolated antibody or the antigen-binding fragment thereof does not have cross-reactivity to Lewis X (LeX), Lewis A (LeA), Lewis B (LeB), or H type 2 antigen (e.g., the isolated antibody or the antigen-binding fragment thereof does not bind to those antigens), which is different from conventional anti-Lewis Y antibodies, such as BR96, which shows binding affinity to Lewis X (Zhang S, et al. Selection of tumor antigens as targets for immune attack using immunohistochemistry: II. Blood group-related antigens. Int J Cancer. 1997 Sep. 26; 73 (1): 50-6). Furthermore, in some embodiments, the antibody or the antigen-binding fragment thereof of the present disclosure possesses a reduced binding affinity to red blood cells. The antibody or the antigen-binding fragment thereof of the present disclosure differs from conventional anti-Lewis Y antibodies in that aspect, as the conventional anti-Lewis Y antibodies reportedly have the problem of causing gastrointestinal and hematological toxicities. In certain embodiments, the antibody of the antigen-binding fragment thereof of the present disclosure has a binding affinity to red blood cells that is 80%, 75%, 70%, 65%, or 60% of BR96's binding affinity to red blood cells, or any range defined by the foregoing two endpoints, such as 80% to 60%, 75% to 60%, 70% to 60%, 65% to 60%, 80% to 65%, 75% to 65%, or 70% to 65%; or the binding affinity to red blood cells is at least 20%, at least 25%, at least 30%, at least 35%, at least 40% lower than that of BR96. In some embodiments, the binding affinity to red blood cells can be determined by using flow cytometry and using an isotope IgG for normalization.

In some embodiments, the present disclosure also provides an isolated antibody or an antigen-binding fragment thereof that competes binding Lewis Y antigen with a reference antibody or an antigen-binding fragment thereof, wherein the reference antibody or an antigen-binding fragment thereof comprises the CDRs, the heavy chain variable region, the light chain variable region, the heavy chain, or the light chain of the present disclosure.

The isolated antibody or the antigen-binding fragment thereof of the present disclosure can be monoclonal. In some embodiments, the isolated antibody or the antigen-binding fragment thereof is a human antibody or a chimeric antibody, including a primatized antibody and a humanized antibody. In some embodiments, the antigen-binding fragment is selected from a group consisting of a Fab fragment, an F(ab′)2 fragment, an Fv fragment, a single-chain Fv (ScFv) antibody, a diabody, a minibody, a nanobody (VHH), and a linear antibody.

### CDRs

In some embodiments, the isolated antibody or the antigen-binding fragment thereof comprises a heavy chain variable region (VH) comprising a heavy chain complementarity determining region 1 (VH CDR1), a heavy chain complementarity determining region 2 (VH CDR2), and a heavy chain complementarity determining region 3 (VH CDR3). The VH CDR1 comprises an amino acid sequence that has at least 90%, 91%, 92%, 93%, 94%, 95%, 96%, 97%, 98%, or 99% percent identity to SEQ ID NO: 1, the VH CDR2 comprises an amino acid sequence that has at least 90%, 91%, 92%, 93%, 94%, 95%, 96%, 97%, 98%, or 99% percent identity to SEQ ID NO: 2, and/or the VH CDR3 comprises an amino acid sequence that has at least 90%, 91%, 92%, 93%, 94%, 95%, 96%, 97%, 98%, or 99% percent identity to SEQ ID NO: 3. In certain embodiments, the VH CDR1 comprises an amino acid sequence as set forth in SEQ ID NO: 1, while the VH CDR2 comprises an amino acid sequence that has at least 95%, 96%, 97%, 98%, or 99% percent identity to SEQ ID NO: 2, and the VH CDR3 comprises an amino acid sequence that has at least 95%, 96%, 97%, 98%, or 99% percent identity to SEQ ID NO: 3. In yet certain embodiments, the VH CDR1 comprises SEQ ID NO: 1, the VH CDR2 SEQ ID NO: 2, and the VH CDR3 comprises SEQ ID NO: 3.

In some embodiments, the isolated antibody or the antigen-binding fragment thereof further comprises a light chain variable region (VL) comprising a light chain complementarity determining region 1 (VL CDR1), a light chain complementarity determining region 2 (VL CDR2), and a light chain complementarity determining region 3 (VL CDR3). The VL CDR 1 comprises an amino acid sequence that has at least 90%, 91%, 92%, 93%, 94%, 95%, 96%, 97%, 98%, or 99% percent identity to SEQ ID NO: 8, the VL CDR2 comprises an amino acid sequence that has at least 90%, 91%, 92%, 93%, 94%, 95%, 96%, 97%, 98%, or 99% percent identity to SEQ ID NO: 9, and/or the VL CDR3 comprises an amino acid sequence that has at least 90%, 91%, 92%, 93%, 94%, 95%, 96%, 97%, 98%, or 99% percent identity to SEQ ID NO: 10. In certain embodiments, the VL CDR2 comprises an amino acid sequence as set forth in SEQ ID NO: 9, while the VL CDR1 comprises an amino acid sequence that has at least 95%, 96%, 97%, 98%, or 99% percent identity to SEQ ID NO: 8, and the VL CDR3 comprises an amino acid sequence that has at least 95%, 96%, 97%, 98%, or 99% percent identity to SEQ ID NO: 10. In yet certain embodiments, the VL CDR1 comprises SEQ ID NO: 8, the VL CDR2 comprises SEQ ID NO: 9, and the VL CDR3 comprises SEQ ID NO: 10.

### Heavy Chain and Light Chain Variable Region

In some embodiments, the isolated antibody or the antigen-binding fragment thereof of the present disclosure comprises a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15. In certain embodiments, the VH comprises an amino acid sequence set forth in SEQ ID NO: 4 or SEQ ID NO: 15. In certain embodiments, the VH is encoded by a sequence, having at least 70%, 75%, 80%, 85%, 90%, 91%, 92%, 93%, 94%, 95%, 96%, 97%, 98%, or 99% percent identity to SEQ ID NO: 6. In such embodiments, the isolated antibody or the antigen-binding fragment thereof comprises a VH CDR1, a VH CDR2, and a VH CDR3, as described herein or as defined according to Kabat, IMGT, Chothia, or EU numbering schemes.

In some embodiments, the isolated antibody or the antigen-binding fragment thereof further comprises a light chain variable region (VL) comprising an amino acid sequence having at least 90%, 91%, 92%, 93%, 94%, 95%, 96%, 97%, 98%, or 99% percent identity to SEQ ID NO: 11. In certain embodiments, the VL comprises an amino acid sequence set forth in SEQ ID NO: 11. In certain embodiments, the VL is encoded by a sequence, having at least 70%, 75%, 80%, 85%, 90%, 91%, 92%, 93%, 94%, 95%, 96%, 97%, 98%, or 99% percent identity to SEQ ID NO: 13. In such embodiments, the isolated antibody or the antigen-binding fragment thereof comprises a VL CDR1, a VL CDR2, and a VL CDR3, as described herein or as defined according to Kabat, IMGT, Chothia, or EU numbering schemes.

### Heavy Chain and Light Chain

In some embodiments, the isolated antibody or the antigen-binding fragment thereof comprises a heavy chain comprising an amino acid sequence having at least 90%, 91%, 92%, 93%, 94%, 95%, 96%, 97%, 98%, or 99% percent identity to SEQ ID NO: 5. In certain embodiments, the heavy chain comprises an amino acid sequence set forth in SEQ ID NO: 5. In certain embodiments, the heavy chain is encoded by a sequence, having at least 70%, 75%, 80%, 85%, 90%, 91%, 92%, 93%, 94%, 95%, 96%, 97%, 98%, or 99% percent identity to SEQ ID NO: 7. In such embodiments, the isolated antibody or the antigen-binding fragment thereof comprises a VH CDR1, a VH CDR2, and a VH CDR3, as described herein or according to Kabat, IMGT, Chothia, or EU numbering schemes.

In some embodiments, the isolated antibody or the antigen-binding fragment thereof further comprises a light chain comprising an amino acid sequence having at least 90%, 91%, 92%, 93%, 94%, 95%, 96%, 97%, 98%, or 99% percent identity to SEQ ID NO: 12. In certain embodiments, the light chain comprises an amino acid sequence set forth in SEQ ID NO: 12. In certain embodiments, the light chain is encoded by a sequence, having at least 70%, 75%, 80%, 85%, 90%, 91%, 92%, 93%, 94%, 95%, 96%, 97%, 98%, or 99% percent identity to SEQ ID NO: 14. In such embodiments, the isolated antibody or the antigen-binding fragment thereof comprises a VL CDR1, a VL CDR2, and a VL CDR3, as described herein or according to Kabat, IMGT, Chothia, or EU numbering schemes.

### Sequence Modification and Percent Identity

Two sequences are said to be “identical” if they contain the same residues at corresponding positions when aligned for maximum correspondence, as described herein. Comparisons between two sequences are typically performed by aligning the sequences to identify regions of optimal local or global similarity. A “comparison region” or “comparison window,” as used herein, refers to a segment of at least about 20 contiguous positions, typically about 30 to about 75, or about 40 to about 50 positions, in which one sequence is aligned to a reference sequence of the same number of contiguous positions after optimal alignment has been achieved. When the sequence being compared is shorter than the stated comparison region, the comparison is made over the entire length of the sequence.

In certain embodiments, “percent identity” is determined by aligning two sequences to obtain the highest degree of correspondence using a suitable sequence alignment algorithm (for example, the Needleman-Wunsch or Smith-Waterman algorithm). The “percent identity” is calculated as the number of identical residues (nucleotides or amino acids) shared between the two sequences within the aligned region divided by the total number of aligned positions, and multiplied by 100. Insertions or deletions (gaps) may be introduced in one or both sequences to optimize alignment; however, such gaps are generally minimized by use of appropriate gap penalties. Unless otherwise specified, the reference sequence is the longer or full-length sequence being compared.

In some embodiments, the modification described herein is also contemplated according to certain embodiments of the present disclosure. The modification can include conservative and non-conservative amino acid substitutions. A “conservative substitution” refers to amino acid substitutions that do not significantly affect or alter a particular characteristic (e.g., a binding activity such as a specific binding activity) of a particular protein. Generally, conservative substitutions are ones in which a substituted amino acid residue is replaced with an amino acid residue having a similar side chain. Exemplary conservative substitutions include a substitution found in one of the following groups:

| | |
|---|---|
| Group 1 | Alanine (Ala or A), Glycine (Gly or G), Serine (Ser or S), |
| | Threonine (Thr or T) |
| Group 2 | Aspartic acid (Asp or D), Glutamic acid (Glu or Z) |
| Group 3 | Asparagine (Asn or N), Glutamine (Gln or Q) |
| Group 4 | Arginine (Arg or R), Lysine (Lys or K), Histidine (His or H) |
| Group 5 | Isoleucine (Ile or I), Leucine (Leu or L), Methionine |
| | (Met or M), Valine (Val or V) |
| Group 6 | Phenylalanine (Phe or F), Tyrosine (Tyr or Y), |
| | Tryptophan (Trp or W) |

Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitution groups include sulfur-containing: Met and Cysteine (Cys or C); acidic: Asp, Glu, Asn, and Gin; small aliphatic, nonpolar or slightly polar residues: Ala, Ser, Thr, Pro, and Gly; polar, negatively charged residues and their amides: Asp, Asn, Glu, and Gin; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

Improving the binding affinity and/or other biological properties of an antibody or an antigen-binding fragment thereof according to one or more embodiments of the present disclosure may also be desirable. Amino acid sequence variants may be prepared, for instance, by introducing appropriate nucleotide changes into a nucleic acid that encodes the desired peptide or by peptide synthesis. Such modifications include, for example, deletions from, and/or insertions into, and/or substitutions of, residues within the amino acid sequence of the antibody or antigen-binding fragment thereof. Any combination of deletion, insertion, and substitution may be made to arrive at the final antibody or an antigen-binding fragment thereof, provided that the final construct possesses the desired characteristics (e.g., specific binding to a Lewis Y antigen, or lower binding affinity to red blood cells, not binding to a Lewis X antigen, as described herein). The amino acid changes also may alter post-translational processes of the antibody or antigen-binding fragment thereof, such as changing the number or position of glycosylation sites.

As described elsewhere herein, determination of the three-dimensional structures of the presently disclosed antibodies or antigen-binding fragments thereof may be made through routine methodologies such that substitution, addition, deletion, or insertion of one or more amino acids with selected natural or non-natural amino acids can be virtually modeled for purposes of determining whether a so derived structural variant retains the space-filling properties of presently disclosed species. A variety of computer programs are known to the skilled artisan for determining appropriate amino acid substitutions (or appropriate nucleic acids encoding the amino acid sequence) within an antibody or antigen-binding fragment thereof such that, for example, affinity is maintained or better affinity is achieved. See, for instance, Donate et al., 1994 Prot. Sci. 3:2378; Bradley et al., Science 309:1868-1871 (2005); Schueler-Furman et al., Science 310:638 (2005); Dietz et al., Proc. Nat. Acad. Sci. USA 103:1244 (2006); Dodson et al., Nature 450:176 (2007); Qian et al., Nature 450:259 (2007); Raman et al. Science 327:1014-1018 (2010); Marcos et al., 2017 Science 355:201, and references cited therein.

In some embodiments, the percent identity described herein reflects amino acid sequence modification(s) or nucleotide sequence modification(s) of a referenced sequence. A modification is acceptable, in certain embodiments, if the resulting antibody or the antigen-binding fragment thereof possesses the desired biologic characteristics, e.g., a desired binding affinity to a Lewis Y antigen, a reduced binding affinity to red blood cells, and/or not binding to a Lewis X antigen, as defined and described herein.

In some embodiments, a modification in the CDR region is acceptable if the 4th amino acid of the VH CDR1 is maintained as Isoleucine (I), the 5^{th} amino acid of the VH CDR2 is maintained as Aspartic acid (D), the 12^{th} amino acid of the VH CDR2 is maintained as Glutamine (Q), the 15^{th} amino acid of the VH CDR2 is maintained as Leucine (L), the 4^{th} amino acid of the VH CDR3 is maintained as Serine(S), the 5^{th} amino acid of the VH CDR3 is maintained as Glutamic acid (E), the 5^{th} amino acid of the VL CDR1 is maintained as Asparagine (N), and/or the 9^{th} amino acid of the VL CDR1 is maintained as Threonine (T).

In some embodiments, a modification in the CDR region is acceptable if the 4th amino acid of the VH CDR1 is not Methionine (M); the 5^{th} amino acid of the VH CDR2 is not Glycine (G) or Valine (V); the 12^{th} amino acid of the VH CDR2 is not Proline (P); the 15^{th} amino acid of the VH CDR2 is not Valine (V), the 4^{th} amino acid of the VH CDR3 is not Aspartic acid (D) or Arginine (R), the 5^{th} amino acid of the VH CDR3 is not Aspartic acid (D), the 5^{th} amino acid of the VL CDR1 is not (I) or Arginine (R), and/or the 9^{th} amino acid of the VL CDR1 is not Asparagine (N) or Serine(S).

In some embodiments, the percent identity describing a VH or VL sequence does not include a modification in a CDR region thereof. For example, in some embodiments, the VH comprises an amino acid sequence having at least 90%, 91%, 92%, 93%, 94%, 95%, 96%, 97%, 98%, or 99% percent identity to SEQ ID NO: 4 or SEQ ID NO: 15, provided that the percent identity reflects a modification residing in a non-CDR region or a framework (FW) region of the VH. Likewise, in some embodiments, the VL comprises an amino acid sequence having at least 90%, 91%, 92%, 93%, 94%, 95%, 96%, 97%, 98%, or 99% percent identity to SEQ ID NO: 11, provided that the percent identity reflects a modification residing in a non-CDR region or a framework (FW) region of the VL. However, the present disclosure is not so limited as, in some other embodiments, the percent identity describing a VH or VL sequence includes a modification in a CDR region thereof.

In some embodiments, a modification of the VH is acceptable if the 3^{rd} amino acid of the VH is maintained as Glutamine (Q), the 19^{th} amino acid of the VH is maintained as Arginine (R), the 20^{th} amino acid of the VH is maintained as Leucine (L), the 23^{rd} amino acid of the VH is maintained as Alanine (A), the 40^{th} amino acid of the VH is maintained as Alanine (A), the 84^{th} amino acid of the VH is maintained as Asparagine (N), the 85^{th} amino acid of the VH is maintained as Serine(S), the 87^{th} amino acid of the VH is maintained as Arginine (R), the 88th amino acid of the VH is maintained as Alanine (A), and/or the 93^{rd} amino acid of the VH is maintained as Valine (V).

In some embodiments, a modification of the VH is acceptable if the 3^{rd} amino acid of the VH is not Asparagine (N), the 19^{th} amino acid of the VH is not (K), the 20^{th} amino acid of the VH is not Valine (V), the 23^{rd} amino acid of the VH is not Valine (V) or Serine(S), the 40th amino acid of the VH is not (T), the 84^{th} amino acid of the VH is Serine(S) or Aspartic acid (D), the 85^{th} amino acid of the VH is Arginine (R), the 87^{th} amino acid of the VH is not Lysine (K), the 88^{th} amino acid of the VH is not Serine(S) or Proline (P), and/or the 93^{rd} amino acid of the VH is not Methionine (M).

In some embodiments, a modification of the VL is acceptable if the 2^{nd} amino acid of the VL is maintained as Isoleucine (I), the 3^{rd} amino acid of the VL is maintained as Valine (V), the 7^{th} amino acid of the VL is maintained as Threonine (T), the 14^{th} amino acid of the VL is maintained as Threonine (T), the 17^{th} amino acid of the VL is maintained as Glutamine (Q), the 18^{th} amino acid of the VL is maintained as Proline (P), the 88^{th} amino acid of the VL is maintained as Valine (V), the 105^{th} amino acid of the VL is maintained as Proline (P), the 109th amino acid of the VL is maintained as Valine (V), and/or the 110^{th} amino acid of the VL is maintained as Aspartic acid (D).

In some embodiments, a modification of the VL is acceptable if the 2^{nd} amino acid of the VL is not Valine (V), the 3^{rd} amino acid of the VL is not (L) or Glutamine (Q), the 7th amino acid of the VL is Isoleucine (I) or Serine(S), the 14^{th} amino acid of the VL is not Serine(S), the 17^{th} amino acid of the VL is not Aspartic acid (D), the 18^{th} amino acid of the VL is not Glutamine (Q) or (R), the 88^{th} amino acid of the VL is not Leucine (L) or Isoleucine (I), the 105th amino acid of the VL is not Serine(S) or Glutamine (Q), the 109^{th} amino acid of the VL is not Leucine (L), and/or the 110^{th} amino acid of the VL is not Glutamic acid (E) or Glutamine (Q).

On the other hand, a nucleotide modification is acceptable if the resulting nucleotide variant encodes an antibody or an antigen-binding fragment thereof, which possesses the desired characteristics, e.g., a desired binding affinity to a Lewis Y antigen, a reduced binding affinity to red blood cells, and/or not binding to a Lewis X antigen, as defined or described herein. Besides, it would be appreciated by a skilled artisan that, as a result of the degeneracy of the genetic code, many different nucleotide sequences might encode the same antibody or the antigen-binding fragment thereof of the present disclosure, such as those described herein. Some such polynucleotides bear minimal sequence identity to the nucleotide sequence of a native or original nucleotide sequence that encodes the antibody or the antigen-binding fragment thereof described herein. Nonetheless, polynucleotides that vary due to differences in codon usage are expressly contemplated by the present disclosure. In certain embodiments, sequences that have been codon-optimized for mammalian expression are specifically contemplated. Codon optimization can be performed using known techniques and tools, e.g., the GenScript® OptimiumGene™ tool. Codon-optimized sequences include sequences that are partially codon-optimized (i.e., at least one codon is optimized for expression in the host cell) and those that are fully codon-optimized.

Therefore, in some embodiments, a mutagenesis approach, such as site-specific mutagenesis, may be employed to prepare variants and/or derivatives of the antibodies or antigen-binding fragments described herein. This approach allows specific modifications in a polypeptide sequence to be made through mutagenesis of the underlying polynucleotides that encode them. These techniques provide a straightforward approach to preparing and testing sequence variants, for example, incorporating one or more of the foregoing considerations by introducing one or more nucleotide sequence changes into the polynucleotide.

### Glycoengineering

Glycoforms of a therapeutic antibody have been shown to have a profound influence on the effector functions of the antibody, especially for those involved in antibody-dependent, cell-mediated cytotoxicity (ADCC). For example, N-glycan fucosylation at Asn 297 in the Fc domain is well known to affect ADCC activity. Evidence shows that a reduction in fucosylation can increase ADCC activity significantly via a strong binding between the Fc domain and an Fc gamma receptor IIIa (FcγIIIA). On the other hand, some studies suggest that terminal sialyation of the glycan on the antibody is critical to its effector functions, too, and the sialyation might have divergent functions in antibody classes. Accordingly, in some embodiments, the antibody of the present disclosure can be engineered to carry a desired glycoform.

Desired glycoforms can be obtained via in vitro glycoengineering or in vivo glycoengineering. In vitro glycoengineering can be enzymatic and chemoenzymatic. A typical enzymatic approach involves treating the produced antibodies with certain enzymes, for example, diphosphate galactose (UDP-Gal), β-1,4 galactosyltransferase-1 (B4GalT1), and α 2,6 sialyltransferase (ST6Gal1) to produce the G2S2F glycoform. In contrast, a chemoenzymatic approach for sialylated glycoforms comprises deglycosylation of N-linked glycans using native endoglycosidases followed by the addition of homogenous N-linked glycans using mutant endoglycosidases. In vivo glycoengineering usually requires cell culture medium systems and/or a gene-edited cell line. For example, to inhibit fucosylation, some suggest using a culture medium system comprising 2F-peracetyl-fucose. On the other hand, gene-edited cell lines with the endogenous FUT8 gene knocked out have been established to produce antibodies having glycoforms without the core-fucose. Examples regarding glycoengineering strategies can be found, for example, in PCT Patent Publication No. WO2009135181A2, David T. Ho et al., BioProcess International, 14 (4), April 2016, and Ravi Vattepu et al., Front. Immunol., 6 Apr. 2022, all are herein incorporated by reference in their entirety.

In some embodiments, the antibody of the present disclosure comprises a glycoform that does not have a core-fucose. In some embodiments, the antibody of the present disclosure comprises a sialylated glycoform. In certain embodiments, the antibody of the present disclosure comprises a α2-6 sialyl complex type (SCT) glycan.

### Multiple-Specificity and Fusion Protein

In certain embodiments, the antibody or the antigen-binding fragment thereof of the present disclosure is a multispecific antibody, such as a bispecific or trispecific antibody. Formats for bispecific antibodies are disclosed in, for example, Spiess et al., Mol. Immunol. 67 (2): 95 (2015), and in Brinkmann and Kontermann, mAbs 9 (2): 182-212 (2017), which bispecific formats and methods of making the same are herein incorporated by reference in their entirety and include, for example, Bispecific T cell Engagers (BiTEs), DARTs, Knobs-Into-Holes (KIH) assemblies, scFv-CH3-KIH assemblies, KIH Common Light-Chain antibodies, TandAbs, Triple Bodies, TriBi Minibodies, Fab-scFv, scFv-CH-CL-scFv, F(ab′)2-scFv2, tetravalent HCabs, Intrabodies, CrossMabs, Dual Action Fabs (DAFs) (two-in-one or four-in-one), DutaMabs, DT-IgG, Charge Pairs, Fab-arm Exchange, SEEDbodies, Triomabs, LUZ-Y assemblies, Fcabs, κλ-bodies, orthogonal Fabs, DVD-IgGs, IgG(H)-scFv, scFv-(H)IgG, IgG(L)-scFv, scFv-(L) IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, and DVI-IgG (four-in-one).

In some embodiments, the antibody of the present disclosure is a bi-specific, trispecific, or multiple-specific antibody, wherein at least one antigen-binding site of the bi-specific or multiple-specific antibody comprises the VH and VL of the present disclosure or the CDRs of the present disclosure. In such embodiments, another antigen-binding site of the bi-specific or multiple-specific antibody might target an antigen other than a Lewis Y antigen, such as an immune cell surface antigen, a tumor-associated antigen (which can be a protein, a glycan, or a glycoprotein), an immune checkpoint molecule, anti-angiogenesis molecule, or an immunomodulatory molecule. Examples of the tumor-associated antigen, the immune checkpoint molecule, and the immunomodulatory molecule include but are not limited to, programmed cell death 1 (PD-1), programmed death-ligand 1 (PD-L1), cytotoxic T-lymphocyte associated protein 4 (CTLA-4), epidermal growth factor receptor (EGFR), 4-1BB, CD3, CD28, CD40, HER2, B7H3, B7H4, B7H5, B7-H6, DLL3, Nectin-4, Trop-2, CEA, CA125, PSMA, VEGF, Globo H, SSEA-4, or SSEA-3.

Antibodies or antigen-binding fragments thereof of the present disclosure may, in certain embodiments, be comprised in a fusion protein that is capable of specific binding to a Lewis Y antigen as described herein. In some embodiments, a fusion protein is capable of expression at a surface of a host cell, e.g., a T cell, NK cell, or NK-T cell, and comprises an extracellular component comprising an antibody or antigen-binding fragment thereof as disclosed herein, and an intracellular component comprising an effector domain that is capable of directly or indirectly promoting an immunological response in a cell (e.g., immune system cell, such as a T cell) when receiving an appropriate signal (e.g., an effector domain from CD27, CD28, 4-1BB (CD137), OX40 (CD134), CD3ε, CD3δ, CD3ζ, CD25, CD27, CD28, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof), wherein the extracellular component and the intracellular component are connected by a transmembrane domain (e.g., a CD8 transmembrane domain, a CD4 transmembrane domain, a CD27 transmembrane domain, or a CD28 transmembrane domain) and the intracellular component optionally comprises a costimulatory domain or portion thereof selected from CD27, CD28, 4-1BB (CD137), OX40 (CD134), or a combination thereof. In certain embodiments, an extracellular component of a fusion protein comprising an antibody or antigen-binding fragment of the present disclosure comprises a polypeptide derived from an immunoglobulin protein; e.g., an IgG4 hinge-CH2-CH3. as stated above in an expression system in order to express a particular polypeptide such as the antibody or the antigen-binding fragment thereof or the pro-antibody as described herein.

In these and related embodiments, an antigen-binding domain may comprise a herein described antigen-binding fragment or antigen-binding moiety such as an scFv, and the extracellular component may further comprise a connector region comprising a hinge; e.g., in a chimeric antigen receptor molecule (CAR), which may be expressed on a cell surface of a host cell such as a T cell, a NK cell, or a NK-T cell for use in a cellular immunotherapy. CAR molecules and principles of design are described in, for example: Sadelain et al., Cancer Discov., 3 (4): 388 (2013); Harris and Kranz, Trends Pharmacol. Sci., 37 (3): 220 (2016); Stone et al., Cancer Immunol. Immunother., 63 (11): 1163 (2014); Xu et al., 2018 Oncotarget 9:13991; Androulla et al., 2018 Curr. Pharm. Biotechnol. Volume 19 (April 2018); Wu et al., 2016 Expert Opin. Biol. Ther. 16:1469; Ren et al., 2017 Protein Cell 8:634; which CAR molecules, CAR designs, and CAR design principles are herein incorporated by reference in their entirety. Some specific embodiments of scFV according to the present disclosure may comprise a construct having SEQ ID NO: 44 or SEQ ID NO: 45. The two sequences constitute a HKM4 VH-HKM4 VL scFV and a HKM1 VH-HKM1 VL scFV, respectively. While in other embodiments, a construct with a VL-VH orientation can also be made.

### Detection

In the present disclosure, any of the disclosed antibodies, antigen-binding fragments thereof, pro-antibody, and antibody conjugates may be linked to a detectable label (e.g., in addition to a detectable or therapeutic payload molecule of an antibody conjugate). In “direct detection”, only one detectable antibody is used, i.e., a primary detectable antibody. Thus, direct detection means that the antibody that is conjugated to a detectable label may be detected, per se, without the need for the addition of a second antibody (secondary antibody).

In contrast, indirect detection requires the application of one or more additional antibodies, i.e., secondary antibodies, after the application of the primary antibody. Thus, the detection is performed by the detection of the binding of the secondary antibody or binding agent to the primary detectable antibody. Examples of primary detectable binding agents or antibodies requiring the addition of a secondary binding agent or antibody include enzymatic detectable binding agents and hapten detectable binding agents or antibodies.

### Pro-Antibody

Systemic administration of mAb drugs may induce severe adverse events due to the high specificity and long half-life nature thereof. For treatments using an anti-Lewis Y antibody, the risk of gastrointestinal and hematological toxicities has been aware. Therefore, one aspect of the present disclosure is directed to a pro-antibody (or a lock antibody) aiming to improve the binding selectivity of anti-Lewis Y antibodies. A pro-antibody is an antibody or antigen binding fragment thereof that is inactivated and activable in a desired region of an organism where certain molecules or proteases exist (e.g., a proteolytic protease-overexpressed diseased tissue); therefore, a pro-antibody is also called an activable antibody. In some embodiments, the pro-antibody of the present disclosure comprises an antigen-binding moiety configured to bind a Lewis Y antigen and a masking moiety configured to inhibit the antigen-binding moiety from binding to the Lewis Y antigen, wherein the masking moiety is linked to the antigen-binding moiety via a linker.

### The Antigen-Binding Moiety

The antigen-binding moiety of the pro-antibody is capable of binding a Lewis Y antigen specifically. In some embodiments, the antigen-binding moiety is the antibody or the antigen-binding fragment thereof of the present disclosure, as described herein. In some other embodiments, the antigen-binding moiety of the pro-antibody comprises a heavy chain variable region comprising an amino acid sequence having at least 90%, 91%, 92%, 93%, 94%, 95%, 96%, 97%, 98%, or 99% percent identity to SEQ ID NO: 15 and a light chain variable region comprising an amino acid sequence having at least 90%, 91%, 92%, 93%, 94%, 95%, 96%, 97%, 98%, or 99% percent identity to SEQ ID NO: 11.

Yet in some other embodiments, the antigen-binding moiety of the pro-antibody comprises a heavy chain variable region comprising a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 17, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 18, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 19; and a light chain variable region (VL) comprising a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 22, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 23, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 24. In such embodiments, the antibody or the antigen-binding fragment thereof of the pro-antibody might comprises a heavy chain variable region comprising an amino acid sequence having at least 90%, 91%, 92%, 93%, 94%, 95%, 96%, 97%, 98%, or 99% percent identity to SEQ ID NO: 20 and a light chain variable region comprising an amino acid sequence having at least 90%, 91%, 92%, 93%, 94%, 95%, 96%, 97%, 98%, or 99% percent identity to SEQ ID NO: 25.

In some embodiments, the pro-antibody comprises a means for binding a Lewis Y antigen. In certain embodiments, the means for binding a Lewis Y antigen binds Lewis Y at a dissociation constant (K_{D}) of less than or equal to 10⁻³ M, 10⁻⁴ M, 10⁻⁵ M, 10⁻⁶ M, 10⁻⁷ M, or 10⁻⁸ M, or any ranges defined by the aforesaid two endpoints, such as 10⁻³ M to 10⁻⁸ M, 10⁻³ M to 10⁻⁷ M, 10⁻³ M to 10⁻⁶ M, 10⁻³ M to 10⁻⁵ M, 10⁻³ M to 10⁻⁴ M, 10⁻⁴ M to 10⁻⁸ M, 10⁻⁴ M to 10⁻⁷ M, 10⁻⁴ M to 10⁻⁶ M, 10⁻⁴ M to 10⁻⁵ M, 10⁻⁵ M to 10⁻⁸ M, 10⁻⁵ M to 10⁻⁷ M, 10⁻⁵ M to 10⁻⁶ M, 10⁻⁶ M to 10⁻⁸ M, or 10⁻⁶ M to 10⁻⁷M. In some embodiments, the K_{D} is about 10⁻⁶ M to 10⁻⁸ M. In certain embodiments, the means for binding a Lewis Y antigen does not bind Lewis X antigen.

### Masking Moiety

To improve the binding selectivity of an anti-Lewis Y antibody or the antigen-binding fragment thereof, a masking moiety (which is also often referred to as a “lock”) is configured to interfere, structurally or functionally, with the binding between the antibody or the antigen-binding fragment thereof and an antigen in normal tissues. According to its mechanism of interference, a masking moiety can be categorized into two groups.

A special hindrance-based masking moiety is designed to interfere with the binding by covering or blocking the antigen-binding sites of the antibody. Examples of a special hindrance-based masking moiety include but are not limited to human immunoglobulin G1 (IgG1) hinge, a leucine-rich and parallel heterodimeric coiled-coil domain, a non-antibody protein fragment (such as a latency-associated peptide (LAP) derived from transforming growth factor-β (TGF-β)), or an antibody fragment having a different antigen-binding affinity from the antibody to be blocked. Special hindrance-based masking moieties can be as those described in Lu Y C, et al., Specific activation of pro-infliximab enhances selectivity and safety of rheumatoid arthritis therapy. PLOS Biol. 2019; 17 (6): e3000286, Trang V H, et al., A coiled-coil masking domain for selective activation of therapeutic antibodies. Nat Biotechnol. 2019; 37 (7): 761-765, Chen I J, et al., Selective antibody activation through protease-activated pro-antibodies that mask binding sites with inhibitory domains. Sci Rep. 2017; 7 (1): 11587, and Metz S, et al., Bispecific antibody derivatives with restricted binding functionalities that are activated by proteolytic processing. Protein Eng Des Sel. 2012; 25 (10): 571-580, which are incorporated herein by reference in their entirety.

On the other hand, an affinity peptide-based masking moiety is a peptide designed to bind the antigen-binding site of the antibody. While the peptide binds the antigen-binding site of the antibody, it prevents the antibody from binding to antigens until the linker linking the peptide to the antibody is cleaved. However, given the peptide's affinity to the antigen-binding site of the antibody, the peptide might not be released from the antibody even after the linker is cleaved. Affinity peptide-based masking moieties can be as those described in Desnoyers L R, et al., Tumor-specific activation of an EGFR-targeting probody enhances therapeutic index. Sci Transl Med. 2013; 5 (207): 207ra144, Liu B, et al., In-depth characterization of a pro-antibody-drug conjugate by LC-MS. Mol Pharm. 2016; 13 (8): 2702-2710, and Yang Y, et al., Preclinical studies of a pro-antibody-drug conjugate designed to selectively target EGFR-overexpressing tumors with improved therapeutic efficacy. MAbs. 2016; 8 (2): 405-413, which are incorporated herein by reference in their entirety.

In some embodiments, the masking moiety comprises an amino acid sequence having at least 90%, 91%, 92%, 93%, 94%, 95%, 96%, 97%, 98%, or 99% percent identity to SEQ ID NO: 27. While in certain embodiments, the masking moiety is modified to reduce post-translation O-glycosylation. Without wishing to be bound by theories, O-glycosylation could lead to heterogeneity in the produced proteins or antibodies, making it difficult to manage from the perspective Chemistry, Manufacturing, and Controls (CMC). Therefore, in some embodiments, at least one of the Serine(S) or Threonine (T) of the masking moiety is replaced with an amino acid other than Serine(S) and Threonine (T).

In certain embodiments, the 4^{th}, the 8^{th}, and the 10^{th} amino acids of the masking moiety of SEQ ID NO: 27 are Serine and Threonine, and at least one of the 4^{th}, the 8^{th}, and the 10^{th} amino acids is replaced with an amino acid other than Serine and Threonine, for example, at least one of the 4^{th}, the 8^{th}, and the 10^{th} amino acids are replaced with Alanine (A) or Glycine (G). In certain embodiments, only one of the 4^{th}, the 8^{th}, and the 10^{th} amino acids is replaced with Alanine or Glycine, provided that the 8^{th} amino acid of the linker is not Alanine. In certain embodiments, all of the 4^{th}, the 8^{th}, and the 10^{th} amino acids are replaced with Alanine or Glycine. In certain embodiments, the masking moiety comprises an amino acid sequence having at least 90%, 91%, 92%, 93%, 94%, 95%, 96%, 97%, 98%, or 99% percent identity to the amino acid sequence set forth in SEQ ID NO: 28.

In some embodiments, the pro-antibody, without being activated binds Lewis Y at a dissociation constant (K_{D}) of less than or equal to 10⁻³ M, 10⁻⁴ M, 10⁻⁵ M, or 10⁻⁶ M, or any ranges defined by the aforesaid two endpoints, such as 10⁻³ M to 10⁻⁶ M, 10⁻³ M to 10⁻⁵ M, 10⁻³ M to 10⁻⁴ M, 10⁻⁴ M to 10⁻⁶ M, 10⁻⁴ M to 10⁻⁵ M, or 10⁻⁵ M to 10⁻⁶ M. After activation, in some embodiments, the pro-antibody binds Lewis Y at a dissociation constant (K_{D}) of less than or equal to 10⁻³ M, 10⁻⁴ M, 10⁻⁵ M, 10⁻⁶ M, 10⁻⁷M, or 10⁻⁸ M, or any ranges defined by the aforesaid two endpoints, such as 10⁻³ M to 10⁻⁸ M, 10⁻³ M to 10⁻⁷ M, 10⁻³ M to 10⁻⁶ M, 10⁻³ M to 10⁻⁵ M, 10⁻³ M to 10⁻⁴ M, 10⁻⁴ M to 10⁻⁸ M, 10⁻⁴ M to 10⁻⁷ M, 10⁻⁴ M to 10⁻⁶ M, 10⁻⁴ M to 10⁻⁵ M, 10⁻⁵ M to 10⁻⁸ M, 10⁻⁵ M to 10⁻⁷ M, 10⁻⁵ M to 10⁻⁶ M, 10⁻⁶ M to 10⁻⁸ M, or 10⁻⁶ M to 10⁻⁷ M. In some embodiments, the K_{D} is about 10⁻⁶ M to 10⁻⁸ M.

### Linker

A linker is designed to conjugate the masking moiety to the antibody or the antigen-binding fragment thereof. To release the masking moiety from the antibody or the antigen-binding fragment thereof, the linker can be a protease-sensitive linker, a pH-sensitive linker, or a glutathione-sensitive linker. Examples of cleavable linkers might be those that are cleavable by a certain molecule or protease that exists in the targeted tissues to de-occupy the masking moiety from the antibody or the antigen-binding fragment thereof.

In some embodiments, the linker comprises a proteinase substrate flanked by a first linkage moiety and a second linkage moiety, and at least one of the first linkage moiety and the second linkage moiety comprises an amino acid sequence of PLAQ (SEQ ID NO: 37). Without wishing to be bound by theories, the present disclosure contemplates that a linker contributes to the shielding (blocking efficiency) of the masking moiety, activation efficiency of the pro-antibody, or therapeutic efficacy of the activated pro-antibody as it affects the overall structure of the pro-antibody. A proper linker usually has 10 to 30 amino acids, including the sequence of a proteinase substrate, and the actual sequences thereof might vary depending on the antibody or the antigen-binding moiety conjugated to. This is because, without wishing to be bound by theories, the present disclosure contemplates that antibodies targeting different antigens have different configurations (e.g., in the heavy chain/light chain variable regions) to one another, so a proper linker needs to be developed accordingly. As taught herein, it is believed that a linker comprising a linkage moiety that has a PLAQ peptide provides a particularly desired shielding (blocking efficiency) of the masking moiety, activation efficiency of the pro-antibody, or therapeutic efficacy of the activated pro-antibody, when the pro-antibody is configured to bind Lewis Y antigen. This is particularly true when the antigen-binding moiety of the pro-antibody is as described in the present disclosure.

In some embodiments, at least one of the first linkage moiety and the second linkage moiety comprises an amino acid sequence of Gly-Gly-Ser (GGS), and optionally, the first linkage moiety and the second linkage moiety can both comprise a GGS peptide. In some specific embodiments, the first linkage moiety comprises an amino acid sequence of PLAQG (SEQ ID NO: 38), and the second linkage moiety comprises an amino acid sequence of GGGGS (SEQ ID NO: 39). For example, the first linkage moiety might comprise an amino acid sequence of GGSPLAQG (SEQ ID NO: 40).

In some embodiments, the cleavable linker comprises a substrate of a protease overexpressed in a diseased tissue. Such a protease can be a Matric-metalloproteinase 2 (MMP2), Matric-metalloproteinase 7 (MMP7), Matric-metalloproteinase 9 (MMP9), Matric-metalloproteinase 13 (MMP13), urokinase (Upa), matriptase, Factor Xa, legumain, tissue plasminogen activator (tPA), or ADAM metalloproteinases. In some embodiments, the linker comprises a MMP2 substrate. In certain embodiments, the linker comprises an amino acid sequence having at least 90%, 91%, 92%, 93%, 94%, 95%, 96%, 97%, 98%, or 99% percent identity to SEQ ID NO: 41, SEQ ID NO: 42, or SEQ ID NO: 43, SEQ ID NO: 29, SEQ ID NO: 30, or SEQ ID NO: 31.

In some embodiments, the first linkage moiety connects the masking moiety to the proteinase substrate, and the second linkage moiety connects the proteinase substrate to the antigen-binding moiety. In some embodiments, the pro-antibody comprises a first linker comprising a first proteinase substrate flanked by the first linkage moiety and the second linkage moiety, and the pro-antibody further comprises a second linker comprising a second proteinase substrate flanked by a third linkage moiety and a fourth linkage moiety. The third linkage moiety and the fourth linkage moiety can be defined as the definition of the first linkage moiety and the second linkage moiety described herein. In such embodiments, the first linker might covalently connect the masking moiety to a heavy chain of the antigen-binding moiety (for example, linking the masking moiety to the N-terminal of the heavy chain), and the second linker might covalently connect the masking moiety to a light chain of the antigen-binding moiety (for example, linking the masking moiety to the N-terminal of the light chain). Without wishing to be bound by theories, having the first linker and the second linker, which respectively link the masking moiety to the antigen-binding moiety, provides better stability to block or intervene in the binding of the antigen-binding moiety to the targeted antigen/epitope, thereby providing better inhibitory effect.

### Nucleic Acids, Recombinant Vector, and Host Cell

One aspect of the present disclosure is directed to an isolated nucleic acid encoding the isolated antibody or the antigen-binding fragment thereof or the isolated pro-antibody of the present disclosure. The present disclosure also provides a recombinant vector comprising the isolated nucleic acid. Yet in another aspect, the present disclosure provides a host cell comprising the recombinant vector. In some embodiments, the nucleic acids of the present disclosure may comprise DNA or RNA.

### Nucleic Acids

In one aspect, the isolated nucleic acid of the present disclosure is configured to encode the isolated antibody or the antigen-binding fragment thereof of the present disclosure or encode the isolated pro-antibody of the present disclosure. In some embodiments, the isolated nucleic acid can comprise a polynucleotide configured to encode the VH of the present disclosure. In yet some embodiments, the isolated nucleic acid comprises a polynucleotide encoding the heavy chain of the present disclosure's antibody. In some embodiments, the isolated nucleic acid comprises a polynucleotide encoding the VL of the present disclosure's antibody. In yet some embodiments, the isolated nucleic acid comprises a polynucleotide encoding the light chain of the present disclosure. In certain embodiments, the isolated nucleic acid comprises a polynucleotide encoding the heavy chain and the light chain of the present disclosure. In some embodiments, the isolated nucleic acid comprises a first polynucleotide encoding the heavy chain of the present disclosure and a second polynucleotide encoding the light chain of the present disclosure.

In some embodiments, the isolated nucleic acid or the fragment thereof is codon-optimized for expression in a host cell. In some embodiments, the isolated nucleic acid of the present disclosure comprises a polynucleotide variant, which has different nucleotide sequences but nonetheless encodes substantially the same polypeptide, which can be the VH, VL, heavy chain, or light chain of the present disclosure. One skilled in this art will recognize that alternation in the nucleotide sequences can be appropriately determined by taking into account codon degeneracy, amino acid similarity, reading frame positioning, and the like.

The isolated nucleic acid described herein, or fragments thereof, regardless of the length of the coding sequence itself, may be combined with other nucleic acid sequences, such as promoters, polyadenylation signals, restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative nucleic acid with total lengths of about 10,000, about 5000, about 3000, about 2,000, about 1,000, about 500, about 200, about 100, about 50 base pairs in length, and the like (including all intermediate lengths) are contemplated to be useful.

In certain embodiments, the polynucleotide encoding the VH of the present disclosure comprises a sequence having at least 70%, 75%, 80%, 85%, 90%, 91%, 92%, 93%, 94%, 95%, 96%, 97%, 98%, or 99% percent identity to SEQ ID NO: 6. In certain embodiments, the polynucleotide encoding the heavy chain of the present disclosure comprises a sequence having at least 70%, 75%, 80%, 85%, 90%, 91%, 92%, 93%, 94%, 95%, 96%, 97%, 98%, or 99% percent identity to SEQ ID NO: 7. In certain embodiments, the polynucleotide encoding the VL of the present disclosure comprises a sequence having at least 70%, 75%, 80%, 85%, 90%, 91%, 92%, 93%, 94%, 95%, 96%, 97%, 98%, or 99% percent identity to SEQ ID NO: 13. In certain embodiments, the polynucleotide encoding the light chain of the present disclosure comprises a sequence having at least 70%, 75%, 80%, 85%, 90%, 91%, 92%, 93%, 94%, 95%, 96%, 97%, 98%, or 99% percent identity to SEQ ID NO: 14.

In another example, the present disclosure provides an isolated nucleic acid configured to encode the pro-antibody of the present disclosure. In some embodiments, the isolated nucleic acid comprises a first polynucleotide configured to encode the antigen-binding moiety of the pro-antibody. In some embodiments, the isolated polynucleotide further comprises a second polynucleotide configured to encode the masking moiety. In yet some embodiments, the isolated nucleic acid further comprises a third polynucleotide configured to encode the linker.

### Recombinant Vector

The present disclosure provides a recombinant vector comprising the isolated nucleic acid of the present disclosure. In some embodiments, the recombinant vector comprises an isolated polynucleotide encoding a heavy chain variable region, VH CDRs, or a heavy chain as described herein. In some embodiments, the recombinant vector comprises an isolated polynucleotide encoding a light chain variable region, VL CDRs, or a light chain as described herein. In certain embodiments, the recombinant vector comprises a first polynucleotide encoding the heavy chain of the antibody or the antigen-binding fragment thereof of the present disclosure and a second polynucleotide encoding the light chain of the antibody or the antigen-binding fragment thereof of the present disclosure.

A recombinant vector, as described herein, is a vehicle configured to carry and/or express a nucleic acid-type cargo (e.g., a mRNA molecule) in a host cell or in vitro. The recombinant vector can be but is not limited to a nucleic acid, a plasmid, a virus, a lipid-based vehicle (such as a liposome, a noisome, a nanoemulsion, a solid lipid nanoparticle (SLN), a nanostructured lipid carrier (NLC), or a lipid nanoparticle (LNP)), a polymersome, or a bead (e.g., for cell-free expression). In certain embodiments, the recombinant vector is configured to be capable of transforming into a host cell where the nucleic acid cargo is expressed. In some specific embodiments, the recombinant vector is an expression vector, which, in addition to the nucleic acid encoding the antibody or the antigen-binding fragment thereof or the pro-antibody of the present disclosure, further comprises a control sequence as described herein or elements for expression, such as a promoter, a ribosomal binding site, a start codon, a terminal code, or a transcription terminal sequence. The expression vector can also comprise an origin of replication, a selectable marker, and/or a multiple cloning site. In some embodiments, the recombinant vector provides targeted delivery of the cargo to a desired region in a subject.

In some embodiments, the recombinant vector is a plasmid, which can be a eukaryotic plasmid or a prokaryotic plasmid. Examples of plasmids include, but are not limited to, a pET series plasmid, a pBR322 series plasmid, a pUC series plasmid, a pCDNA series plasmid, or a pCI series plasmid.

In some embodiments, the recombinant vector is a viral particle, which includes but is not limited to a retrovirus, adenovirus, parvovirus (e.g., adeno-associated viruses), coronavirus, negative-strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

### Host Cell

The present disclosure provides a host cell comprising the recombinant vector of the present disclosure. In some embodiments, the host cell is a bacterial cell (e.g., E. coli), a yeast cell, an insect cell, or a mammalian cell (e.g., a CHO cell, a HEK293 cell, or an immune cell, such as a T cell, an NK cell, or an NK-T cell). The present disclosure also provides, in certain embodiments, a method that comprises using a construct/recombinant vector.

The term “host cell” is used to refer to a cell into which has been introduced, or which is capable of having introduced into it, a nucleic acid sequence encoding one or more of the herein described antibodies and antigen-binding fragments thereof, and which further expresses or is capable of expressing a selected gene of interest, such as a gene encoding any herein described antibody or antigen-binding fragment. The term includes the progeny of the parent cell, whether or not the progeny is identical in morphology or in genetic make-up to the original parent, so long as the selected gene is present. Accordingly, there is also contemplated a method comprising introducing such nucleic acid into a host cell. The introduction may employ any available technique. For eukaryotic cells, suitable techniques may include calcium phosphate transfection, DEAE-Dextran, electroporation, liposome-mediated transfection, and transduction using retrovirus or another virus, e.g., vaccinia or, for insect cells, baculovirus. For bacterial cells, suitable techniques may include calcium chloride transformation, electroporation, and transfection using bacteriophage. The introduction may be followed by causing or allowing expression from the nucleic acid, e.g. by culturing host cells under conditions for expression of the gene. In one embodiment, nucleic acid is integrated into the genome (e.g. chromosome) of the host cell. Integration may be promoted by the inclusion of sequences that promote recombination with the genome, in accordance with standard techniques.

In some embodiments, the host cell comprises one recombinant vector of the present disclosure comprising a nucleic acid encoding the antibody or the antigen-binding fragment thereof or the pro-antibody of the present disclosure. In some embodiments, the host cell comprises a first recombinant vector comprising a polynucleotide encoding a heavy chain of the antibody or the antigen-binding fragment thereof of the present disclosure and a second recombinant vector comprising a polynucleotide encoding a light chain of the antibody or the antigen-binding fragment thereof of the present disclosure.

### Antibody Conjugates

One aspect of the present disclosure is directed to an antibody conjugate. The antibody conjugate of the present disclosure comprises a payload molecule covalently linked to an isolated antibody or an antigen-binding fragment thereof or a pro-antibody of the present disclosure. In some embodiments, the antibody or the antigen-binding fragment thereof or the pro-antibody comprises a native amino acid (e.g., a lysine conjugation or a cysteine conjugation), a non-native cysteine, a non-native non-natural amino acid, or a glycoform existing or configured for site-specific conjugation. Without wishing to be bound by theories, binding of the isolated antibody or the antigen-binding fragment thereof or the pro-antibody (e.g., after activation) to an antigen initiates internalization of the antigen conjugate into the cytosol or a lysosomal compartment of a target cell that expresses the antigen, thereby allowing targeted delivery of the payload molecule.

### Conjugation

Various techniques may be used to couple a payload molecule to an antibody or antigen-binding fragment thereof to form an antibody conjugate of the present disclosure. In some embodiments, an antibody conjugate comprises a payload molecule that is covalently linked by a linker to the antibody or antigen-binding fragment thereof. In some embodiments, the conjugation site of a payload on the antibody of the present disclosure is away from the antigen-binding site of the antibody, so the antigen-binding affinity will not be affected.

Lysine and Cysteine conjugation. In some embodiments, the payload is conjugated, for example, via a linker, to a lysine residue or a cysteine residue of the antibody. Lysine residue is commonly used for conjugation for its high natural abundance, surface accessibility, and the nucleophilicity of the e-amino side chain. For lysine conjugation, a linker having a N-Hydroxysuccinimide (NHS) ester moiety is often used. On the other hand, cysteine conjugation offers a particularly attractive target for protein bioconjugation due to their low natural abundance and the exceptionally high nucleophilicity of the deprotonated thiolate side chain. Cysteine conjugation is most commonly made via 1,4-conjugate addition to N-substituted maleimides, which are particularly attractive reagents due to their synthetic accessibility and rapid reaction rates with cysteine under mild conditions. In addition, genetically modifying an antibody surface with accessible cysteine residues on an antibody surface has emerged as a popular method to achieve the desired site-selective and homogeneous modification. More information regarding lysine and cysteine conjugation or other conjugation approaches is described in Walsh S J, et. al., Site-selective modification strategies in antibody-drug conjugates. Chem Soc Rev. 2021 Jan. 21; 50 (2): 1305-1353, which is herein incorporated by reference in its entirety.

Glycan conjugation. In some embodiments, the payload is conjugated, for example, via a linker, to a glycan on a surface of the antibody of the present disclosure. For example, the antibody might have a glycan attached to the N297 site thereof (i.e., located in the CH2 domain of the antibody). Without wishing to be bound by theories, conjugation via a glycan on the antibody surface can be beneficial because the conjugation is away from the antigen-binding site. Besides, the glycosylation pattern is usually well conserved across antibody types, which simplifies widespread modification of antibodies; and the carbohydrate is chemically distinct from the polypeptide backbone of the antibody, enabling site-specific conjugation. More information regarding glycan conjugation or other conjugation approaches is described in Walsh S J, et. al., Site-selective modification strategies in antibody-drug conjugates. Chem Soc Rev. 2021 Jan. 21; 50 (2): 1305-1353, which is herein incorporated by reference in its entirety.

Linker. Linkers used in antibody conjugates are typically organic compounds that fall into one of two groups, organized according to the mechanism by which the payload molecule is released from the carrier molecule: cleavable linkers or non-cleavable linkers. Cleavable linkers are designed to be selectively degraded or cleaved according to an inherent property of the target cell. Non-cleavable linkers rely on non-specific degradation of the antibody conjugate to release the payload molecule.

Three types of cleavable linkers are protease-sensitive linkers, pH-sensitive linkers, and glutathione-sensitive linkers. The cleavage of the protease-sensitive linkers depends on the proteases present in a tumor cell lysosome thereby releasing the payload molecule. Some examples of protease-sensitive linkers include, but not limited to a valine-citrulline or valine-alanine or phenylalanine-lysine dipeptide or a tetrapeptide (e.g., GGFG or GFLG or ALAL). On the other hand, pH-sensitive linkers contain an acid labile group that is selectively hydrolyzed by the lower pH of endosomal and lysosomal compartments, relative to cytosolic pH, resulting in releasing pH in a cell. Glutathione-sensitive linkers comprise a disulfide bridge that can be reduced by intracellular glutathione; hance releases the payload molecule. In some embodiments, a linker further comprises a self-demolishing group, also referred to as a self-immolative group or a self-immolative spacer, to assist in a selective cleavage reaction. In certain embodiments, the self-demolishing group is para-amino benzyl alcohol (PABC).

Exemplary linkers, linker chemistries, and related mechanisms and methods are disclosed in Nareshkumar et al., Pharm. Res. 32:3526-3540 (2015), which compositions, methods, and techniques are herein incorporated by reference in their entirety.

A linker may be connected or coupled to the antibody or antigen-binding fragment thereof using any appropriate technique or mechanism. In some embodiments, a linker comprises a maleimide group (optionally PEGylated) capable of reacting with a reduced disulfide bridge in a hinge region of the antibody or antigen-binding fragment thereof. Other sites on the carrier molecule (i.e., the antibody or antigen-binding fragment thereof) suitable for conjugation to a linker may be introduced or engineered using recombinant techniques, such as introducing cysteine residues or non-natural amino acids for site-specific conjugation. Methods for introducing such modifications include, for example, the method described in Examples 6.3-7 of PCT Publication No. WO 2012/032181. Click chemistries useful for generating antibody conjugates include those described in Meyer et al., Bioconjug. Chem. 27 (12): 2791-2807 (2016), and are herein incorporated by reference in their entirety.

### Payload Molecule

In any of the antibody conjugates described herein, the payload molecule may be selected from a therapeutic agent (e.g., a cytotoxic or anti-proliferative agent) and a detectable indicator. Therapeutic agents suitable for cancer therapy include those disclosed in Parslow et al., Biomedicines 4:14 (2016), the payloads and ADC design principles in that publication are herein incorporated by reference. In certain embodiments, the payload molecule is a therapeutic agent selected from a tubulin-targeting anti-mitotic agent, a peptide-based toxin, a pyrrolobenzodiazepine (PBD) dimer, an antibiotic (e.g., calicheamicin), a pyrimidine synthesis inhibitor (e.g., 5-fluorouracil), an antimetabolite (e.g., methotrexate), a DNA alkylating agent (e.g., calicheamicins, pyrrolobenzodiazepines, duocarmycins), and a topoisomerase inhibitor (e.g., doxorubicin or camptothecin and derivatives thereof such as amantin, exatecan, 7-Ethyl-10-hydroxycamptothecin (SN38), deruxtecan (DXd), or PNU-159682). In further embodiments, the payload molecule is selected from mayntansinoid, auristatin, a PBD dimer, monomethylauristatin E (MMAE), monomethylauristatin F (MMAF), calicheamicin, 5-fluorouracil, methotrexate, doxorubicin, calicheamicins (e.g., Mylotarg®), pyrrolobenzodiazepines, duocarmycins, camptothecin, amantin, exatecan, 7-Ethyl-10-hydroxycamptothecin (SN38), deruxtecan (DXd), PNU-159682, DM1 (e.g., Trastuzumab-MCC-DM1), DM4 (e.g., ELAHERER), and derivatives thereof.

In some embodiments, the therapeutic agent is a heterobifunctional molecule comprising a first ligand and a second ligand interconnected to each other via a linker (e.g., a degrader-antibody conjugate). The first ligand might target a protein of interest (POI) of a target cell (e.g., a cancer cell), and the second ligand might engage a ubiquitous ligase (e.g., E3 ubiquitous ligase, such as the von Hippel-Lindau protein (VHL) or cereblon CRBN). Without wishing to be bound by theories, the design enables simultaneous binding of the therapeutic agent to the POI and the ubiquitous ligase resulting in ubiquitylation, which then triggers proteasome degradation. In some other embodiments, the therapeutic agent is an immune cell-targeting molecule, such as an agonist for Toll-like receptors (TLRs) and a stimulator of interferon genes (STING). These mediators can initiate signaling cascades that lead to cytokine production, thereby orchestrating and/or amplifying an antitumor immune response. Given the risk of systemic administration, the risk can be expectedly reduced by conjugating those mediators with antibodies for targeting delivery and internalization. Examples of the immune cell-targeting molecule include, but not limited to, BDC-1001 (a HER2-targeted ISAC with a TLR7/8 agonist payload), TAC-001 (a CD22-targeted ISAC with a chemically modified CpG TLR9 agonist), and XMT-2056 (a HER2-targeted ISAC with a dimericamidobenzimidazole (diABZI) STING agonist payload). Further information can be found in Tsuchikama, K., Anami, Y., Ha, S. Y. Y. et al. Exploring the next generation of antibody-drug conjugates. Nat Rev Clin Oncol 21, 203-223 (2024), which is herein incorporated by reference in its entirety.

In other embodiments, the payload molecule is a detectable indicator. In such embodiments, the antibody conjugate of the present disclosure can be used to detect a disease, such as cancer, either in vivo, in vitro, or ex vivo. Detectable indicators suitable for use in antibody conjugates, as well as related labeling strategies and imaging techniques (e.g., PET, MRI, NIR), include those disclosed in Friese and Wu, Mol. Immunol. 67 (200): 142-152 (2015) and Moek et al., J. Nucl. Med. 58: 83S-90S (2017), which are herein incorporated by reference in their entirety. In certain embodiments, the detectable indicator is selected from a radionuclide, a dye, a radiometal, a fluorescent moiety, an MRI contrast agent, a microbubble, a carbon nanotube, a gold particle, fluorodeoxyglucose, an enzyme, a chromophore, and a radio-opaque marker. In specific embodiments, the detectable indicator is a radionuclide selected from ⁶⁸Ga, ⁶⁴Cu, ⁸⁶Y, ⁸⁹Zr, ¹²⁴I, ^{99m}Tc, ¹²³I, ¹¹¹In, ¹⁷⁷Lu, ¹³¹I, ⁷⁶Br, ⁷⁸Zr, ¹⁸F, and ¹²⁴T. In certain such embodiments, an antibody conjugate further comprises a radionuclide chelator selected from maleimide-labeled DOTA, N-hydroxysuccinimide-DOTA, and desferrioxamine (DFO).

### Manufacture

The antibody conjugate can be manufactured according to methods known in the art, for example, L. Nathan Tumey, Antibody-Drug Conjugates: Methods and Protocols (2000), which is herein incorporated by reference in its entirety. Specifically in some embodiments, the antibody-drug conjugate of the present disclosure may be prepared by first obtaining a purified monoclonal antibody from a suitable host expression system, such as CHO cells. The antibody is then subjected to controlled chemical modification, for example partial reduction of disulfide bonds or activation of lysine residues, to generate conjugation-ready sites. A linker-drug intermediate comprising a cytotoxic payload is synthesized separately and covalently attached to the antibody under defined reaction conditions, thereby yielding a conjugated product with a desired drug-to-antibody ratio. The resulting conjugate is purified to remove excess linker-drug and free payload, and formulated in a pharmaceutically acceptable buffer to provide a stable antibody-drug conjugate suitable for therapeutic use.

In some embodiments, the antibody conjugate of the present disclosure comprises a drug-to-antibody ratio (DAR) of 0, 1, 2, 3, 4, 5, 6, 7, 8, or more. In certain embodiments, the present disclosure provides an antibody conjugate population comprises a plurality of antibody conjugates, wherein each antibody conjugate of the plurality is as described herein, and the antibody conjugate population comprises a drug-to-antibody ratio (DAR) of about 0, 1, 2, 3, 4, 5, 6, 7, or 8, or a range defined by two foregoing endpoints, such as, 0 to 8, 1 to 8, 2 to 8, 3 to 8, 4 to 8, 5 to 8, 6 to 8, 7 to 8, 1 to 7, 2 to 7, 3 to 7, 1 to 6, 2 to 6, 3 to 6, 2 to 5, or 3 to 5.

### Composition

One aspect of the present disclosure is directed to a composition. In one example, the composition comprises an antibody or an antigen-binding fragment thereof of the present disclosure. In another example, the composition comprises a pro-antibody of the present disclosure. Yet in another example, the composition comprises an antibody conjugate of the present disclosure. In some embodiments, the antibody or the antigen-binding fragment thereof, the pro-antibody, or the antibody conjugate can be comprised at an effective amount.

In some embodiments, a concentration of the antibody or the antigen-binding fragment thereof, the pro-antibody, or the antibody conjugate of the present disclosure in the composition is about 0.5, 1, 5, 10, 15, 20, 30, 50, 75, 100, 125, 150, 200, 250, 300, 350, 400, 450, 500, 550, 600, 650, 700, 750, 800, 900, 1000 mg/ml, or a range defined by two foregoing endpoints, such as, 0.5 to 1000, 0.5 to 900, 0.5 to 800, 0.5 to 700, 0.5 to 600, 0.5 to 500, 0.5 to 400, 0.5 to 300, 0.5 to 200, 0.5 to 150, 0.5 to 100, 0.5 to 50, 0.5 to 30, 0.5 to 20, 0.5 to 5, 0.5 to 1, 5 to 1000, 5 to 900, 5 to 800, 5 to 700, 5 to 600, 5 to 500, 5 to 400, 5 to 300, 5 to 200, 5 to 150, 5 to 100, 5 to 50, 5 to 30, 5 to 20, 5 to 10, 10 to 1000, 10 to 900, 10 to 800, 10 to 700, 10 to 600, 10 to 500, 10 to 400, 10 to 300, 10 to 200, 10 to 150, 10 to 100, 10 to 50, 10 to 30, 10 to 20, 50 to 1000, 50 to 900, 50 to 800, 50 to 700, 50 to 600, 50 to 500, 50 to 400, 50 to 300, 50 to 200, 50 to 150, 50 to 100, 100 to 1000, 100 to 900, 100 to 800, 100 to 700, 100 to 600, 100 to 500, 100 to 400, 100 to 300, 100 to 200, or 100 to 150 mg/ml.

In some embodiments, the composition of the present disclosure is a pharmaceutical composition, wherein the antibody or the antigen-binding fragment thereof, the pro-antibody, or the antibody conjugate of the present disclosure is the active ingredient. In certain embodiments, the pharmaceutical composition is for treating cancer. In some embodiments, the pharmaceutical composition is used in combination with another therapeutic agent. In some embodiments, the cancer is cancer expressing a Lewis Y antigen (i.e., a Lewis Y-expressing cancer), including but not limited to bladder cancer, colon cancer, pancreas cancer, stomach cancer, lung cancer, esophagus cancer, cervix cancer, head and neck cancer, uterus cancer, breast cancer, or liver cancer.

In such embodiments that the composition is a pharmaceutical composition, the composition can further comprise a non-active ingredient. The non-active ingredient can comprise the rest of the volume of the composition other than the active ingredient. In some embodiments, the non-active ingredient is a pharmaceutically acceptable excipient, including but not limited to a binder, a diluent, a disintegrant, a filler, a glidant, a lubricant, a coloring agent, a preservative, a sweetener, a surfactant, a solvent, a coating agent, or a combination thereof. In addition, the composition of the present disclosure can further comprise other pharmaceutically active ingredients (including other immunosuppressive agents as described elsewhere herein).

The composition of the present disclosure can be prepared by combining an antibody, an antigen-binding fragment, a pro-antibody, or an antibody conjugate of the present disclosure with an appropriate carrier, and may be formulated into preparations in solid, semi-solid, liquid, or microparticle- (e.g., microdroplet) containing gaseous forms, such as tablets, capsules, powders, granules, ointments, solutions, suppositories, injections, inhalants, gels, microspheres, and aerosols.

In some embodiments, the composition of the present disclosure is a reagent used to detect a cancer cell in vivo, ex vivo, or in vitro. In some embodiments, the composition of the present disclosure is used for a targeted delivery of a payload to a target cell or a target tissue that expresses Lewis Y antigen, wherein the target cell or the target tissue can be in an organism or in a sample collected from an organism.

### Method of Use

### Methods of Treating Cancer

One aspect of the present disclosure is directed to methods of using the antibody or the antigen-binding fragment, the pro-antibody, or the antibody conjugate of the present disclosure. In one example, the present disclosure provides a method for treating cancer, which comprises administering the antibody or the antigen-binding fragment, the pro-antibody, or the antibody conjugate of the present disclosure to a subject in need. In some embodiments, the antibody or the antigen-binding fragment, the pro-antibody, or the antibody conjugate is formulated as a composition (e.g., a pharmaceutical composition as described herein) for the administration.

In some embodiments, administering is conducted via a route selected from a group consisting of intravenous, parenteral, intragastric, intrapleural, intrapulmonary, intrarectal, intradermal, intraperitoneal, intratumoral, subcutaneous, oral, topical, transdermal, intracisternal, intrathecal, intranasal, and intramuscular. In some embodiments, the administering is performed once. In some other embodiments, the administering is performed more than once. In such embodiments, an interval of any two administrations is at least 3 hours, 6 hours, 12 hours, 24 hours, 1 day, 3 days, 7 days, 14 days, 1 month, 2 months, 3 months, 6 months, 1 year, or 2 years.

In some embodiments, the using the antibody or the antigen-binding fragment, the pro-antibody, or the antibody conjugate of the present disclosure is administered at an effective amount. The effective amount can be determined based on several factors, including but not limited to the condition of the subjects (ages, gender, species, body weight, health status, etc.), the progress of the disease to be treated, the administration route, the administration frequency, the dosage and interval of the administration, and the nature of the disease. In certain embodiments, the effective amount is about 0.1, 0.5, 1, 5, 10, 15, 20, 30, 50, 75, 100, 125, 150, 200, 250, 300, 350, 400, 450, 500, 550, 600, 650, 700, 750, 800, 900, 1000, 1500, or 2000 mg per body weight, or a range defined by two foregoing endpoints, such as, 0.1 to 2000, 0.1 to 1500, 0.1 to 1000, 0.1 to 900, 0.1 to 800, 0.1 to 700, 0.1 to 600, 0.1 to 500, 0.1 to 400, 0.1 to 300, 0.1 to 200, 0.1 to 150, 0.1 to 100, 0.1 to 50, 0.1 to 30, 0.1 to 20, 0.1 to 5, 0.1 to 1, 0.1 to 0.5, 0.5 to 2000, 0.5 to 1500, 0.5 to 1000, 0.5 to 900, 0.5 to 800, 0.5 to 700, 0.5 to 600, 0.5 to 500, 0.5 to 400, 0.5 to 300, 0.5 to 200, 0.5 to 150, 0.5 to 100, 0.5 to 50, 0.5 to 30, 0.5 to 20, 0.5 to 5, 0.5 to 1, 5 to 2000, 5 to 1500, 5 to 1000, 5 to 900, 5 to 800, 5 to 700, 5 to 600, 5 to 500, 5 to 400, 5 to 300, 5 to 200, 5 to 150, 5 to 100, 5 to 50, 5 to 30, 5 to 20, 5 to 10, 10 to 2000, 10 to 1500, 10 to 1000, 10 to 900, 10 to 800, 10 to 700, 10 to 600, 10 to 500, 10 to 400, 10 to 300, 10 to 200, 10 to 150, 10 to 100, 10 to 50, 10 to 30, 10 to 20, 50 to 2000, 50 to 1500, 50 to 1000, 50 to 900, 50 to 800, 50 to 700, 50 to 600, 50 to 500, 50 to 400, 50 to 300, 50 to 200, 50 to 150, 50 to 100, 100 to 2000, 100 to 1500, 100 to 1000, 100 to 900, 100 to 800, 100 to 700, 100 to 600, 100 to 500, 100 to 400, 100 to 300, 100 to 200, or 100 to 150 mg per body weight.

In some embodiments, the cancer is cancer expressing a Lewis Y antigen (i.e., a Lewis Y-expressing cancer), including but not limited to bladder cancer, colon cancer, pancreas cancer, stomach cancer, lung cancer, esophagus cancer, cervix cancer, head and neck cancer, uterus cancer, breast cancer, or liver cancer.

### Methods of Detecting Cancer

In another example, the present disclosure provides a method of detecting cancer, which comprises administering the antibody conjugate of the present disclosure to a subject in need, wherein the payload molecule of the antibody conjugate is a detectable indicator. In some embodiments, the method of detecting cancer is performed in vitro, and in such embodiments, the method comprises contacting the antibody conjugate of the present disclosure with a sample collected from a subject in need of such detection. In some embodiments, the antibody or the antigen-binding fragment, the pro-antibody, or the antibody conjugate is formulated as a composition as described herein for the use of the methods. In some embodiments, the sample is a biological sample, including but not limited to blood, serum, urine, saliva, plasma, cell, tissue, semen, feces, bone marrow, or skin.

In some embodiments, administering is conducted via a route selected from a group consisting of intravenous, parenteral, intragastric, intrapleural, intrapulmonary, intrarectal, intradermal, intraperitoneal, intratumoral, subcutaneous, oral, topical, transdermal, intracisternal, intrathecal, intranasal, and intramuscular. In some embodiments, the administering is performed once. In some other embodiments, the administering is performed more than once. In such embodiments, an interval of any two administrations is at least 3 hours, 6 hours, 12 hours, 24 hours, 1 day, 3 days, 7 days, 14 days, 1 month, 2 months, 3 months, 6 months, 1 year, or 2 years.

In some embodiments, the using the antibody or the antigen-binding fragment, the pro-antibody, or the antibody conjugate of the present disclosure is administered at an effective amount. The effective amount can be determined based on several factors, including but not limited to the condition of the subjects (ages, gender, species, body weight, health status, etc.), the progress of the disease to be treated, the administration route, the administration frequency, the dosage and interval of the administration, and the nature of the disease. In certain embodiments, the effective amount is about 0.1, 0.5, 1, 5, 10, 15, 20, 30, 50, 75, 100, 125, 150, 200, 250, 300, 350, 400, 450, 500, 550, 600, 650, 700, 750, 800, 900, 1000 mg per body weight, or a range defined by two foregoing endpoints, such as, 0.1 to 1000, 0.1 to 900, 0.1 to 800, 0.1 to 700, 0.1 to 600, 0.1 to 500, 0.1 to 400, 0.1 to 300, 0.1 to 200, 0.1 to 150, 0.1 to 100, 0.1 to 50, 0.1 to 30, 0.1 to 20, 0.1 to 5, 0.1 to 1, 0.1 to 0.5, 0.5 to 1000, 0.5 to 900, 0.5 to 800, 0.5 to 700, 0.5 to 600, 0.5 to 500, 0.5 to 400, 0.5 to 300, 0.5 to 200, 0.5 to 150, 0.5 to 100, 0.5 to 50, 0.5 to 30, 0.5 to 20, 0.5 to 5, 0.5 to 1, 5 to 1000, 5 to 900, 5 to 800, 5 to 700, 5 to 600, 5 to 500, 5 to 400, 5 to 300, 5 to 200, 5 to 150, 5 to 100, 5 to 50, 5 to 30, 5 to 20, 5 to 10, 10 to 1000, 10 to 900, 10 to 800, 10 to 700, 10 to 600, 10 to 500, 10 to 400, 10 to 300, 10 to 200, 10 to 150, 10 to 100, 10 to 50, 10 to 30, 10 to 20, 50 to 1000, 50 to 900, 50 to 800, 50 to 700, 50 to 600, 50 to 500, 50 to 400, 50 to 300, 50 to 200, 50 to 150, 50 to 100, 100 to 1000, 100 to 900, 100 to 800, 100 to 700, 100 to 600, 100 to 500, 100 to 400, 100 to 300, 100 to 200, or 100 to 150 mg per body weight.

In some embodiments, the cancer is cancer expressing a Lewis Y antigen (i.e., a Lewis Y-expressing cancer), including but not limited to bladder cancer, colon cancer, pancreas cancer, stomach cancer, lung cancer, esophagus cancer, cervix cancer, head and neck cancer, uterus cancer, breast cancer, or liver cancer.

### Other Definitions and Additional Information

The basic antibody unit is a heterotetrameric glycoprotein composed of two identical light (L) chains and two identical heavy (H) chains. Each L chain is linked to an H chain by at least one (and typically one) covalent disulfide bond, while the two H chains are linked to each other by one or more disulfide bonds depending on the H chain isotype. Each H and L chain also has regularly spaced intrachain disulfide bridges. Each H chain has at the N-terminus a variable domain (VH) followed by three constant domains (CH) for each of the α and γ chains and four CH domains for μ and ε isotypes. Each L chain has at the N-terminus, a variable domain (VL) followed by a constant domain (CL) at its other end. The VL is aligned with the VH and the CL is aligned with the first constant domain of the heavy chain (CH1). Particular amino acid residues are believed to form an interface between the light chain and heavy chain variable domains. The pairing of a VH and VL together forms a single antigen-binding site.

The L chain from any vertebrate species can be assigned to one of two clearly distinct types, called kappa (κ) and lambda (λ), based on the amino acid sequences of their constant domains (CL). Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (α), delta (δ), epsilon (ε), gamma (γ), and mu (μ), respectively. The γ and α classes are further divided into subclasses on the basis of relatively minor differences in CH sequence and function, e.g., humans express the following subclasses: IgG1, IgG2, IgG3, IgG4, IgA1, and IgA2. It will be appreciated that mammals encoding multiple Ig isotypes will be able to undergo isotype class switching.

An IgM antibody consists of five of the basic heterotetramer units along with an additional polypeptide called J chain and, therefore, contains ten antigen binding sites, while secreted IgA antibodies can polymerize to form polyvalent assemblages comprising two to five of the basic four-chain units along with a J chain. In the case of IgG, the four-chain unit general has a molecular weight of about 150,000 daltons. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The variable (V) domain mediates antigen binding and defines the specificity of a particular antibody for its particular antigen. The gene sequence encoding the VH domain has multiple copies of variable (V), diversity (D), and joining (J) segments. The gene sequence encoding the VL domain contains multiple copies of V and J segments. The VH and VL regions undergo gene rearrangement (i.e., somatic recombination) to develop diverse antigen specificity in antibodies. The term “variable” refers to the fact that certain segments of the V domains differ extensively in sequence among antibodies.

However, the variability is not evenly distributed across the 110-amino acid span of the variable domains. Instead, the V regions consist of relatively invariant stretches called framework regions (FRs) of 15-30 amino acids separated by short regions of extreme variability called “hypervariable regions.” These hypervariable regions are the result of somatic hypermutation during the affinity maturation process, and they are typically each 9-18 amino acids long. However, they have been found to range from 4-28 amino acids in length depending upon the particular epitope. For example, CDR3 regions up to at least 22 or 23 amino acids in length have been described. See, e.g., Morea V, et al., J Mol Biol. 275 (2): 269-94 (1998) and Kabat, E. A., et al., Sequences of Proteins of Immunological Interest, Fifth Edition. NIH Publication No. 91-3242 (1991). Antibody amino acid positions (e.g., CDR sequences) may be determined according to known numbering schemes, such as the Kabat, Chothia, IMGT, and/or EU numbering schemes.

The variable domains of native heavy and light chains each comprise four framework regions (FRs), largely adopting a β-sheet configuration, connected by three hypervariable regions (also known as complementarity determining regions (CDR) and defined further below), which form loops connecting, and in some cases forming part of, the β-sheet structure. The hypervariable regions in each chain are held together in close proximity by the FRs and, in some cases with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding of an antibody to an antigen, but exhibit various effector functions, such as participation of the antibody in antibody dependent cellular cytotoxicity (ADCC) or other mechanisms that may involve interaction of a constant region domain with cell surface Fc receptors (FcR).

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody is purified: (1) to greater than 95% by weight of antibody as determined by a conventional method, such as SEC-HPLC or the Bradford method, and most preferably more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or, silver stain. Isolated antibody includes the antibody in situ within recombinant cells since at least one component of the antibody's natural environment will not be present. Ordinarily, however, isolated antibody will be prepared by at least one purification step.

An “intact” antibody is one that comprises an antigen-binding site as well as a CL and at least heavy chain constant domains, C_{H}1, C_{H}2, and C_{H}3. The constant domains may be native sequence constant domains (e.g., human native sequence constant domains) or amino acid sequence variants thereof. Preferably, the intact antibody has one or more effector functions.

The term “hypervariable region,” when used herein, refers to the amino acid residues of an antibody that are responsible for antigen binding. The hypervariable region generally comprises amino acid residues from a “complementarity determining region” or “CDR” (e.g., as may be determined according to Kabat numbering, around about residues 24-34 (L1), 50-56 (L2) and 89-97 (L3) in the VL, and around about 28-36 (H1), 50-65 (H2) and 95-102 (H3) in the VH; see Kabat et al., Sequences of Proteins of Immunological Interest, 5^{th} Ed. Public Health Service, National Institutes of Health, Bethesda, Md. (1991); and/or according to methodologies known in the art for identifying CDRs as defined by Kabat, such as those described by Martin, “Protein Sequence and Structure Analysis of Antibody Variable Domains”, In Antibody Engineering, R. Kontermann and S. Dubel, 2001, Springer-Verlag, Berlin, Germany, pages 422-438) and/or those residues from a “hypervariable loop” (e.g., residues 26-32 (L1), 50-52 (L2) and 91-96 (L3) in the VL, and 26-32 (H1), 53-55 (H2) and 96-101 (H3) in the VH; Chothia and Lesk, J. Mol. Biol./96:901-917 (1987)).

An “antibody fragment” or “antigen-binding fragment of an antibody” is a polypeptide comprising or consisting of a portion of an intact antibody, preferably the antigen binding or variable region of the intact antibody. Examples of antibody fragments include an F(ab′)2 fragment, an Fv fragment, a single-chain Fv (ScFv) antibody, a diabody, minibody, nanobody (VHH), and a linear antibody (see U.S. Pat. No. 5,641,870; Zapata et al., Protein Eng. 8(10): 1057-1062 [1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH) and the first constant domain of one heavy chain (C_{H}1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Both the Fab and F(ab′)2 are examples of “antigen-binding fragments.” Fab′ fragments differ from Fab fragments by having an additional few residues at the carboxy terminus of the C_{H}1 domain, including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

The “Fc” fragment (or “Fc region” or “Fc domain”) comprises the carboxy-terminal portions (i.e., the C_{H}2 and C_{H}3 domains of IgG) of both H chains held together by disulfides. The effector functions of antibodies are determined by sequences in the Fc region. The Fc domain is the portion of the antibody recognized by cell receptors, such as the FcR, and to which the complement-activating protein, C1q, binds. As discussed herein, modifications (e.g., amino acid substitutions) may be made to an Fc domain in order to modify (e.g., improve, reduce, or ablate) one or more functionality of an Fc-containing polypeptide (e.g., an antibody of the present disclosure).

“Fv” is the minimum antibody fragment that contains a complete antigen-recognition and antigen-binding site. This fragment consists of a dimer of one heavy- and one light-chain variable region domain in tight, non-covalent association. From the folding of these two domains emanate six hypervariable loops (three loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although typically at a lower affinity than the entire binding site.

“Single-chain Fv” also abbreviated as “sFv” or “scFv”, are antibody fragments that comprise the VH and VL antibody domains connected into a single polypeptide chain. Preferably, the sFv polypeptide further comprises a polypeptide linker between the VH and VL domains that enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993). Other antibody fragments and molecules comprising the same include, for example, linear antibodies, tandem scFv, scFv-Fc, tandem scFv-Fc, scFv dimer, scFv-zipper, diabody-Fc, diabody-CH3, scDiabodies, scDiabody-Fc, scDiabody-CH3, nanobodies, TandAbs, minibodies, miniantibodies, triabodies, tetrabodies, scFab, Fab-scFv, Fab-scFv-Fc, scFv-CH-CL-scFv, and F(ab′)2-scFv2, all of which are also contemplated herein. As used herein, an antibody is said to be “immunospecific,” “specific for” or to “specifically bind” an antigen if it reacts at a detectable level with the antigen, preferably with an affinity constant, Ka, of greater than or equal to about 10⁴ M⁻¹, or greater than or equal to about 10⁵ M⁻¹, greater than or equal to about 10⁶ M⁻¹, greater than or equal to about 10⁷ M⁻¹, or greater than or equal to 10⁸ M⁻¹. Affinity of an antibody for its cognate antigen is also commonly expressed as a dissociation constant K_{D}, and, in certain embodiments, an antibody or an antigen-binding fragment thereof specifically binds to a Lewis antigen of the present disclosure if it binds with a K_{D} of less than or equal to 10⁻⁴ M, less than or equal to about 10⁻⁵ M, less than or equal to about 10⁻⁶ M, less than or equal to 10⁻⁷ M, or less than or equal to 10⁻⁸ M. Affinities of antibodies and antigen-binding fragments can be readily determined using conventional techniques, for example, those described by Scatchard et al. (Ann. N.Y. Acad. Sci. USA 51:660 (1949)), or by surface plasmon resonance (SPR) (e.g., Hearty et al., 2012 Meths. Mol. Biol. 907:411), by isothermal titration calorimetry (ITC) (e.g., Dam et al., 2008 J. Biol. Chem. 283:31366), by enzyme-linked immunosorbent assay (ELISA) (e.g., Bobrovnik, 2003 J. Biochem. Biophys. Meths. 75 (3): 213), or by other methodologies familiar to those skilled in the art.

Binding properties of an antibody to antigens, cells, or tissues thereof may generally be determined and assessed using immunodetection methods, including, for example, immunofluorescence-based assays, such as immunohistochemistry (IHC) and/or fluorescence-activated cell sorting (FACS). Other methods of determining the binding of an antibody to an antigen include, for example, enzyme-linked immunosorbent assay (ELISA), isothermal titration calorimetry (ITC), and surface plasmon resonance (SPR) techniques.

As used herein, “effector functions” refer to those biological activities attributable to the Fc region (a native sequence Fc region or amino acid sequence variant Fc region) of an antibody and varying with the antibody isotype. Examples of antibody effector functions include C1q binding and complement-dependent cytotoxicity, Fc receptor binding, antibody-dependent cell-mediated cytotoxicity (ADCC), phagocytosis; down-regulation of cell surface receptors (e.g., B cell receptor), B cell activation, and/or transglutaminase (Tgase)-mediated conjugation. Amino acid modifications (e.g., substitutions) to modify (e.g., improve, reduce, or ablate) Fc functionalities include, for example, the T250Q/M428L, M252Y/S254T/T256E, H433K/N434F, M428L/N434S, E233P/L234V/L235A/G236+A327G/A330S/P331S, E333A, S239D/A330L/1332E, P2571/Q311, K326W/E333S, S239D/1332E/G236A, N297A, N297S, N297D, N297Q, K322A, S228P, L235E+E318A/K320A/K322A, L234A/L235A, and L234A/L235A/P329G mutations, which mutations are summarized and annotated in “Engineered Fc Regions”, published by InvivoGen (2011) and available online at www.invivogen.com/PDF/review/review-Engineered-Fc-Regions-invivogen.pdf?utm_source=review&utm_medium=pdf&utm_campaign=review&utm_content=E ngineered-Fc-Regions, and are incorporated herein by reference.

The term “polynucleotide” or “nucleic acid” as referred to herein means a single-stranded or double-stranded nucleic acid polymer, and specifically includes single and double-stranded form of DNA. Polynucleotides can be generated, for example, by the polymerase chain reaction (PCR) or by in vitro translation, and fragments generated by any of ligation, scission, endonuclease action, or exonuclease action. In certain embodiments, the polynucleotides of the present disclosure are produced by PCR. Polynucleotides may be composed of monomers that are naturally occurring nucleotides (such as deoxyribonucleotides and ribonucleotides), analogs of naturally occurring nucleotides (e.g., α-enantiomeric forms of naturally-occurring nucleotides), or a combination of both. In further embodiments, nucleotides comprising a polynucleotide can be ribonucleotides or deoxyribonucleotides or a modified form of either type of nucleotide.

The term “isolated nucleic acid” as used herein shall mean a polynucleotide of genomic, cDNA, or synthetic origin or some combination thereof, wherein by virtue of its origin the isolated nucleic acid (1) is not associated with all or a portion of a polynucleotide in which the isolated nucleic acid is found in nature, (2) is linked to a polynucleotide to which it is not linked in nature, or (3) does not occur in nature as part of a larger sequence. Reference to a nucleotide sequence as set out herein encompasses a DNA molecule with the specified sequence, and an RNA molecule with the specified sequence in which U is substituted for T, unless context requires otherwise.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein-coding sequence is achieved under conditions compatible with the transcriptional activity of the control sequences.

The term “control sequence,” as used herein, refers to polynucleotide sequences that can affect the expression, processing, or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences, and polyadenylation sequences. In certain embodiments, “control sequences” can include leader sequences and/or fusion partner sequences. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

An “effective amount” of a composition refers to an amount sufficient, at dosages and for periods of time needed, to achieve the desired clinical results or beneficial treatment, as described herein. An effective amount may be delivered in one or more administrations. If the administration is to a subject already known or confirmed to have a disease or disease-state, the term “therapeutically effective amount” may be used in reference to treatment, whereas “prophylactically effective amount” may be used to describe administering an effective amount to a subject that is susceptible to or at risk of developing a disease or disease-state (e.g., recurrence) as a beneficial and/or protective course of reducing (e.g., in a statistically significant manner relative to an untreated state) the likelihood of occurrence and/or severity of the disease or disease-state.

As used herein, the terms “treat” and “treatment” refer to the medical management of a disease, disorder, or condition in a subject (human or non-human) (see, e.g., Stedman's Medical Dictionary). “Treatment” can also include prolonging survival relative to the expected survival of an untreated subject. Subjects in need of the disclosed methods and compositions include those diagnosed with the disease or disorder, those at risk of developing it, and those in need of prophylaxis to reduce the likelihood of occurrence or recurrence. Such treatment may reduce severity, partially or fully prevent disease onset, or decrease recurrence, without requiring absolute prevention. Clinical benefit of the disclosed compositions and methods may be assessed through in vitro assays, preclinical studies, and clinical trials in subjects for whom administration is intended, as described in the examples.

Treatment generally involves administering an antibody, antigen-binding fragment, pro-antibody, or antibody conjugate in an amount sufficient to provide therapeutic or prophylactic benefit. The appropriate dosage may vary across patient populations and requires further investigation to determine optimal regimens. For instance, a patient population with a cancer subtype expressing different levels of a target antigen may require adjusted dosing for effective treatment. Therapeutic or prophylactic benefit refers to any improved clinical outcome resulting from treatment or prevention, including prevention, delay, or reduction (e.g., a statistically significant decrease relative to an untreated control) of an undesired physiological change, disease, or disorder. Beneficial results include abatement, reduction, or alleviation of symptoms; decreased symptom occurrence; improved quality of life; extended disease-free status; reduction in disease extent; disease stabilization (i.e., lack of progression); delayed or slowed progression; amelioration or palliation of disease; partial or complete remission, whether detectable or undetectable; and improved overall survival.

A “detectable label” is a molecule or material that can produce a detectable (such as visually, electronically, or otherwise) signal that indicates the presence and/or concentration of the label in a sample. When conjugated to a peptide, the detectable label can be used to locate and/or quantify the target to which the specific peptide is bound. Thereby, the presence and/or concentration of the target in a sample can be detected by detecting the signal produced by the detectable label. A detectable label can be detected directly or indirectly, and several different detectable labels conjugated with different specific antibodies can be used in combination to detect one or more targets. Examples of detectable labels, which may be detected directly, include fluorescent labels, enzyme labels, radioisotopes, chemiluminescent labels, electrochemiluminescent labels, bioluminescent labels, polymers, polymer particles, metal particles, haptens, and dyes.

### EXAMPLES

### Example 1: Exemplary Antibody Generation and Characterization

The Example depicts the process conducted to identify and generate some exemplary anti-Lewis Y antibodies of the present disclosure. Babl/c mice were immunized with NCI-N87 cell lysate and Glycosphingolipids (GSL). The mice were then sacrificed, and their spleens were collected to isolate the splenocytes. The isolated splenocytes were fused with FO myeloma to obtain hybridoma clones. The hybridoma clones were then screened independently by using NCI-N87 GSL and AGS GSL (data not shown). Briefly, a 96-well plate was coated with 0.2 μg/well AGS GSL or NCI-N87 GSL in 95% EtOH and air-dry at room temperature overnight. After washing with 0.05% PBST, the plate was loaded with hybridoma supernatant and incubated at 37° C. for 1 hour. Following another round of washing, 3000× diluted Anti-Mouse IgG-Peroxidase Produced in Goat (Sigma) was added and incubated at 37° C. for 1 hour. After a final wash, TMB substrate was added and incubated at 37° C. for 10 minutes. The reaction was stopped with HCl, and the optical density (OD) was measured at 450-650 nm.

Three candidate hybridoma clones (FO1, FO10, and FO12) were identified with the desired binding profile and affinity (data not shown). The heavy chain and light chain sequences of the three clones were obtained, and recombinant antibodies having various combinations of heavy chain/light chain pairs of the three clones were made to select the best candidate antibody. The selected antibody (Ab12H1L) has the heavy chain from the FO12 clone and the light chain from the FO1 clone and was then humanized into two humanized IgG1 antibodies: HKM1 and HKM4, which have the same CDRs but differ from each other on the 42^{nd} and 44^{th} amino acids of the heavy chain variable region (See table below).

TABLE
Sequences of HKM1 and HKM4

| | |
|---|---|
| HKM4 VHCDR1 | DYYIY |
| (SEQ ID NO: 1) | |
| HKM4 VHCDR2 | YISQDGDITD YQDTLKG |
| (SEQ ID NO: 2) | |
| HKM4 VHCDR3 | GLSEGAWFAY |
| (SEQ ID NO: 3) | |
| HKM4 VH | EVQLVESGGG LVQPGGSLRL SCATSGFTES |
| (SEQ ID NO: 4) | DYYIYWVRQA PEKRLEWVAY ISQDGDITDY |
| | QDTLKGRFTI SRDNAKNTLY LQMNSLRAED |
| | TAVYYCARGL SEGAWFAYWG QGTLVTVSS |
| HKM4 Heavy | EVQLVESGGG LVQPGGSLRL SCATSGFTFS |
| chain | DYYIYWVRQA PEKRLEWVAY ISQDGDITDY |
| (SEQ ID NO: 5) | QDTLKGRFTI SRDNAKNTLY LQMNSLRAED |
| | TAVYYCARGL SEGAWFAYWG QGTLVTVSSA |
| | STKGPSVFPL APSSKSTSGG TAALGCLVKD |
| | YFPEPVTVSW NSGALTSGVH TFPAVLQSSG |
| | LYSLSSVVTV PSSSLGTQTY ICNVNHKPSN |
| | TKVDKKVEPK SCDKTHTCPP CPAPELLGGP |
| | SVELFPPKPK DTLMISRTPE VTCVVVDVSH |
| | EDPEVKENWY VDGVEVHNAK TKPREEQYNS |
| | TYRVVSVLTV LHQDWLNGKE YKCKVSNKAL |
| | PAPIEKTISK AKGQPREPQV YTLPPSRDEL |
| | TKNQVSLTCL VKGFYPSDIA VEWESNGQPE |
| | NNYKTTPPVL DSDGSFFLYS KLTVDKSRWQ |
| | QGNVFSCSVM HEALHNHYTQ KSLSLSPGK |
| HKM4 VLCDR1 | RSSQNIVHTN GNTYLE |
| (SEQ ID NO: 8) | |
| HKM4 VLCDR2 | KVSNRES |
| (SEQ ID NO: 9) | |
| HKM4 VLCDR3 | FQGSHVPFT |
| (SEQ ID NO: 10) | |
| HKM4 VL | DIVMTQTPLS LPVTLGQPAS ISCRSSQNIV |
| (SEQ ID NO: 11) | HTNGNTYLEW YLQKPGQSPQ LLIYKVSNRF |
| | SGVPDRESGS GSGTDFTLKI SRVEAEDVGV |
| | YYCFQGSHVP FTFGPGTKVD IK |
| HKM4 Light | DIVMTQTPLS LPVTLGQPAS ISCRSSQNIV |
| chain | HTNGNTYLEW YLQKPGQSPQ LLIYKVSNRF |
| (SEQ ID NO: 12) | SGVPDRFSGS GSGTDFTLKI SRVEAEDVGV |
| | YYCFQGSHVP FTFGPGTKVD IKRTVAAPSV |
| | FIFPPSDEQL KSGTASVVCL LNNFYPREAK |
| | VQWKVDNALQ SGNSQESVTE QDSKDSTYSL |
| | SSTLILSKAD YEKHKVYACE VTHQGLSSPV |
| | TKSFNRGEC |
| HKM1 VH | EVQLVESGGG LVQPGGSLRL SCATSGFTES |
| (SEQ ID NO: 15) | DYYIYWVRQA PGKGLEWVAY ISQDGDITDY |
| | QDTLKGRFTI SRDNAKNTLY LQMNSLRAED |
| | TAVYYCARGL SEGAWFAYWG QGTLVTVSS |

Generation of the antibody. The synthesized antibody sequences were constructed into pGNX expression plasmid using a restriction enzyme or HiFi assembly method. The plasmid DNAs were amplified with 400 mL culture in LB broth supplemented with 50 μg/mL Ampicillin and purified using NucleoBond Xtra Midi Plus kit (Macherey-Nagel). Antibodies were expressed via transient expression of HEK293 or CHO-DG44 cell expression system and purified by Amsphere™ A3 ProA resin (JSR), size-exclusion chromatography (HiLoad® 16/600 Superdex 200 pg Geometric column) and a post-protein A method. Sequences of antibodies are provided in the sequence list.

Glycan Antigen binding ELISA. To determine the specificity of the exemplary antibodies of the present disclosure, we tested its binding ability to several Lewis series glycans, including Lewis Y(LeY) tetraose, LeY-Gal pentaose, Lewis B (LeB)-Gal pentaose, Lewis X (LeX), Lewis A (LeA), and H type2. A 96-well plate was coated with 100 ng/ml of LeY-Gal-sp1-biotin (pentaose), LeY-PAA-biotin (tetraose), LeB-Gal-sp1-biotin (pentaose), LeX-PAA-biotin, LeA-PAA-biotin, or H type 2-PAA-biotin in PBS and incubated at 4° C. overnight. After washing with 0.05% PBST, the plate was loaded with antibody dilutions (15-0.05 μg/mL) and incubated at 37° C. for 1 hour. Following another round of washing, 10000× diluted Mouse anti-human IgG Fc-HRP (Southernbiotech) was added and incubated at 37° C. for 1 hour. After a final wash, TMB substrate was added and incubated at 37° C. for 10 minutes. The reaction was stopped with HCl, and the optical density (OD) was measured at 450-650 nm.

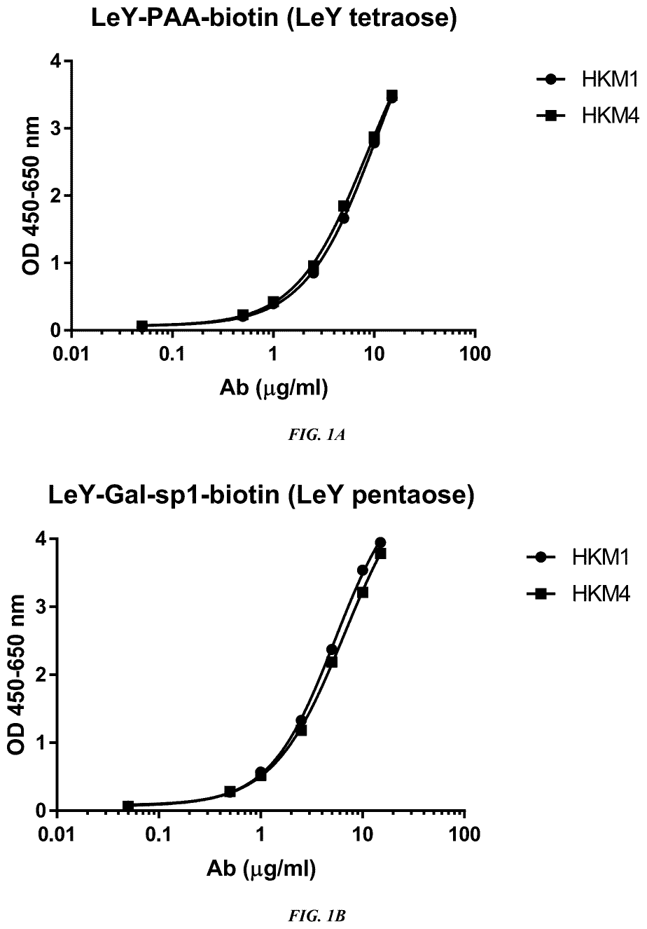

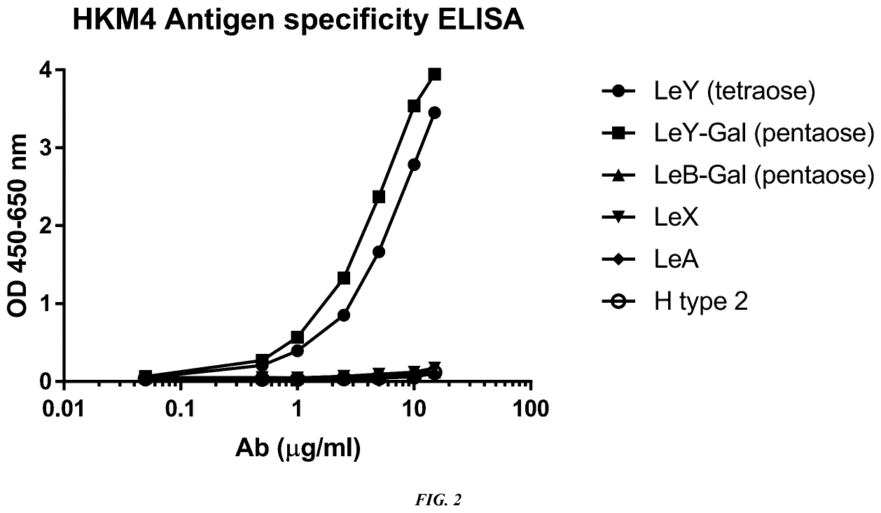

The results (FIG. 1A and FIG. 1B) show that both HKM1 and HKM4 bound Lewis Y-Gal pentaose and Lewis Y-tetraose, and the two exemplary antibodies exhibited similar binding affinity to the tested Lewis Y antigens. In addition, it was noted that HKM4 does not bind Lewis B-Gal (LeB) antigen, Lewis (LeX) X antigen, Lewis A (LeA) antigen, and H type 2 antigen (FIG. 2) even at a concentration higher than 10 μg/ml. These results confirm both HKM1 and HKM4 have the desired binding specificity.

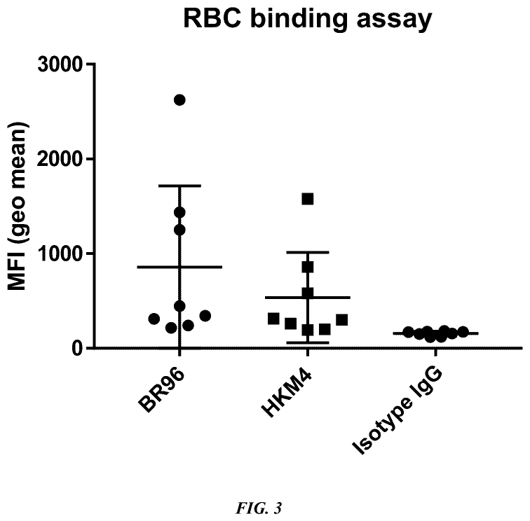

Red blood cell binding. Next, because Lewis Y is also expressed on red blood cells, the binding affinity of HKM4 to red blood cells was evaluated. The tested antibodies, including HKM4, BR96 (a conventional anti-Lewis Y antibody), and Isotype IgG (negative control; manufactured in-house), were prepared at a final 100 μg/ml concentration before use. Blood cells were collected using EDTA Blood Collection Tubes and diluted 100-fold in PBS. Then, the blood cells were mixed with the tested antibodies into a 96-well conical-bottom plate. The plate was incubated at room temperature for 30 minutes while protected from light. After incubation, the plate was centrifuged, the supernatant was discarded, and the cells were washed with PBS. This wash step was repeated, and the cells were then incubated with a 200-fold diluted Goat Anti-Human IgG Fcγ-FITC (Jackson ImmunoResearch) solution. Following another centrifugation and wash step, the cells were resuspended in PBS. The stained cells were analyzed using a Beckman CytoFLEX S flow cytometer. The results (FIG. 3) show that both the HKM4 antibody and the BR96 exhibited certain levels of binding affinity to red blood cells. Nevertheless, HKM4 exhibits significantly lower binding affinity to red blood cells at a Mean Fluorescence Intensity (MFI) of 535.88±478.31. In contrast, BR96 had an MFI of 858.69±856.40, and the negative control Isotype IgG (manufactured in-house) showed an MFI of 156.50±24.94. That is to say, on top of the superior binding affinity and specificity to Lewis Y antigens, HKM4 is also superior in having a lower risk of hematological toxicity. HKM4's binding affinity to red blood cells was only around 60% of that of BR96.

| Antibody | HKM4 | BR96 | Isotype IgG |
|---|---|---|---|
| MFI | 535.88 | 858.69 | 156.5 |
| Relative Binding to isotype control (%) | 342% | 549% | 100% |
| Relative Binding to BR96 (%) | 62% | 100% | |

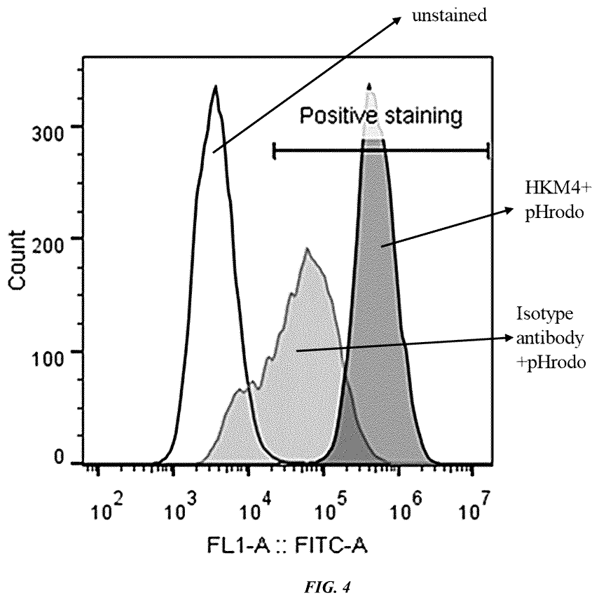

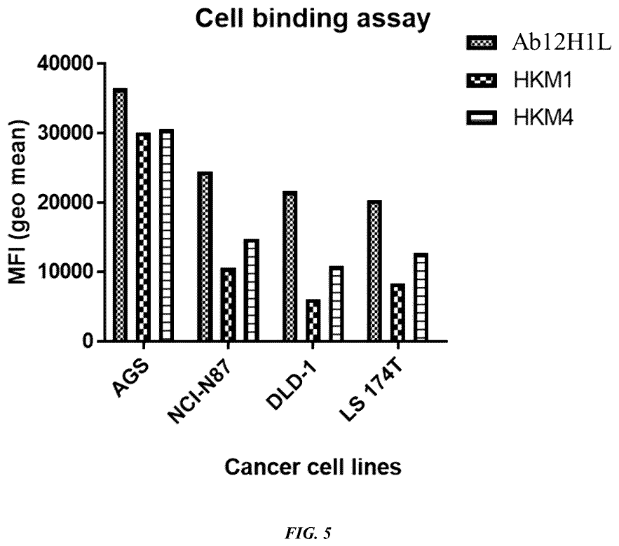

Internalization. In this experiment, the internalization of HKM4 upon binding to target cells was evaluated. Cells were seeded at a density of 1.5×10\hat{ }5 cells (AGS cell) per well and incubated in an incubator overnight (37° C., 5% CO₂). HKM4 antibodies were mixed with Zenon™ pHrodo™ iFL IgG Labeling Reagent (Invitrogen) and incubated for 5 minutes at room temperature. Then, the antibodies mixture was added to the cell culture. After coculturing the HKM4-pHrodo mixture with the cells at 37° C. for 24 hours, the cells were collected and suspended to a V-shaped bottom 96-well plate for analysis using Beckman CytoFLEX. The result (FIG. 4) shows that, compared to the isotype antibody (manufactured in-house) used as a control, HKM4 has a 99.99% peak shift compared to the unstained group. This data verifies that the binding of HKM4 to a target cell was able to trigger strong internalization, making HKM4 a great antibody for antibody-drug conjugates.

### Example 2: Anti-Tumor Activity

The experiments conducted in this example tested the binding of the antibodies of the present disclosure to cancer cells and its anti-tumor activity. The selected antibody Ab12H1L in Example 1 and its humanized antibodies, HKM1 and HKM4, were evaluated.

Cancer cell binding. A variety of cancer cell lines were tested in this experiment, including AGS (human gastric adenocarcinoma), NCI-N87 (human gastric adenocarcinoma), DLD-1 (colorectal adenocarcinoma), and LS 174T (colorectal adenocarcinoma). All cell lines were cultured according to the manuals before use. Cells were harvested from the culture and washed with phosphate-buffered saline (PBS). After being washed, the cells were resuspended in PBS containing 2% fetal bovine serum (FBS) to achieve a 2×10⁶ cells/mL density. Cells were then dispensed into each well of a plate and added with purified antibody samples. The plate was then incubated at 4° C. for 1 hour. After that, the wells of the plate were washed, and a secondary antibody (Goat Anti-Human IgG Fcγ-FITC) was added to the cells. The plate was then incubated at 4° C. for 30 minutes followed by washing and resuspending the cells in PBS. After that, the plate was analyzed using the Beckman cytoFLEX Software. The results (FIG. 5) verify that the antibodies of the present disclosure were capable of binding to a variety of cancer cells and especially showed strong binding to AGS cells. Ab12H1L exhibited binding affinities higher than HKM1 and HKM4, and HKM4 performed slightly better than HKM1. It is not uncommon that the humanization process would affect the binding affinity of the antibody. Since both HKM1 and HKM4 maintained the binding affinity, and the reduction in binding was only slightly, it was verified that the humanization process did not demolish the binding affinity.

Immunohistochemistry. The binding affinity of the antibodies of the present disclosure to different types of cancer cells was also evaluated using immunohistochemistry. In this experiment, a high-density multiple organs tumor with normal tissue array (Biomax, MC5004) having multiple tumor types and a Multiple organ normal tissue microarray (Biomax, FDA999x) were used. Tissue sections were deparaffinized by heating at 70° C. for 1 hour and immersed thrice in xylene. Subsequently, sections were washed in a buffer on a shaker for 5 minutes. Antigen retrieval was achieved by immersing the sections in a buffer and heating between 114° C. to 121° C. for 20 minutes using a pressure cooker, then cooling at room temperature. Afterward, sections were washed with buffer thrice for 5 minutes each. Sections were exposed to 3% hydrogen peroxide solution at room temperature for 20 minutes, followed by three washes with buffer. A diluted primary antibody was applied to each section and incubated at room temperature for 1 hour. After washing with buffer thrice, a secondary antibody (Mouse Anti-Human IgG Fc-HRP) was added and incubated for 1 hour at room temperature, followed by three washes. Sections were stained with DAB for 1 minute and counterstained with hematoxylin for 10 seconds. After washing, sections were air-dried and mounted with coverslips. The results showed (see table below), despite some binding affinities to normal tissues, HKM4 epitope was expressed in 37% of bladder (7/19), 35% of colon (7/20), 29% of pancreas (5/17), 28% of stomach (5/18), 22% of lung (4/18), 22% of esophagus (4/18), and 21% of cervix (4/19) tumor samples.

TABLE
HKM4 IHC staining

Tissue type

| | Tumor Positive % | Normal Positive % |
|---|---|---|
| Organ | (Score 1-3) | (Score 1-3) |
| Bladder | 7/19 = 37% | 4/5 = 80% |
| Colon | 7/20 = 35% | 0/5 = 0% |
| Pancreas | 5/17 = 29% | 2/5 = 40% |
| Stomach | 5/18 = 28% | 4/4 = 100% |
| Lung | 4/18 = 22% | 0/5 = 0% |
| Esophagus | 4/18 = 22% | 4/4 = 100% |
| Cervix | 4/19 = 21% | 1/1 = 100% |
| Head and Neck | 3/19 = 16% | 0/4 = 0% |
| Uterus | 2/20 = 10% | 0/3 = 0% |
| Breast | 1/19 = 5% | 1/1 = 100% |
| Liver | 1/18 = 6% | 0/5 = 0% |

Anti-tumor activity. In this experiment, HKM1 and HKM4 were evaluated for their anti-tumor activities using the AGS xenograft tumor inhibition model. Briefly, 5×10⁶ AGS cells mixed with 50% Matrigel were injected subcutaneously into female CB17 SCID mice for AGS tumor inhibition study (Day 0). After the average tumor weight reached 100 to 200 mg, the antibodies (0.5 mg/kg per body weight) were administered intraperitoneally (i.p.) to the tumor-bearing mice on a weekly basis for six weeks, followed by a two-week observation period. In this experiment, the treatment was initiated on Day 11. The volume of the tumor was monitored and recorded using the formula: volume=1/2×length×width×width. Experiments were terminated around days 60-70 after implant. A TGI percent, representing the tumor volume increment of antibody treatments normalized with the tumor volume increment of a non-treatment group (treated with vehicle only) was calculated using the formula: TGI (%)=[1−(Tn−T1)/(Cn−C1)]×100; Tn is the average tumor volume of a treatment group on a given day, T1 is the average tumor of the treatment group on the first day of treatment, Cn is the average tumor volume of the vehicle control group on the same day with Tn, and Cl is the average tumor volume of the vehicle group on the first day of treatment.

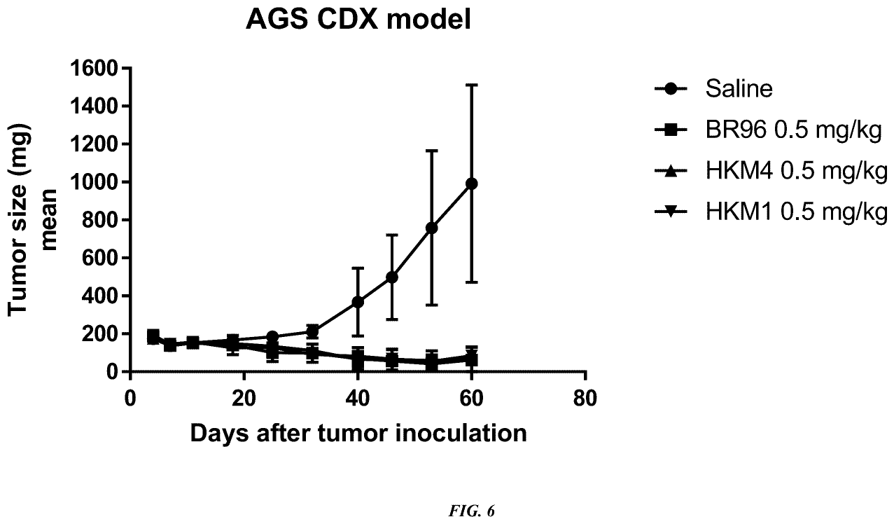

The results (FIG. 6 and table below) show that mice treated with HKM1 or HKM 4 had their tumor size decreased at least after 20 days of tumor inoculation, while the control group of mice (treated with vehicles only) had their tumors continue to grow. The inhibitory effect of HKM1 and HKM4 was similar to the conventional anti-Lewis Y antibody, BR96. When the experiment was terminated on Day 60, the tumor size of the mice treated with the antibodies of the present disclosure was smaller than 1/10 of the tumor size of the control group.

TABLE
Tumor volume reduction and TGI %
of the AGS xenograft tumor model

| Day 60 | Saline (Control) | HKM1 | HKM4 |
|---|---|---|---|
| Tumor volume (mm³) | 991.3 | 85.1 | 66.5 |
| TGI % | — | 108.51% | 110.16% |

### Example 3: Generation of Pro-Antibody (Lock Antibody) and Characterization

The heavy chain and light chain of the conventional anti-Lewis Y antibody BR96 and the antibody of the present disclosure HKM4 were respectively conjugated to a human IgG1 hinge domain (serves as a masking moiety; SEQ ID NO: 27) via a linker having a protease substrate peptide. A cysteine of the human IgG1 domain can form a disulfide bond that makes a steric hindrance to block the antigen-binding site of antibodies BR96 and HKM4. The protease substrate peptide tested in this example included a sequence of GPLGVR (hereinafter “S1”), IPVSLRSG (hereinafter “S2”), or PLGLAG (hereinafter “S3”), which are all Matric-metalloproteinase 2 (MMP2) substrates. Antibodies were expressed via transient transfection of HEK293 or stable transfection of CHO-DG44 and purified using Amsphere™ A3 protein A (JSR) using a similar procedure described in Example 1.

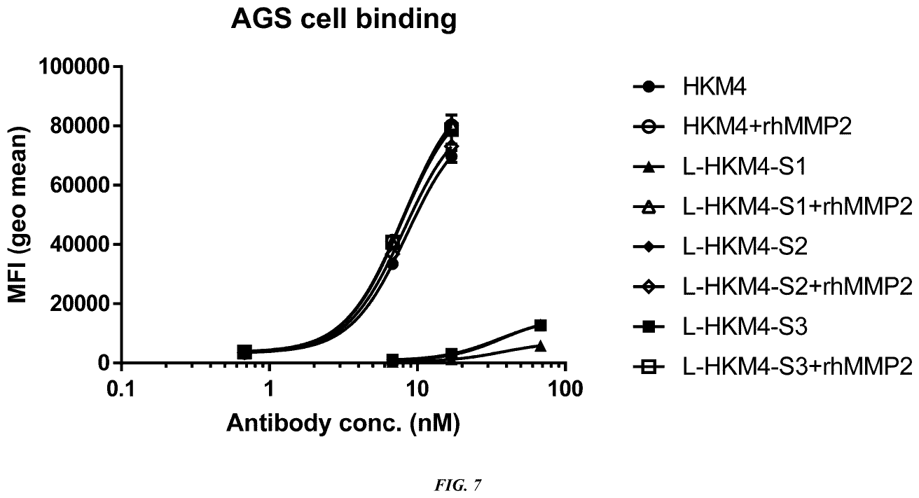

Pro-antibody proteolysis. Experiments were conducted to test the efficiency of the masking moiety in blocking the antigen-binding site of the antibodies and the efficiency of cleaving the linker (the protease substrate peptide) thereby releasing the antibodies from the masking moiety. A cell-binding experiment was performed according to the experiment described in Example 2, while only the AGS cell line was tested here. In the experiments conducted in this example, the pro-antibodies tested were either inactivated or activated by incubation with MMP2 before the experiments. The masking moiety used in the experiment of FIG. 7 comprises the amino acid set forth in SEQ ID NO: 36.

The table below shows the results of the cell-binding data. As shown below, antibodies were either treated with MMP2 (“+”) or not (“−”) before the experiments. The relative activities were calculated using the binding affinity of HKM4 (treated or without treated with MMP2, respectively) as 100%. The results show that without activation (i.e., incubation with MMP2), the pro-antibodies had ignorable binding affinities to the AGS cells. In contrast, after activation, the antibodies were able to bind the cells with great affinities, indicating that the antibodies were released from the masking moieties. Therefore, the blocking efficiency of the masking moieties was reliable, and the proteolysis of the protease substrate peptides of the linkers was efficient. On the other hand, the cell-binding experiment shows consistent results (FIG. 7). The inactivated pro-antibodies barely bound the AGS cells while the activation significantly enabled the binding of the antibodies and the cells.

TABLE
Cell-Binding Data testing the pro-antibody proteolysis

| | | Relative Activity | | | | | | | |
|---|---|---|---|---|---|---|---|---|---|
| | | | | L-HKM4- | | L-HKM4- | | L-HKM4- | |
| | Antibody | HKM4 | | S1 | | S2 | | S3 | |
| | rhMMP2 | − | + | − | + | − | + | − | + |
| Ab | 17.03 | 100% | 100% | 1.8% | 101% | 4% | 91% | 4% | 98% |
| Conc. | 6.81 | 100% | 100% | 1.5% | 99% | 3% | 90% | 3% | 99% |
| (nM) | 0.68 | — | 100% | — | 109% | — | 97% | — | 103% |

O-glycosylation. O-glycosylation is a ubiquitous post-translational modification that takes place in the endoplasmic reticulum and Golgi after a protein is synthesized. The glycans conjugated to a synthesized protein might provide a significant biological role. However, in protein therapeutics production, O-glycosylation can be undesired because, during protein synthesis, the synthesized proteins could have different extents of glycosylation leading to poor homogeneity, which is unfavorable in terms of Chemistry, Manufacturing, and Controls (CMC) management. Therefore, this experiment aimed to control the O-glycosylation of the lock (i.e., the masking moiety) of the pro-antibody of the present disclosure for better homogeneity.

The masking moiety used in the example has the amino acid sequences set forth in SEQ ID NO: 27. It has three O-glycosylation sites at the 4^{th}, 8^{th}, and 10^{th} amino acids. To improve the pro-antibody homogeneity, the three amino acids were replaced with alanine or glycine via point mutation. Briefly, to prepare expression vectors for the O-glycosylation mutation study, HC and LC genes were amplified by elongation PCR method with different sets of PCR primers to produce L-HKM4-L5S1 or L-HKM4-L6S1 genes with T8A or S4A+T8A+T10A, respectively. The PCR product was cloned into pGNX plasmid. Plasmid DNAs were amplified with 400 mL culture in LB broth supplemented with 50 μg/mL Ampicillin and purified using NucleoBond® Xtra Midi Plus kit (Macherey-Nagel).

Then, a reverse-phase liquid chromatography-mass spectrometry (Ultimate 3000RSLCnano system) was performed to examine the extent of O-glycosylation. Briefly, samples were treated with PNGaseF at 37° C. and shaken overnight. Then, the samples were separated using a reversed-phase column (Proswift RP-10). Subsequently, mass spectrometry data was acquired in ESI+ mode using a mass range of 1800-5000 m/z and deconvoluted using Protein Deconvolution Software to determine the masses of the resulting antibodies. Percentage of O-glycosylation was then calculated by dividing the antibody modification intensity with the total intensity.

The results of the O-glycosylation examination are shown in the table below. Replacing the 4^{th}, 8^{th}, and 10^{th} amino acids of the masking moiety with alanine (i.e., L6S1 below) was able to reduce the percentage of O-glycosylation to only 1/10 compared to the original masking moiety. Furthermore, it was surprising to observe that even though the 8^{th} amino acid was determined to have the highest glycosylation rate, replacing the 8^{th} amino acid (i.e., L5S1 below) did not reduce but increased the O-glycosylation significantly.

TABLE
O-glycosylation

| Lock | L1S1 | L5S1 | L6S1 |
|---|---|---|---|
| Lock Sequence | 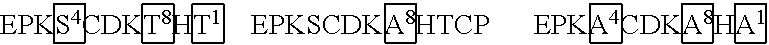 | | |
| | 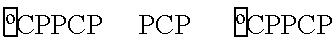 | | |
| | SEQ ID NO: 27 | SEQ ID NO: 35 | SEQ ID NO: 28 |
| Substrate | GPLGVR (S1) | GPLGVR (S1) | GPLGVR (S1) |
| | SEQ ID NO: 32 | SEQ ID NO: 32 | SEQ ID NO: 32 |
| Linker | GGSGPLGVRG | GGSGPLGVRG | GGSGPLGVRG |
| | GGGS | GGGS | GGGS |
| | SEQ ID NO: 29 | SEQ ID NO: 29 | SEQ ID NO: 29 |
| O-glycosylation | 10.9% | 34.8% | 1.2% |

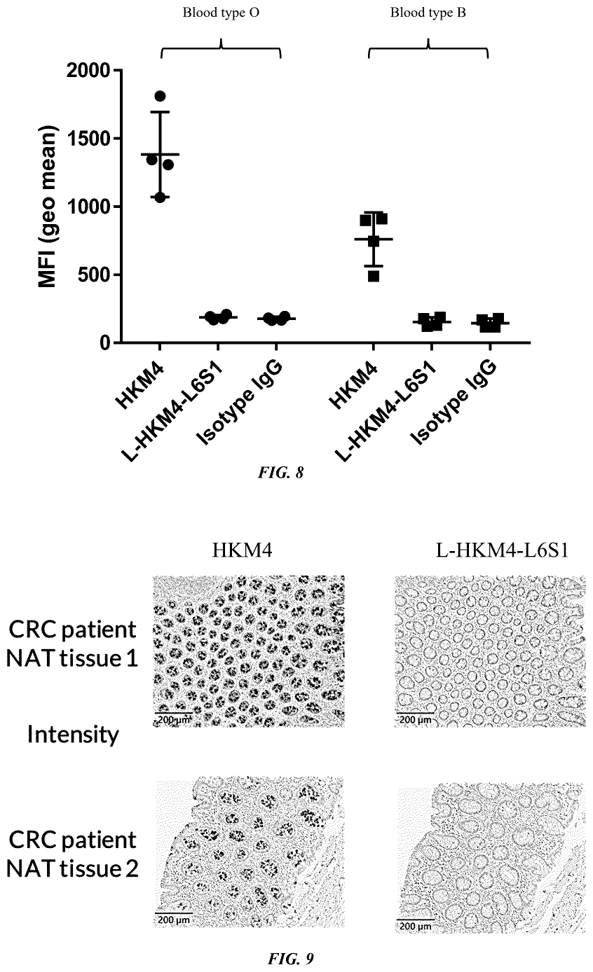

Red blood cell binding. The exemplary pro-antibody's binding affinity to red blood cells was also examined. The experiment was performed according to Example 1, testing an exemplary antibody having HKM4 as the antigen-binding moiety and L6S1 as the masking moiety and the linker, given its desired O-glycosylation percentage. The results (see table below and FIG. 8) show that although the HKM4 antibody already exhibits a lower red blood cell binding affinity, conjugating with the masking moiety can further lower the binding affinity significantly to a similar level as the negative control. This data confirms that the pro-antibody design of the present disclosure can efficiently prevent non-specific binding and reduce the risk of anemia in clinical uses.

TABLE
Red Blood Cell (Blood Type O or Blood Type
B) Binding Assay (data shown in MFI)

| | | L-HKM4- | |
|---|---|---|---|
| Antibody | HKM4 | L6S1 | Isotype IgG |
| Blood Type O | 1382.25 ± 311.09 | 187.50 ± 173.39 | 177.50 ± 13.10 |
| Blood Type B | 761.00 ± 196.18 | 154.25 ± 33.57 | 144.50 ± 34.31 |

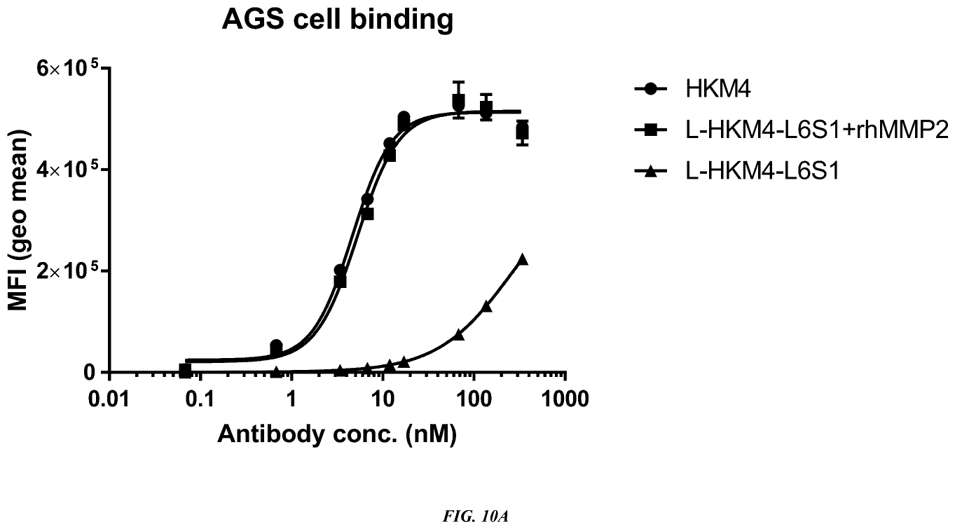

Immunohistochemistry. Next, the exemplary pro-antibody's binding affinity to normal tissues was examined. This experiment was performed as described in Example 2 using a Multiple organ normal tissue microarray (Biomax, FDA999x). The results are shown in the table below. It was observed that HKM4 antibody bound strongly in the digestive tract and breast and moderately in the tonsil, cervix, hypophysis, and thymus gland. The pro-antibody configuration of the present disclosure can help reduce the binding of HKM4 to most normal tissues. Although weak to moderate binding was still observed in the salivary gland and stomach, the L-HKM4-L6S1 only showed a low-intensity signal in two colorectal cancer patients' normal tissue adjacent (NAT) to the tumor (See FIG. 9). This is a favorable safety profile for cancer treatment as it minimizes the binding of HKM4 to normal tissues.

TABLE
Results of the Multiple-normal human tissue array

| Normal Tissue | HKM4 | L-HKM4-L6S1 |
|---|---|---|
| Larynx | 100% | 50% |
| Salivary gland | 100% | 100% |
| Tonsil | 50% | 0% |
| Esophagus | 33% | 0% |
| Stomach | 100% | 100% |
| Small Intestine | 67% | 67% |
| Pancreas | 100% | 0% |
| Breast | 67% | 33% |
| Cervix | 100% | 0% |
| Hypophysis | 67% | 0% |
| Thymus gland | 100% | 0% |

### Example 4: Binding Affinity of the Exemplary Antibody and Pro-Antibody of the Present Disclosure

The kinetics of the binding affinity of the exemplary antibody and pro-antibody to Lewis Y-Gal pentaose antigens were tested in this experiment using surface plasmon resonance (Biacore T200, Cytiva). Biotinylated antigen was immobilized on a Series S SA sensor chip (Cytiva). The concentration of HKM4 were tested at 250-4000 nM, while L-HKM4-L1S1 (pro-HKM4 antibody) were tested at 25-400 nM with a flow rate of 30 μL/min and assessed association for 60 sec and dissociation for 90 sec. Kinetic parameters were determined using the 1:1 Langmuir binding fitting model. The results are shown in the following table.

TABLE
Binding Kinetics

| | | | | Relative | | |
|---|---|---|---|---|---|---|
| | | | | affinity % | | |
| | Ka (SE) | Kd (SE) | K_{D} | (compared | Rmax | Chi² |
| | (1/Ms) | (1/s) | (M) | by K_{D}) | (RU) | (RU²) |
| BR96 | 6.842E+04 | 2.91E−02 | 4.255E−07 | 100.0 | 109 | 2.32 |
| L-BR96* | 4.881E+03 | 1.17E−01 | 2.397E−05 | 1.8 | 33 | 0.03 |
| Act-L-BR96 | 4.750E+05 | 2.59E−01 | 5.459E−07 | 78.0 | 86 | 0.72 |
| HKM4 | 3.601E+04 | 2.97E−01 | 8.260E−06 | 100.0 | 167 | 0.34 |
| L-HKM4-L1S1* | 2.371E+03 | 8.85E−01 | 3.731E−04 | 2.2 | 1241 | 0.01 |
| Act-L-HKM4-L1S1 | 2.514E+04 | 1.33E−01 | 5.291E−06 | 156.1 | 90 | 2.34 |

The response of L-BR96 and L-HKM4-L1S1 were weak, and the affinity parameters approached the detection limit and were hard to determine exactly by the software. The result showing the binding of L-BR96 and L-HKM4-L1S1 was greatly reduced due to the mask.

### Example 5: Anti-Tumor Activity of the Pro-Antibody

This example tested some exemplary pro-antibodies of the present disclosure for their tumor cell-binding affinity, tissue type-binding tendency, and anti-tumor activity. The pro-antibodies tested in this example (see table below) include a pro-HKM4 antibody having the HKM4 antibody as the antigen-binding moiety and the L6S1 as the masking moiety and the linker (i.e., L-HKM4-L6S1) and a pro-BR96 antibody having the conventional BR96 antibody as the antigen-binding moiety and the L1S1as the masking moiety and the linker (i.e., L-BR96). Besides, two different blood types were tested in this experiment, and the result verifies that blood types did not affect the reduced binding affinity contributed by the pro-antibody configuration.

TABLE
Exemplary pro-antibodies tested in Example 4

| | pro-HKM4 antibody | pro-BR96 antibody |
|---|---|---|
| Antigen binding moiety | HKM4 | BR96 |
| Masking moiety | L6 (SEQ ID NO: 28) | L1 (SEQ ID NO: 27) |
| Linker | S1 (SEQ ID NO: 29) | S1 (SEQ ID NO: 29) |

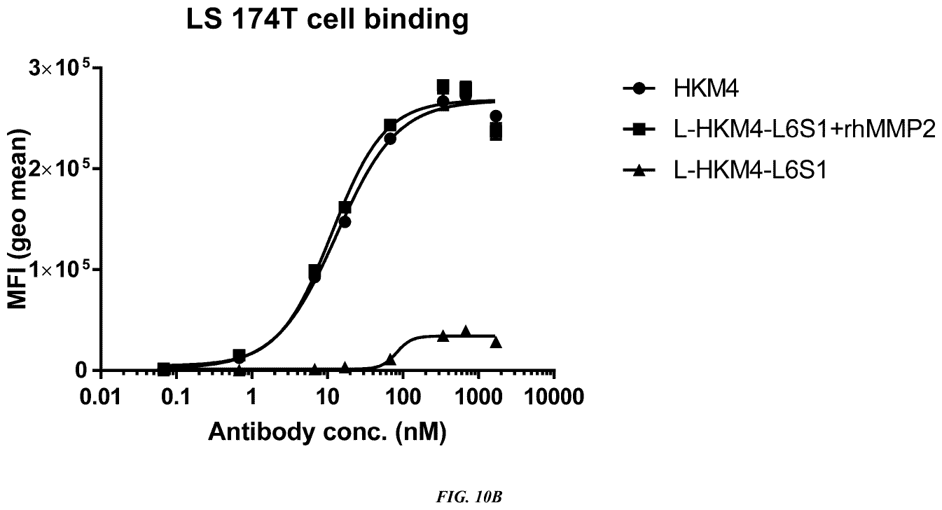

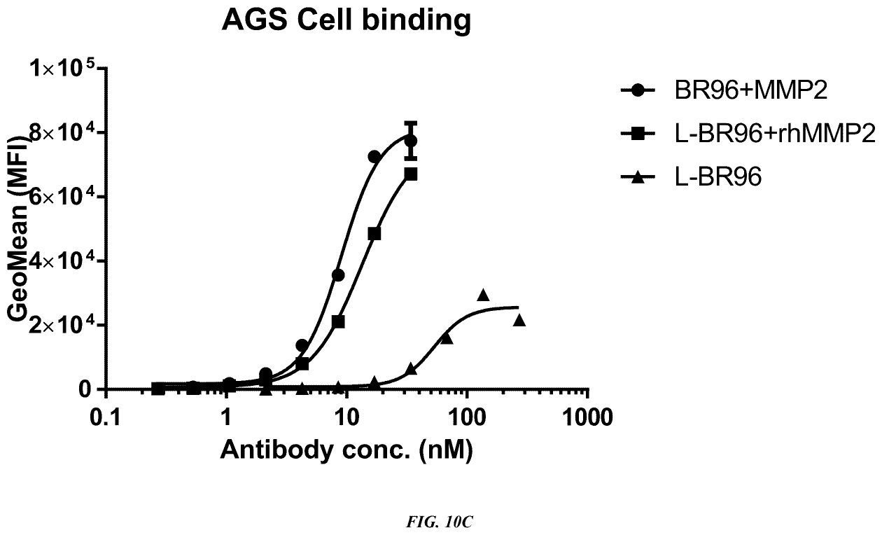

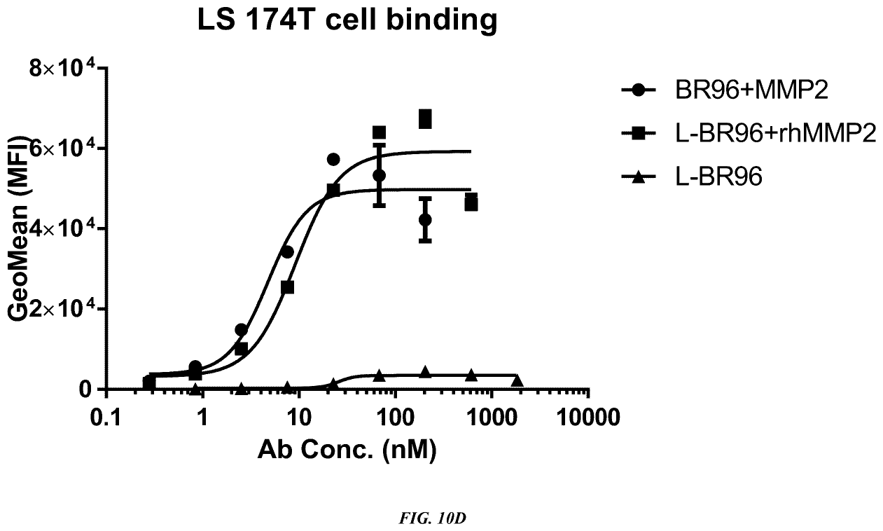

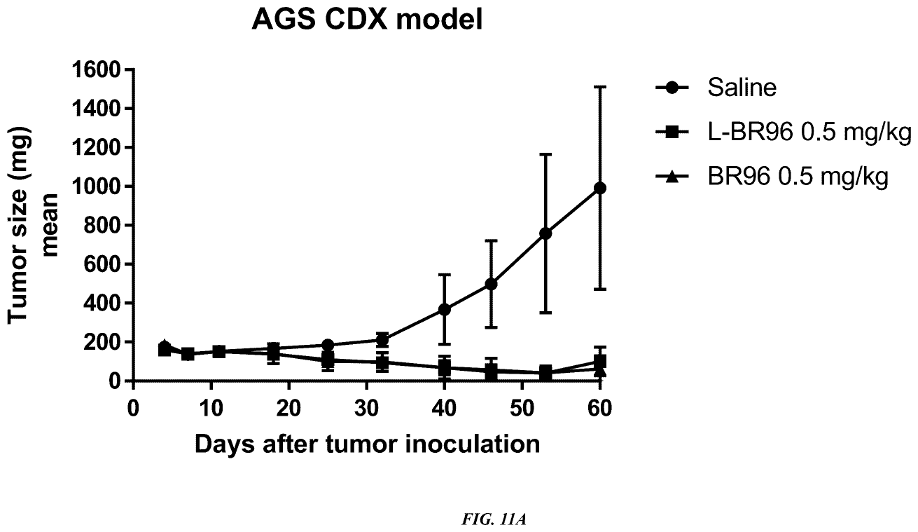

Cancer cell binding. The cancer cell binding affinity was tested according to Example 2 without or with proteolysis, as described in Example 3, to release the antigen-binding moiety from the masking moiety. HKM4 and BR96 without masking moieties were also included in this experiment as positive controls. The results show that both the pro-HKM4 antibody and the pro-BR96 antibody were not able to bind the AGS cells (FIG. 10A and FIG. 10C) and the LS 174T cells (FIG. 10B and FIG. 10D) unless the linkers thereof were cleaved by MMP2 to release the antigen-binding moiety from the masking moiety, thereby activating the HKM4 antibody and the BR96 antibody.

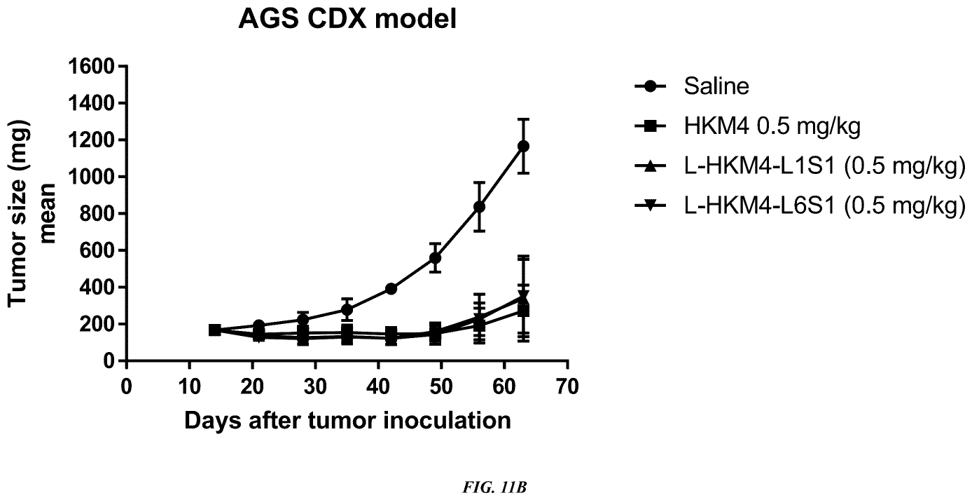

Anti-tumor activity. Next, the anti-tumor activity of the exemplary pro-antibodies of the present disclosure was examined using the AGS xenograft tumor inhibition model as described in Example 2 above. Antibodies and activated pro-antibodies were administered intraperitoneally (i.p., 0.5 mg/kg per body weight) to the tumor-bearing mice on a weekly basis, starting on Day 14 and for six weeks, followed by a two-week observation period. The results were shown in FIG. 11A, FIG. 11B, and the tables below. It was observed that both the activated L-BR96 pro-antibody and the L-HKM4 antibody (L-HKM4-L1S1 and L-HKM4-L6S1) were able to reduce the tumor volume to a similar level as its unlocked counterpart antibody. It was also observed that L-BR96 had a slightly lower inhibitory effect than BR96. Similarly, the inhibitory effect of L-HKM4 was also slightly lower than that of HKM4. This observation suggested that the pro-antibody configuration might slightly affect the inhibitory efficacy because the antibody must be activated first to exert the inhibitory effect. Nevertheless, the pro-antibody is believed to have better overall benefits as the configuration provides better specificity with only a slight compensation of the efficacy. Besides, the L-HKM4-L1S1 pro-antibody and the L-HKM4-L6S1 pro-antibody showed similar levels of tumor volume reduction. This observation suggests that the modification of masking moiety to reduce O-glycosylation did not substantially affect the anti-tumor activity of the pro-antibodies.

TABLE
Tumor volume reduction and TGI % (BR96 and L-BR96).

| Day 60 | Saline | BR96 | L-BR96 |
|---|---|---|---|
| Tumor volume (mm³) | 991.3 | 60.9 | 100.6 |
| TGI % | — | 110.77% | 106.11% |

TABLE
Tumor volume reduction and TGI % (HKM4 and L-HKM4).

| | | | L-HKM4- | L-HKM4- |
|---|---|---|---|---|
| Day 63 | Saline | HKM4 | L1S1 | L6S1 |
| Tumor volume (mm³) | 1165.7 | 271.0 | 338.5 | 351.1 |
| TGI % | — | 89.66% | 82.88% | 81.61% |

### Example 6: Exemplary Antibody-Drug Conjugate and Tumor Inhibitory Effects

This experiment prepared an exemplary antibody-drug conjugate having a pro-antibody L-HKM4-L6S1 according to an example of the present disclosure. The exemplary antibody-drug conjugate was then tested for its ability to inhibit tumors.

Preparation of antibody-drug conjugate. To synthesize the pro-antibody drug conjugate, the pro-antibody L-HKM4-L6S1, according to an example of the present disclosure, was used to conjugate with 7-Ethyl-10-hydroxycamptothecin (SN38) using PerKit™ Antibody SN38 Conjugation Kit (CellMosaic). The conjugation was performed following the manual. Briefly, the pro-antibody L-HKM4-L6S1 was added to the O-succinyl SN38 NHS ester solution and mixed on the rotor at 25° C. for 2 hours. Then, the L-HKM4-L6S1-SN38 was purified by the desalting column to remove the free SN38. The concentration of L-HKM4-L6S1-SN38 was measured by Pierce™ BCA Protein Assay Kit (Thermo). The Drug to antibody (DAR) ratio was measured as 3 (2.67) and purity was characterized by SEC-HPLC.

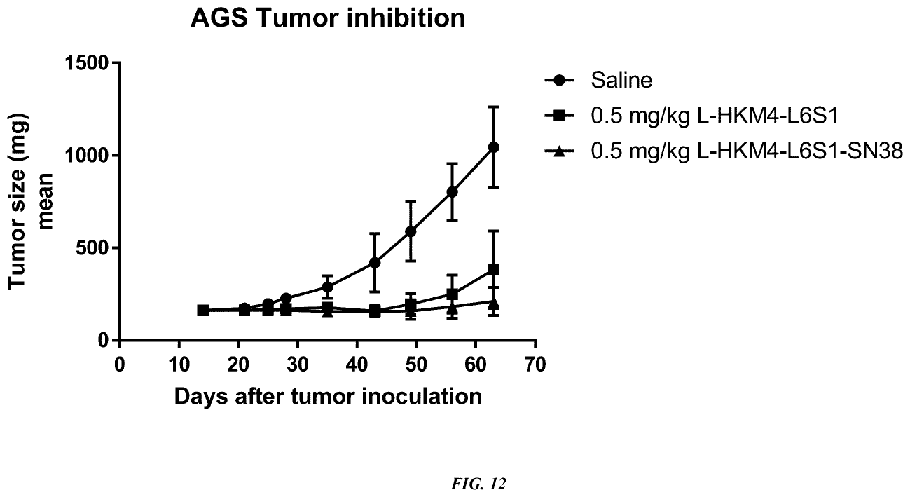

Anti-tumor activity. The anti-tumor activity was examined using the AGS xenograft model as described in Example 1 except that it was the L-HKM4-L6S1-SN38 tested in this experiment. The results were shown in FIG. 12 and the Table below. It was observed, as expected, that L-HKM4-L6S1 was able to reduce the tumor volume significantly. Noticeably, the L-HKM4-L6S1-SN38 showed an even better inhibitory effect than L-HKM4. The inhibitory activities of L-HKM4-L6S1 and L-HKM4-L6S1-SN38 both started to show after Day 20 and reflected in tumor size reduction.

TABLE
Tumor volume reduction and TGI % (L-
HKM4 and L-HKM4-L6S1-drug conjugate).

| | | | L-HKM4-L6S1- |
|---|---|---|---|
| Day 63 | Saline | L-HKM4-L6S1 | SN38 |
| Tumor volume (mm³) | 1044.7 | 337.4 | 189.5 |
| TGI % | — | 80% | 97% |

### Example 7: Linker Selection

This experiment was performed to select a suitable linker for the pro-antibody of the present disclosure. Without wishing to be bound by theory, the present disclosure contemplated that a suitable linker should not adversely affect the productivity of the pro-antibody and quality of the production thereof. On top of that, the determination of a suitable linker also factored in whether a pro-antibody having a candidate linker demonstrates better shielding (blocking efficiency), activation efficiency, or therapeutic efficacy.

The candidate linkers tested in the example are listed below. The candidate linkers have the same substrate sequence (SEQ ID NO: 32) and the same 2^{nd} linkage moiety (SEQ ID NO: 39) while differ in the sequence of the 1^{st} linkage moiety. L1S1 linker and L6S1 linker have a Gly-Gly-Ser (GGS) sequence in their 1^{st} linkage moiety, and the LO7 linker further has an PLAQG sequence (SEQ ID NO: 38). As a result, L1S1 and L6S1 both have 14 amino acids, and LO7 has 19 amino acids. All pro-antibody samples were made using the HKM4 antibody and the same masking moiety of SEQ ID NO: 35, as described herein.

| | Linker | | |
|---|---|---|---|
| Samples | 1^{st} Linkage | | 2^{nd} Linkage |
| Name | moiety | Substrate | moiety |
| L-HKM4-L1S1 | GGS | GPLGVR | GGGGS |
| L-HKM4-L6S1 | GGS | GPLGVR | GGGGS |
| L-HKM4-LO7 | GGSPLAQG | GPLGVR | GGGGS |

Blocking Efficiency. The antigen-binding residues of the exemplary pro-antibodies tested in this experiment were determined by using computer modeling, as described in Huang et al., Chem. Sci., 2021, 12, 9759, herein incorporated by reference in their entirety, which establishes an antigen-containing co-crystal structure. The blocking efficiency, or “cover rate” of was calculated from the blocking frequencies of a CDR residue. A residue was deemed covered if any atom of the masking moiety reaches the CDR residues. The determination was described in Chem. Sci., 2021, 12, 9759, which is herein incorporated by reference in their entirety. According to the computer modeling, L-HKM4-L6S1 demonstrated a cover rate of about 48.90%, and L-HKM4-LO7's cover rate was about 57.60%. This result showed that the LO7 linker had better antigen coverage resulting in better blocking efficiency. The result also suggests that while the masking moiety is designed to reduce off-target binding of the HKM4 antibody, linkers also participate in this mechanism, and the LO7 linker is better than the L6S1 linker in this respect.

### Monomer.

Antibodies L-HKM4-L6S1 and L-HKM4-LO7 were produced using HEK293 cells and subsequently purified by Amsphere™ A3 ProA resin. The purity and aggregation profiles of the antibodies were analyzed using Dionex UltiMate™ 3000 HPLC (Thermo Fisher Scientific) equipped with a SEC-HPLC analytical column (Tosoh Bioscience). The monomer contents of L-HKM4-L6S1 and L-HKM4-LO7 were determined to be 68.88% and 80.47%, respectively. These results confirm that both antibodies were successfully expressed and purified, and that L-HKM4-LO7 exhibited a higher monomer percentage compared to L-HKM4-L6S1, indicating improved molecular stability and purity achieved through the linker optimization.

| Samples Name | Monomer % |
|---|---|
| L-HKM4-L6S1 | 68.88 |
| L-HKM4-LO7 | 80.47 |

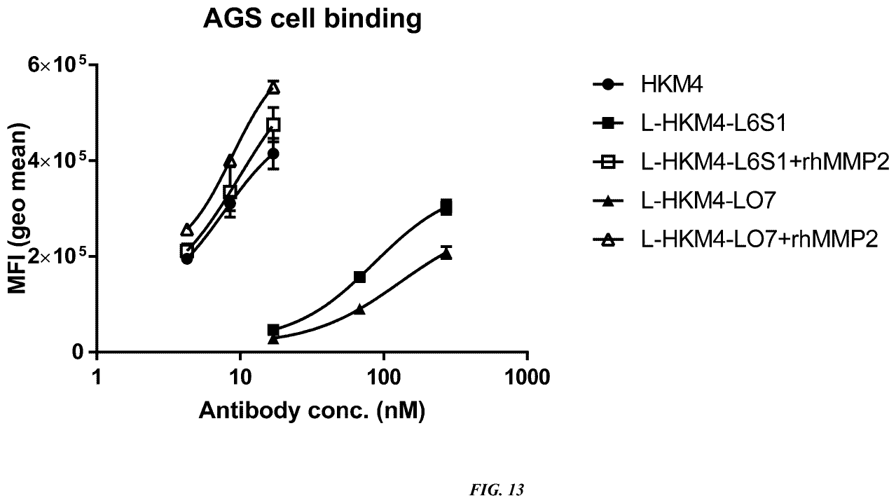

Cancer cell binding. The cancer cell binding affinity was tested according to Example 2 without or with proteolysis, as described in Example 3, to release the antigen-binding moiety from the masking moiety. The cell binding assay (FIG. 13) showed that both pro-antibodies (L-HKM4-L6S1 and L-HKM4-LO7) had weak binding to the AGS cells and demonstrated significant increase in binding after activation, confirming that the masking mechanism was taking effects. It is especially noticeable that, before activation, L-HKM4-LO7 had a weaker binding than that of the L-HKM4-L6S1. This finding suggests that using LO7 as the linker connecting the masking moiety to the antibody provides better blocking efficiency, which is consistent with the computer modeling result described above.

In situ zymography analysis. Next, the exemplary pro-antibodies were tested for their activation efficiency using real cancer patients' tissue samples. In this experiment, exemplary antibodies and pro-antibodies were conjugated to Alexa Fluor 647 dye (Abcam, ab269823) through a reaction targeting primary amine groups on lysine residues and the N-terminal amine of the protein, in accordance with the manufacturer's protocol. Frozen gastric tumor tissue samples, from three individual patients (#22, #47, and #64) were embedded in OCT compound. Five-micrometers of frozen tissue sections were cut using a cryostat (LEICA) and mounted on glass slides. For staining, 100 μL of a solution containing 13.6 nM Alexa Fluor 647-labeled L-HKM4-L6S1, L-HKM4-LO7 or HKM4 in Tris buffer containing BSA was applied to each section and incubated at 37° C. with shaking at 50 rpm for 48 hours. Protease inhibition was performed using 100 μL of a 1:100 dilution of a broad-spectrum protease inhibitor cocktail set III (539134, EMD Millipore, Billerica, MA) combined with 600 mM EDTA in Tris buffer. Fluorescent signals were acquired, and image analysis was performed using TissueFAXS and TissueQuest (TissueGnostics, Vienna, Austria).

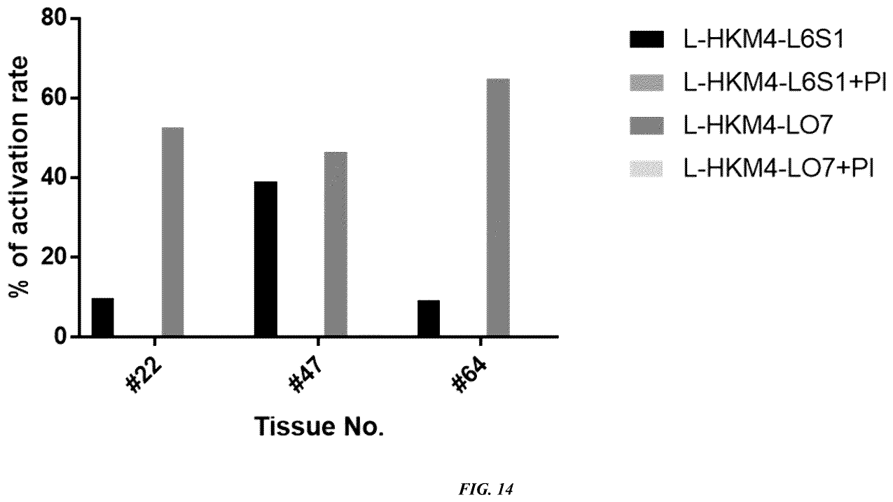

The result showed that pro-antibody L-HKM4-LO7 of the present disclosure exhibited superior activation levels across multiple tissue samples compared with L-HKM4-L6S1. See FIG. 14 and the table below, L-HKM4-LO7's activation rate in patient #22 tissue was about 52.64%, and L-HKM4-L6S1's activation rate was about 9.7%. Similarly, in patient #47 tissue and patient #64 tissue, L-HKM4-LO7 had an activation rate of about 46.58% and about 64.83% respectively, and L-HKM4-L6S1's activation rate was about 39.13% and 9.35% respectively. The activation was triggered by proteases existing in tumor sample, which was verified by adding protease inhibitors (PI). These results demonstrate that the LO7 linker enabled more efficient protease-mediated unlocking, which is expected to translate into better therapeutic efficacy.

| | | L-HKM4- | | L-HKM4- |
|---|---|---|---|---|
| Tissue No. | L-HKM4-L6S1 | L6S1 + PI | L-HKM4-LO7 | LO7 + PI |
| #22 | 9.7% | 0.02% | 52.64% | 0.05% |
| #47 | 39.13% | 0.24% | 46.58% | 0.71% |
| #64 | 9.35% | 0.08% | 64.83% | 0.02% |

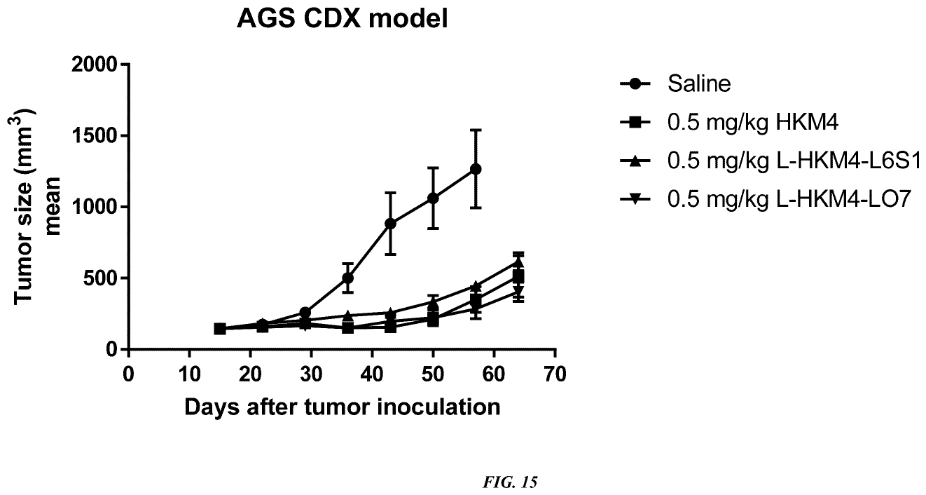

Tumor inhibitory effects. Lastly, the exemplary pro-antibodies' tumor inhibitory effects were evaluated. The tumor inhibitory effect was examined using the AGS xenograft model as described in Example 1. The results were shown in FIG. 15 and the Table below. No significant tumor growth was observed in mice administrated with HKM4, L-HKM4-L6S1, or L-HKM4-LO7 until around Day 50 after administration. In contrast, the mice of negative control group experienced tumor growth as early as Day 30. It was also observed that the tumor of the L-HKM4-L6S1 group grew bigger than that of the L-HKM4-LO7 group. On Day 57, the TGI % of the L-HKM4-L6S1 group was 73%, the TGI of the HKM group was 82%, and that of the L-HKM4-LO7 group was 88%. That said, L-HKM4-L6S1 was able to reduce the tumor volume significantly while L-HKM4-LO7 performed even better.

### OTHER EMBODIMENTS

Embodiment 1. An isolated antibody or an antigen-binding fragment thereof, comprising: (a) a heavy chain variable region (VH) comprising a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3; and (b) a light chain variable region (VL) comprising a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

Embodiment 2. The isolated antibody or the antigen-binding fragment thereof of Embodiment 1, wherein that the 4^{th} amino acid of the VH CDR1 is Isoleucine (I), the 5^{th} amino acid of the VH CDR2 is Aspartic acid (D), the 12^{th} amino acid of the VH CDR2 is Glutamine (Q), the 15^{th} amino acid of the VH CDR2 is Leucine (L), the 4^{th} amino acid of the VH CDR3 is Serine(S), the 5^{th} amino acid of the VH CDR3 is Glutamic acid (E), the 5^{th} amino acid of the VL CDR1 is Asparagine (N), and/or the 9^{th} amino acid of the VL CDR1 is Threonine (T).

Embodiment 3. The isolated antibody or the antigen-binding fragment thereof of Embodiment 1 or Embodiment 2, wherein the VH comprises an amino acid sequence having at least 90%, 91%, 92%, 93%, 94%, 95%, 96%, 97%, 98%, or 99% percent identity to SEQ ID NO: 4 or SEQ ID NO: 15.

Embodiment 4. The isolated antibody or the antigen-binding fragment thereof of Embodiment 3, wherein the 3^{rd} amino acid of the VH is Glutamine (Q), the 19^{th} amino acid of the VH is Arginine (R), the 20^{th} amino acid of the VH is Leucine (L), the 23^{rd} amino acid of the VH is Alanine (A), the 40^{th} amino acid of the VH is Alanine (A), the 84^{th} amino acid of the VH is Asparagine (N), the 85^{th} amino acid of the VH is Serine(S), the 87^{th} amino acid of the VH is Arginine (R), the 88^{th} amino acid of the VH is Alanine (A), and/or the 93^{rd} amino acid of the VH is Valine (V).

Embodiment 5. The isolated antibody or the antigen-binding fragment thereof of Embodiment 3 or Embodiment 4, wherein the VH is encoded by a sequence, having at least 70%, 75%, 80%, 85%, 90%, 91%, 92%, 93%, 94%, 95%, 96%, 97%, 98%, or 99% percent identity to SEQ ID NO: 6.

Embodiment 6. The isolated antibody or the antigen-binding fragment thereof of any one of Embodiments 1 to 5, wherein the VL comprises an amino acid sequence having at least 90%, 91%, 92%, 93%, 94%, 95%, 96%, 97%, 98%, or 99% percent identity to SEQ ID NO: 11.

Embodiment 7. The isolated antibody or the antigen-binding fragment thereof of Embodiment 6, wherein the 2^{nd} amino acid of the VL is Isoleucine (I), the 3^{rd} amino acid of the VL is Valine (V), the 7^{th} amino acid of the VL is Threonine (T), the 14^{th} amino acid of the VL is Threonine (T), the 17^{th} amino acid of the VL is Glutamine (Q), the 18^{th} amino acid of the VL is Proline (P), the 88^{th} amino acid of the VL is Valine (V), the 105^{th} amino acid of the VL is Proline (P), the 109^{th} amino acid of the VL is Valine (V), and/or the 110^{th} amino acid of the VL is Aspartic acid (D).

Embodiment 8. The isolated antibody or the antigen-binding fragment thereof of Embodiment 6 or Embodiment 7, wherein the VL is encoded by a sequence, having at least 70%, 75%, 80%, 85%, 90%, 91%, 92%, 93%, 94%, 95%, 96%, 97%, 98%, or 99% percent identity to SEQ ID NO: 13.

Embodiment 9. The isolated antibody or the antigen-binding fragment thereof of any one of Embodiments 1 to 8, comprising a heavy chain comprising an amino acid sequence having at least 90%, 91%, 92%, 93%, 94%, 95%, 96%, 97%, 98%, or 99% percent identity to SEQ ID NO: 5.

Embodiment 10. The isolated antibody or the antigen-binding fragment thereof of Embodiment 9, wherein the heavy chain is encoded by a sequence, having at least 70%, 75%, 80%, 85%, 90%, 91%, 92%, 93%, 94%, 95%, 96%, 97%, 98%, or 99% percent identity to SEQ ID NO: 7.

Embodiment 11. The isolated antibody or the antigen-binding fragment thereof of any one of Embodiments 1 to 10, comprising a light chain comprising an amino acid sequence having at least 90%, 91%, 92%, 93%, 94%, 95%, 96%, 97%, 98%, or 99% percent identity to SEQ ID NO: 12.

Embodiment 12. The isolated antibody or the antigen-binding fragment thereof of Embodiment 11, wherein the light chain is encoded by a sequence, having at least 70%, 75%, 80%, 85%, 90%, 91%, 92%, 93%, 94%, 95%, 96%, 97%, 98%, or 99% percent identity to SEQ ID NO: 14.

Embodiment 13. The isolated antibody or the antigen-binding fragment thereof of any one of Embodiments 1 to 12, wherein the antibody is a monoclonal antibody.

Embodiment 14. The isolated antibody or the antigen-binding fragment thereof of any one of Embodiments 1 to 13, wherein the antibody is a humanized antibody.

Embodiment 15. The isolated antibody or the antigen-binding fragment thereof of any one of Embodiments 1 to 14, wherein the antigen-binding fragment is selected from a group consisting of a Fab fragment, an F(ab′)2 fragment, an Fv fragment, a single chain Fv (ScFv) antibody, a diabody, a minibody, a nanobody (VHH), and a linear antibody.

Embodiment 16. The isolated antibody or the antigen-binding fragment thereof of any one of Embodiments 1 to 15, being capable of binding specifically to a Lewis Y antigen.

Embodiment 17. The isolated antibody or the antigen-binding fragment thereof of any one of Embodiments 1 to 16, wherein antibody or the antigen-binding fragment thereof does not bind Lewis X (LeX), Lewis A (LeA), Lewis B (LeB), or H type 2 antigen.

Embodiment 18. An isolated antibody or an antigen-binding fragment thereof comprising: a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15; a light chain variable region (VL) comprising an amino acid sequence having at least 90%, 91%, 92%, 93%, 94%, 95%, 96%, 97%, 98%, or 99% percent identity to SEQ ID NO: 11.

Embodiment 19. The isolated antibody or the antigen-binding fragment thereof of Embodiment 18, wherein the 3^{rd} amino acid of the VH is Glutamine (Q), the 19^{th} amino acid of the VH is Arginine (R), the 20^{th} amino acid of the VH is Leucine (L), the 23^{rd} amino acid of the VH is Alanine (A), the 40^{th} amino acid of the VH is Alanine (A), the 84^{th} amino acid of the VH is Asparagine (N), the 85^{th} amino acid of the VH is Serine(S), the 87^{th} amino acid of the VH is Alanine (A), the 88^{th} amino acid of the VH is Alanine (A), and/or the 93^{rd} amino acid of the CH is Valine (V).

Embodiment 20. The isolated antibody or the antigen-binding fragment thereof of Embodiment 18 or Embodiment 19, wherein the VH is encoded by a sequence, having at least 70%, 75%, 80%, 85%, 90%, 91%, 92%, 93%, 94%, 95%, 96%, 97%, 98%, or 99% percent identity to SEQ ID NO: 6.

Embodiment 21. The isolated antibody or the antigen-binding fragment thereof of any one of Embodiments 18 to 20, wherein the 2^{nd} amino acid of the VL is Isoleucine (I), the 3^{rd} amino acid of the VL is Valine (V), the 7^{th} amino acid of the VL is Threonine (T), the 14^{th} amino acid of the VL is Threonine (T), the 17^{th} amino acid of the VL is Glutamine (Q), the 18^{th} amino acid of the VL is Proline (P), the 88^{th} amino acid of the VL is Valine (V), the 105th amino acid of the VL is Proline (P), the 109^{th} amino acid of the VL is Valine (V), and/or the 110th amino acid of the VL is Aspartic acid (D).

Embodiment 22. The isolated antibody or the antigen-binding fragment thereof of any one of Embodiments 18 to 21, wherein the VL is encoded by a sequence, having at least 70%, 75%, 80%, 85%, 90%, 91%, 92%, 93%, 94%, 95%, 96%, 97%, 98%, or 99% percent identity to SEQ ID NO: 13.

Embodiment 23. The isolated antibody or the antigen-binding fragment thereof of any one of Embodiments 18 to 22, wherein the antibody is monoclonal.

Embodiment 24. The isolated antibody or the antigen-binding fragment thereof of any one of Embodiments 18 to 23, wherein the antibody is a humanized antibody.

Embodiment 25. The isolated antibody or the antigen-binding fragment thereof of any one of Embodiments 18 to 24, wherein the antigen-binding fragment is selected from a group consisting of a Fab fragment, an F(ab′)2 fragment, an Fv fragment, a single chain Fv (ScFv) antibody, a diabody, a minibody, a nanobody (VHH), and a linear antibody.

Embodiment 26. An isolated nucleic acid encoding the isolated antibody or the antigen-binding fragment thereof of any one of Embodiments 1 to 25.

Embodiment 27. The isolated nucleic acid of Embodiment 26, comprising a first polynucleotide encoding a VH of the isolated antibody or the antigen-binding fragment thereof, wherein the first polynucleotide comprises a sequence having at least 70%, 75%, 80%, 85%, 90%, 91%, 92%, 93%, 94%, 95%, 96%, 97%, 98%, or 99% percent identity to SEQ ID NO: 6.

Embodiment 28. The isolated nucleic acid of Embodiment 26 or Embodiment 27, comprising a second polynucleotide encoding a heavy chain of the isolated antibody or the antigen-binding fragment thereof, wherein the second polynucleotide comprises a sequence having at least 70%, 75%, 80%, 85%, 90%, 91%, 92%, 93%, 94%, 95%, 96%, 97%, 98%, or 99% percent identity to SEQ ID NO: 7.

Embodiment 29. The isolated nucleic acid of any one of Embodiments 26 to 28, comprising a third polynucleotide encoding a VL of the isolated antibody or the antigen-binding fragment thereof, wherein the third polynucleotide comprises a sequence having at least 70%, 75%, 80%, 85%, 90%, 91%, 92%, 93%, 94%, 95%, 96%, 97%, 98%, or 99% percent identity to SEQ ID NO: 13.

Embodiment 30. The isolated nucleic acid of any one of Embodiments 26 to 29, comprising a fourth polynucleotide encoding a light chain of the isolated antibody or the antigen-binding fragment thereof, wherein the fourth polynucleotide comprises a sequence having at least 70%, 75%, 80%, 85%, 90%, 91%, 92%, 93%, 94%, 95%, 96%, 97%, 98%, or 99% percent identity to SEQ ID NO: 14.

Embodiment 31. A recombinant vector, comprising a nucleic acid encoding the isolated antibody or the antigen-binding fragment thereof of any one of Embodiments 1 to 25, or comprising the isolated nucleic acid of any one of Embodiments 26 to 30.

Embodiment 32. A recombinant vector, comprising a nucleic acid encoding an antigen-binding moiety, comprising: a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3.

Embodiment 33. The recombinant vector of Embodiment 32, wherein the 4th amino acid of the VH CDR1 is Isoleucine (I), the 5^{th} amino acid of the VH CDR2 is Aspartic acid (D), the 12^{th} amino acid of the VH CDR2 is Glutamine (Q), the 15^{th} amino acid of the VH CDR2 is Leucine (L), the 4^{th} amino acid of the VH CDR3 is Serine(S), and/or the 5^{th} amino acid of the VH CDR3 is Glutamic acid (E).

Embodiment 34. The recombinant vector of Embodiment 32 or Embodiment 33, wherein the antigen-binding moiety comprises an amino acid sequence having at least 90%, 91%, 92%, 93%, 94%, 95%, 96%, 97%, 98%, or 99% percent identity to SEQ ID NO: 4 or SEQ ID NO: 15.

Embodiment 35. The recombinant vector of Embodiment 34, wherein the 3rd amino acid of the antigen-binding moiety is Glutamine (Q), the 19^{th} amino acid of the VH is Arginine (R), the 20^{th} amino acid of the VH is Leucine (L), the 23^{rd} amino acid of the VH is Alanine (A), the 40^{th} amino acid of the VH is Alanine (A), the 84^{th} amino acid of the VH is Asparagine (N), the 85^{th} amino acid of the VH is Serine(S), the 87^{th} amino acid of the VH is Alanine (A), the 88^{th} amino acid of the VH is Alanine (A), and/or the 93^{rd} amino acid of the CH is Valine (V).

Embodiment 36. A recombinant vector, comprising a nucleic acid encoding a light chain variable region (VL) comprising: a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

Embodiment 37. The recombinant vector of Embodiment 36, provided that the 5^{th} amino acid of the VL CDR1 is Asparagine (N) and/or the 5^{th} amino acid of the VL CDR1 is Threonine (T).

Embodiment 38. The recombinant vector of Embodiment 36 or Embodiment 37, wherein the VL comprises an amino acid sequence having at least 90%, 91%, 92%, 93%, 94%, 95%, 96%, 97%, 98%, or 99% percent identity to SEQ ID NO: 11.

Embodiment 39. The recombinant vector of Embodiment 38, wherein the 2nd amino acid of the VL is Isoleucine (I), the 3^{rd} amino acid of the VL is Valine (V), the 7^{th} amino acid of the VL is Threonine (T), the 14^{th} amino acid of the VL is Threonine (T), the 17^{th} amino acid of the VL is Glutamine (Q), the 18^{th} amino acid of the VL is Proline (P), the 88^{th} amino acid of the VL is Valine (V), the 105^{th} amino acid of the VL is Proline (P), the 109^{th} amino acid of the VL is Valine (V), and/or the 110^{th} amino acid of the VL is Aspartic acid (D).

Embodiment 40. A pro-antibody, comprising an antigen-binding moiety and a masking moiety linking to the antigen-binding moiety via a linker; wherein the antigen-binding moiety is configured to bind a Lewis Y antigen, and the antigen-binding moiety comprises at least one of (i) to (iv) or at least one of (v) and (vi): (i) a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15; (ii) a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1; a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2; a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3; (iii) a light chain variable region (VL) comprising an amino acid sequence having at least 90%, 91%, 92%, 93%, 94%, 95%, 96%, 97%, 98%, or 99% percent identity to SEQ ID NO: 11; (iv) a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8; a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10; (v) a heavy chain variable region (VH) comprising a heavy chain complementarity determining region 1 (VH CDR1) comprising an amino acid sequence that has at least 90%, 91%, 92%, 93%, 94%, 95%, 96%, 97%, 98%, or 99% percent identity to the amino acid sequence set forth in SEQ ID NO: 17, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 18, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 19; and a light chain variable region (VL) comprising a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 22, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 23, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 24. (vi) a VH comprising an amino acid sequence having at least 90%, 91%, 92%, 93%, 94%, 95%, 96%, 97%, 98%, or 99% percent identity to the amino acid sequence set forth in SEQ ID NO: 20; and a VL comprising an amino acid sequence having at least 90%, 91%, 92%, 93%, 94%, 95%, 96%, 97%, 98%, or 99% percent identity to SEQ ID NO: 25; the masking moiety is configured to interfere the antigen-binding moiety from binding a Lewis Y antigen, and the masking moiety comprises an amino acid sequence having at least 90%, 91%, 92%, 93%, 94%, 95%, 96%, 97%, 98%, or 99% percent identity to SEQ ID NO: 27.

Embodiment 41. The pro-antibody of Embodiment 40, wherein the antigen-binding moiety is the antibody or the antigen-binding fragment thereof of any one of Embodiments 1 to 25.

Embodiment 42. The pro-antibody of Embodiment 40 or Embodiment 41, wherein the antigen-binding moiety comprises: a VH comprising an amino acid sequence having at least 90%, 91%, 92%, 93%, 94%, 95%, 96%, 97%, 98%, or 99% percent identity to SEQ ID NO: 4; and a VL comprising an amino acid sequence having at least 90%, 91%, 92%, 93%, 94%, 95%, 96%, 97%, 98%, or 99% percent identity to SEQ ID NO: 11.

Embodiment 43. The pro-antibody of Embodiment 40 or Embodiment 41, wherein the antigen-binding moiety comprises: a VH comprising an amino acid sequence having at least 90%, 91%, 92%, 93%, 94%, 95%, 96%, 97%, 98%, or 99% percent identity to SEQ ID NO: 15; and a VL comprising an amino acid sequence having at least 90%, 91%, 92%, 93%, 94%, 95%, 96%, 97%, 98%, or 99% percent identity to SEQ ID NO: 11.

Embodiment 44. The pro-antibody of any one of Embodiments 40 to 43, wherein the masking moiety has at least one Serine(S) or Threonine (T) thereof being replaced with an amino acid other than Serine(S) and Threonine (T).

Embodiment 45. The pro-antibody of Embodiment 44, wherein at least one of the 4^{th}, the 8^{th}, and the 10^{th} amino acids is replaced with the amino acid other than Serine(S) and Threonine (T).

Embodiment 46. The pro-antibody of Embodiment 44 or Embodiment 45, wherein the amino acid other than Serine(S) and Threonine (T) is Alanine (A) or Glycine (G).

Embodiment 47. The pro-antibody of any one of Embodiments 44 to 46, wherein only one of the 4^{th}, the 8^{th}, and the 10^{th} amino acids is replaced with the amino acid other than Serine(S) and Threonine (T), provided that when the 8^{th} amino acid of the linker is Alanine (A), at least one of the 4^{th} and the 10^{th} amino acids is replaced with the amino acid other than Serine(S) and Threonine (T).

Embodiment 48. The pro-antibody of any one of Embodiments 44 to 47, wherein the 4^{th} amino acid, the 8^{th} amino acid, and the 10^{th} amino acid are replaced with Alanine (A).

Embodiment 49. The pro-antibody of Embodiment 48, wherein the masking moiety comprises an amino acid sequence having at least 90%, 91%, 92%, 93%, 94%, 95%, 96%, 97%, 98%, or 99% percent identity to SEQ ID NO: 28.

Embodiment 50. The pro-antibody of any one of Embodiments 40 to 49, wherein the linker comprises a proteinase substrate flanked by a first linkage moiety and a second linkage moiety, and at least one of the first linkage moiety and the second linkage moiety comprises an amino acid sequence of PLAQ (SEQ ID NO: 37) or an amino acid sequence of Gly-Gly-Ser (GGS).

Embodiment 51. The pro-antibody of Embodiment 50, wherein both the first linkage moiety and the second linkage moiety comprise the amino acid sequence of GGS, and at least one of the first linkage moiety and the second linkage moiety comprises an amino acid sequence of PLAQ (SEQ ID NO: 37).

Embodiment 52. The pro-antibody of Embodiment 50 or Embodiment 51, wherein the first linkage moiety connects the masking moiety to the proteinase substrate, and the second linkage moiety connects the proteinase substrate to the antigen-binding moiety.

Embodiment 53. The pro-antibody of any one of Embodiments 50 to 52, wherein the first linkage moiety comprises an amino acid sequence of PLAQG (SEQ ID NO: 38), and the second linkage moiety comprises an amino acid sequence of GGGGS (SEQ ID NO: 39).

Embodiment 54. The pro-antibody of Embodiment 53, wherein the first linkage moiety comprises an amino acid sequence of GGSPLAQG (SEQ ID NO: 40).

Embodiment 55. The pro-antibody of any one of Embodiments 50 to 52, wherein the first linkage moiety comprises the amino acid sequence of GGS, and the second linkage moiety comprises an amino acid sequence of GGGGS (SEQ ID NO: 39).

Embodiment 56. The pro-antibody of any one of Embodiments 50 to 55, wherein the linker is a first linker and the proteinase substrate is a first proteinase substrate, and the pro-antibody further comprises a second linker, and further wherein the first linker connects the masking moiety to a heavy chain of the antigen-binding moiety, and the second linker connects the masking moiety to a light chain of the antigen-binding moiety; and the second linker comprises a second proteinase substrate flanked by a third linkage moiety and a fourth linkage moiety, and at least one of the third linkage moiety and the fourth linkage moiety comprises an amino acid sequence of PLAQ (SEQ ID NO: 37) or an amino acid sequence of GGS.

Embodiment 57. The pro-antibody of any one of Embodiments 40 to 56, wherein the proteinase substrate is cleavable by a matrix metalloproteinase (MMP), urokinase (Upa), matriptase, Factor Xa, legumain, tissue plasminogen activator (tPA), or ADAM metalloproteinases.

Embodiment 58. The pro-antibody of any one of Embodiments 40 to 57, wherein the linker comprises an amino acid sequence having at least 90%, 91%, 92%, 93%, 94%, 95%, 96%, 97%, 98%, or 99% percent identity to SEQ ID NO: 41, SEQ ID NO: 42, SEQ ID NO: 43, SEQ ID NO: 29, SEQ ID NO: 30, or SEQ ID NO: 31.

Embodiment 59. A pro-antibody, comprising an antigen-binding moiety and a masking moiety, wherein the antigen-binding moiety and the masking moiety are linked via a cleavable linker, comprising a proteinase substrate flanked by a first linkage moiety and a second linkage moiety, and at least one of the first linkage moiety and the second linkage moiety comprises an amino acid sequence of PLAQ (SEQ ID NO: 37); wherein the antigen-binding moiety comprises (i) a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15; and a light chain variable region (VL) comprising an amino acid sequence having at least 90%, 91%, 92%, 93%, 94%, 95%, 96%, 97%, 98%, or 99% percent identity to SEQ ID NO: 11; or (ii) a VH comprising an amino acid sequence having at least 90%, 91%, 92%, 93%, 94%, 95%, 96%, 97%, 98%, or 99% percent identity to the amino acid sequence set forth in SEQ ID NO: 20; and a VL comprising an amino acid sequence having at least 90%, 91%, 92%, 93%, 94%, 95%, 96%, 97%, 98%, or 99% percent identity to the amino acid sequence set forth in SEQ ID NO: 25.

Embodiment 60. The pro-antibody of Embodiment 59, wherein at least one of the first linkage moiety and the second linkage moiety comprises an amino acid sequence of GGS.

Embodiment 61. The pro-antibody of Embodiment 59 or Embodiment 60, wherein both the first linkage moiety and the second linkage moiety comprise the amino acid sequence of GGS.

Embodiment 62. The pro-antibody of any one of Embodiments 59 to 61, wherein the first linkage moiety connects the masking moiety to the proteinase substrate, and the second linkage moiety connects the proteinase substrate to the antigen-binding moiety.

Embodiment 63. The pro-antibody of any one of Embodiments 59 to 62, wherein the first linkage moiety comprises an amino acid sequence of PLAQG (SEQ ID NO: 38), and the second linkage moiety comprises an amino acid sequence of GGGGS (SEQ ID NO: 39).

Embodiment 64. The pro-antibody of Embodiment 63, wherein the first linkage moiety comprises an amino acid sequence of GGSPLAQG (SEQ ID NO: 40).

Embodiment 65. The pro-antibody of any one of Embodiments 59 to 64, wherein the linker is a first linker and the proteinase substrate is a first proteinase substrate, and the pro-antibody further comprises a second linker, and further wherein the first linker connects the masking moiety to a heavy chain of the antigen-binding moiety, and the second linker connects the masking moiety to a light chain of the antigen-binding moiety; and the second linker comprises a second proteinase substrate flanked by a third linkage moiety and a fourth linkage moiety, and at least one of the third linkage moiety and the fourth linkage moiety comprises an amino acid sequence of PLAQ (SEQ ID NO: 37).

Embodiment 66. The pro-antibody of Embodiment 65, wherein at least one of the third linkage moiety and the fourth linkage moiety comprises an amino acid sequence of GGS.

Embodiment 67. The pro-antibody of Embodiment 65 or Embodiment 66, wherein both the third linkage moiety and the fourth linkage moiety comprise the amino acid sequence of GGS.

Embodiment 68. The pro-antibody of any one of Embodiments 65 to 67, wherein the third linkage moiety comprises an amino acid sequence of PLAQG (SEQ ID NO: 38), and the fourth linkage moiety comprises an amino acid sequence of GGGGS (SEQ ID NO: 39).

Embodiment 69. The pro-antibody of Embodiment 68, wherein the third linkage moiety comprises an amino acid sequence of GGSPLAQG (SEQ ID NO: 40).

Embodiment 70. The pro-antibody of Embodiments 59 to 69, wherein the masking moiety comprises an amino acid sequence having at least 90%, 91%, 92%, 93%, 94%, 95%, 96%, 97%, 98%, or 99% percent identity to SEQ ID NO: 27.

Embodiment 71. The pro-antibody of Embodiment 70, wherein the masking moiety has at least one of Serine(S) or Threonine (T) thereof being replaced with an amino acid other than Serine(S) and Threonine (T).

Embodiment 72. The pro-antibody of Embodiment 71, wherein at least one of the 4^{th}, the 8^{th}, and the 10^{th} amino acids is replaced with the amino acid other than Serine(S) and Threonine (T).

Embodiment 73. The pro-antibody of Embodiment 71 or Embodiment 72, wherein the amino acid other than Serine(S) and Threonine (T) is Alanine (A) or Glycine (G).

Embodiment 74. The pro-antibody of any one of Embodiments 71 to 73, wherein only one of the 4^{th}, the 8^{th}, and the 10^{th} amino acids is replaced with the amino acid other than Serine(S) and Threonine (T), provided that when the 8^{th} amino acid of the linker is not Alanine (A), at least one of the 4^{th} and the 10^{th} amino acids is replaced with the amino acid other than Serine(S) and Threonine (T).

Embodiment 75. The pro-antibody of any one of Embodiments 71 to 74, wherein the 4^{th} amino acid, the 8^{th} amino acid, and the 10^{th} amino acid are replaced with Alanine (A).

Embodiment 76. The pro-antibody of Embodiment 75, wherein the masking moiety comprises an amino acid sequence having at least 90%, 91%, 92%, 93%, 94%, 95%, 96%, 97%, 98%, or 99% percent identity to SEQ ID NO: 28.

Embodiment 77. The pro-antibody of any one of Embodiments 59 to 76, wherein the antigen-binding moiety comprises a heavy chain variable region (VH) comprising an amino acid sequence having at least 90%, 91%, 92%, 93%, 94%, 95%, 96%, 97%, 98%, or 99% percent identity to SEQ ID NO: 4 or SEQ ID NO: 15; and a light chain variable region (VL) comprising an amino acid sequence having at least 90%, 91%, 92%, 93%, 94%, 95%, 96%, 97%, 98%, or 99% percent identity to SEQ ID NO: 11.

Embodiment 78. The pro-antibody of Embodiment 77, wherein the 3^{rd} amino acid of the VH is Glutamine (Q), the 19^{th} amino acid of the VH is Arginine (R), the 20^{th} amino acid of the VH is Leucine (L), the 23^{rd} amino acid of the VH is Alanine (A), the 40^{th} amino acid of the VH is Alanine (A), the 84^{th} amino acid of the VH is Asparagine (N), the 85^{th} amino acid of the VH is Serine(S), the 87^{th} amino acid of the VH is Arginine (R), the 88^{th} amino acid of the VH is Alanine (A), and/or the 93^{rd} amino acid of the VH is Valine (V).

Embodiment 79. The pro-antibody of any one of Embodiments 59 to 78, wherein the 2^{nd} amino acid of the VL is Isoleucine (I), the 3^{rd} amino acid of the VL is Valine (V), the 7^{th} amino acid of the VL is Threonine (T), the 14^{th} amino acid of the VL is Threonine (T), the 17^{th} amino acid of the VL is Glutamine (Q), the 18^{th} amino acid of the VL is Proline (P), the 88th amino acid of the VL is Valine (V), the 105^{th} amino acid of the VL is Proline (P), the 109^{th} amino acid of the VL is Valine (V), and/or the 110^{th} amino acid of the VL is Aspartic acid (D).

Embodiment 80. The pro-antibody of any one of Embodiments 59 to 79, wherein the antigen-binding moiety comprises a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1, a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2, and a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3.

Embodiment 81. The pro-antibody of Embodiment 80, wherein the 4^{th} amino acid of the VH CDR 1 is Isoleucine (I), the 5^{th} amino acid of the VH CDR2 is Aspartic acid (D), the 12^{th} amino acid of the VH CDR2 is Glutamine (Q), the 15^{th} amino acid of the VH CDR2 is Leucine (L), the 4^{th} amino acid of the VH CDR3 is Serine(S), and/or the 5^{th} amino acid of the VH CDR3 is Glutamic acid (E).

Embodiment 82. The pro-antibody of any one of Embodiments 59 to 81, wherein the antigen-binding moiety comprises a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8, a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

Embodiment 83. The pro-antibody of Embodiment 82, wherein the 5^{th} amino acid of the VL CDR1 is Asparagine (N) and/or the 9^{th} amino acid of the VL CDR 1 is Threonine (T).

Embodiment 84. The pro-antibody of any one of Embodiments 59 to 83, wherein the antigen-binding moiety comprises a heavy chain comprising an amino acid sequence having at least 90%, 91%, 92%, 93%, 94%, 95%, 96%, 97%, 98%, or 99% percent identity to SEQ ID NO: 5 or SEQ ID NO: 16; and a light chain comprising an amino acid sequence having at least 90%, 91%, 92%, 93%, 94%, 95%, 96%, 97%, 98%, or 99% percent identity to SEQ ID NO: 12.

Embodiment 85. The pro-antibody of any one of Embodiments 59 to 84, wherein antigen-binding moiety has a binding specificity to Lewis Y antigen.

Embodiment 86. The pro-antibody of any one of Embodiments 59 to 85, wherein antigen-binding moiety does not bind Lewis X (LeX), Lewis A (LeA), Lewis B (LeB), or H type 2 antigen.

Embodiment 87. The pro-antibody of any one of Embodiments 59 to 86, wherein the proteinase substrate is cleavable by a matrix metalloproteinase (MMP), urokinase (Upa), matriptase, Factor Xa, legumain, tissue plasminogen activator (tPA), or ADAM metalloproteinases.

Embodiment 88. The pro-antibody of Embodiment 87, wherein the proteinase substrate comprises SEQ ID NO: 32, SEQ ID NO: 33, or SEQ ID NO: 34.

Embodiment 89. The pro-antibody of any one of Embodiments 59 to 88, wherein the linker comprises an amino acid sequence having at least 90%, 91%, 92%, 93%, 94%, 95%, 96%, 97%, 98%, or 99% percent identity to SEQ ID NO: 41, SEQ ID NO: 42, or SEQ ID NO: 43.

Embodiment 90. A pro-antibody, comprising a means for binding a Lewis Y antigen and a masking moiety, wherein the means for binding a Lewis Y antigen and the masking moiety are linked via a cleavable linker, comprising a proteinase substrate flanked by a first linkage moiety and a second linkage moiety, and at least one of the first linkage moiety and the second linkage moiety comprises an amino acid sequence of PLAQ (SEQ ID NO: 37), and further wherein the means for binding a Lewis Y antigen does not bind a Lewis X antigen.

Embodiment 91. The pro-antibody of Embodiment 90, wherein at least one of the first linkage moiety and the second linkage moiety comprises an amino acid sequence of GGS.

Embodiment 92. The pro-antibody of Embodiment 90 or Embodiment 91, wherein both the first linkage moiety and the second linkage moiety comprise the amino acid sequence of GGS.

Embodiment 93. The pro-antibody of any one of Embodiments 90 to 92, wherein the first linkage moiety connects the masking moiety to the proteinase substrate, and the second linkage moiety connects the proteinase substrate to the means for binding to Lewis Y antigen.

Embodiment 94. The pro-antibody of any one of Embodiments 90 to 93, wherein the first linkage moiety comprises an amino acid sequence of PLAQG (SEQ ID NO: 38), and the second linkage moiety comprises an amino acid sequence of GGGGS (SEQ ID NO: 39).

Embodiment 95. The pro-antibody of Embodiment 94, wherein the first linkage moiety comprises an amino acid sequence of GGSPLAQG (SEQ ID NO: 40).

Embodiment 96. The pro-antibody of any one of Embodiments 90 to 105, wherein the linker is a first linker and the proteinase substrate is a first proteinase substrate, and the pro-antibody further comprises a second linker, and further wherein the first linker connects the masking moiety to a heavy chain of the means for binding a Lewis Y antigen, and the second linker connects the masking moiety to a light chain of the means for binding a Lewis Y antigen; and the second linker comprises a second proteinase substrate flanked by a third linkage moiety and a fourth linkage moiety, and at least one of the third linkage moiety and the fourth linkage moiety comprises an amino acid sequence of PLAQ (SEQ ID NO: 37).

Embodiment 97. The pro-antibody of Embodiment 96, wherein at least one of the third linkage moiety and the fourth linkage moiety comprises an amino acid sequence of GGS

Embodiment 98. The pro-antibody of Embodiment 96 or Embodiment 97, wherein both the third linkage moiety and the fourth linkage moiety comprise the amino acid sequence of GGS.

Embodiment 99. The pro-antibody of any one of Embodiments 96 to 98, wherein the third linkage moiety comprises an amino acid sequence of PLAQG (SEQ ID NO: 38), and the fourth linkage moiety comprises an amino acid sequence of GGGGS (SEQ ID NO: 39).

Embodiment 100. The pro-antibody of Embodiment 99, wherein the third linkage moiety comprises an amino acid sequence of GGSPLAQG (SEQ ID NO: 40).

Embodiment 101. The pro-antibody of Embodiments 90 to 100, wherein the masking moiety comprises an amino acid sequence having at least 90%, 91%, 92%, 93%, 94%, 95%, 96%, 97%, 98%, or 99% percent identity to SEQ ID NO: 27.

Embodiment 102. The pro-antibody of Embodiment 101, wherein the masking moiety has at least one of Serine(S) or Threonine (T) thereof being replaced with an amino acid other than Serine(S) and Threonine (T).

Embodiment 103. The pro-antibody of Embodiment 102, wherein at least one of the 4^{th}, the 8^{th}, and the 10^{th} amino acids is replaced with an amino acid other than Serine(S) and Threonine (T).

Embodiment 104. The pro-antibody of Embodiment 102 or Embodiment 103, wherein the amino acid other than Serine(S) and Threonine (T) is Alanine (A) or Glycine (G).

Embodiment 105. The pro-antibody of any one of Embodiments 102 to 104, wherein only one of the 4^{th}, the 8^{th}, and the 10^{th} amino acids is replaced with the amino acid other than Serine(S) and Threonine (T), provided that when the 8^{th} amino acid of the linker is not Alanine (A), at least one of the 4^{th} and the 10^{th} amino acids is replaced with Alanine (A).

Embodiment 106. The pro-antibody of any one of Embodiments 102 to 105, wherein the 4^{th} amino acid, the 8^{th} amino acid, and the 10^{th} amino acid are replaced with Alanine (A).

Embodiment 107. The pro-antibody of Embodiment 106, wherein the masking moiety comprises an amino acid sequence having at least 90%, 91%, 92%, 93%, 94%, 95%, 96%, 97%, 98%, or 99% percent identity to the amino acid sequence set forth in SEQ ID NO: 28.

Embodiment 108. An isolated nucleic acid encoding the pro-antibody of any one of Embodiments 40 to 107.

Embodiment 109. A recombinant vector, comprising the isolated nucleic acid of Embodiment 108.

Embodiment 110. A host cell, comprising the recombinant vector of any one of Embodiments 31 to 39 and 109.

Embodiment 111. An antibody conjugate comprising a payload molecule covalently linked to the isolated antibody or the antigen-binding fragment thereof of any one of Embodiments 1 to 25 or the antigen-binding moiety of the pro-antibody of any one of Embodiments 40 to 107.

Embodiment 112. The antibody conjugate of Embodiment 111, wherein the antibody or the antigen-binding fragment thereof or the pro-antibody comprises a non-native cysteine or a non-native non-natural amino acid configured for site-specific conjugation.

Embodiment 113. The antibody conjugate of Embodiment 111 or Embodiment 112, wherein the payload molecule is covalently linked by a linker to the antibody or antigen-binding fragment thereof.

Embodiment 114. The antibody conjugate of any one of Embodiments 111 to 113, wherein the payload molecule is a therapeutic agent, a detectable indicator, or a combination thereof.

Embodiment 115. The antibody conjugate of any one of Embodiments 111 to 114, wherein the therapeutic agent is selected from a group consisting of mayntansinoid, auristatin, a PBD dimer, monomethylauristatin E (MMAE), monomethylauristatin F (MMAF), calicheamicin, 5-fluorouracil, methotrexate, doxorubicin, calicheamicins, pyrrolobenzodiazepines, duocarmycins, camptothecin, amantin, exatecan, 7-Ethyl-10-hydroxycamptothecin (SN38), deruxtecan (DXd), PNU-159682, DM1, DM4, and derivatives thereof.

Embodiment 116. A pharmaceutical composition, comprising the isolated antibody or the antigen-binding fragment thereof of any one of Embodiments 1 to 25, the pro-antibody of any one of Embodiments 40 to 107, and/or the antibody conjugate of any one of Embodiments 111 to 115; and a pharmaceutically acceptable excipient.

Embodiment 117. The pharmaceutical composition of Embodiment 116, wherein the pharmaceutically acceptable excipient comprises a binder, a diluent, a disintegrant, a filler, a glidant, a lubricant, a coloring agent, a preservative, a sweetener, a surfactant, a solvent, a coating agent, or a combination thereof.

Embodiment 118. A method of treating cancer, comprising administering the isolated antibody or the antigen-binding fragment thereof of any one of Embodiments 1 to 25, the pro-antibody of any one of Embodiments 40 to 107, the antibody conjugate of any one of Embodiments 111 to 115, or the pharmaceutical composition of Embodiment 116 or Embodiment 117 to a subject in need thereof, wherein the payload molecule is a therapeutic agent.

Embodiment 119. The method of Embodiment 118, wherein the cancer is a Lewis Y-expressing cancer.

Embodiment 120. The method of Embodiment 118 or Embodiment 119, wherein the cancer comprises bladder cancer, colon cancer, pancreas cancer, stomach cancer, lung cancer, esophagus cancer, cervix cancer, head and neck cancer, uterus cancer, breast cancer, or liver cancer.

Embodiment 121. The method of Embodiment 120, wherein the cancer is stomach cancer, bladder cancer, or lung cancer.

Embodiment 122. The method of any one of Embodiments 118 to 121, wherein the administering is performed via a route selected from a group consisting of intravenous, parenteral, intragastric, intrapleural, intrapulmonary, intrarectal, intradermal, intraperitoneal, intratumoral, subcutaneous, oral, topical, transdermal, intracisternal, intrathecal, intranasal, and intramuscular.

Embodiment 123. A method of detecting cancer in a subject in need thereof, comprising administering the isolated antibody or the antigen-binding fragment thereof of any one of Embodiments 1 to 25, the pro-antibody of any one of Embodiments 40 to 107, the antibody conjugate of any one of Embodiments 111 to 115 or the pharmaceutical composition of Embodiment 116 or Embodiment 117 to the subject in need thereof, wherein the payload molecule is a detectable indicator.

Embodiment 124. The method of Embodiment 123, wherein the cancer is a Lewis Y-expressing cancer.

Embodiment 125. The method of Embodiment 123 or Embodiment 124, wherein the cancer comprises bladder cancer, colon cancer, pancreas cancer, stomach cancer, lung cancer, esophagus cancer, cervix cancer, head and neck cancer, uterus cancer, breast cancer, or liver cancer.

Embodiment 126. The method of Embodiment 125, wherein the cancer is stomach cancer, bladder cancer, or lung cancer.

Embodiment 127. The method of any one of Embodiments 123 to 126, wherein the administering is conducted via a route selected from a group consisting of intravenous, parenteral, intragastric, intrapleural, intrapulmonary, intrarectal, intradermal, intraperitoneal, intratumoral, subcutaneous, oral, topical, transdermal, intracisternal, intrathecal, intranasal, and intramuscular.

Embodiment 128. The method of any one of Embodiments 123 to 127, wherein the detectable indicator is a radionuclide selected from a group consisting of ⁶⁸Ga, ⁶⁴Cu, ⁸⁶Y, ⁸⁹Zr, ¹²⁴I, ^{99m}Tc, ²¹³I, ¹¹¹In, ¹⁷⁷Lu, ¹³¹I, ⁷⁶Br, ⁷⁸Zr, ¹⁸F, and ¹²⁴T.

SEQUENCE LISTING

| | |
|---|---|
| SEQ ID NO: 1 | DYYIY |
| HMK4 VHCDR1 (aa) | |
| Artificial | |
| sequence | |
| SEQ ID NO: 2 | YISQDGDITD YQDTLKG |
| HMK4 VHCDR2 (aa) | |
| Artificial | |
| sequence | |
| SEQ ID NO: 3 | GLSEGAWFAY |

Artificial

| | |
|---|---|
| HMK4 VHCDR3 (aa) | |
| Artificial | |
| sequence | |
| SEQ ID NO: 4 | EVQLVESGGG LVQPGGSLRL SCATSGFTFS DYYIYWVRQA |
| HKM4 VH (aa) | PEKRLEWVAY ISQDGDITDY QDTLKGRFTI SRDNAKNTLY |
| Artificial | LQMNSLRAED TAVYYCARGL SEGAWFAYWG QGTLVTVSS |
| sequence | |
| SEQ ID NO: 5 | EVQLVESGGG LVQPGGSLRL SCATSGFTFS DYYIYWVRQA |
| HKM4 heavy chain | PEKRLEWVAY ISQDGDITDY QDTLKGRFTI SRDNAKNTLY |
| (aa) Artificial | LQMNSLRAED TAVYYCARGL SEGAWFAYWG QGTLVTVSSA |
| sequence | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCDKTHTCPP CPAPELLGGP |
| | SVFLFPPKPK DTLMISRTPE VTCVVVDVSH EDPEVKENWY |
| | VDGVEVHNAK TKPREEQYNS TYRVVSVLTV LHQDWLNGKE |
| | YKCKVSNKAL PAPIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| SEQ ID NO: 6 | gaggtgcagc tggttgaatc tggcggagga ttggttcagc |
| HKM4 VH (nt) | ctggcggctc tctgagactg tcttgtgcca cctctggctt |
| Artificial | caccttctcc gactactaca tctactgggt ccgacaggcc |
| sequence | cctgagaaac ggcttgagtg ggtggcctac atcagccagg |
| | acggcgacat caccgactac caggataccc tgaagggcag |
| | attcaccatc agccgggaca acgccaagaa caccctgtac |
| | ctgcagatga actccctgag agccgaggac accgccgtgt |
| | actattgtgc tagaggcctg tccgaaggcg cttggtttgc |
| | ttattggggc cagggcacac tggtcaccgt gtcctct |
| SEQ ID NO: 7 | Gaggtgcagc tggttgaatc tggcggagga ttggttcagc |
| HKM4 heavy chain | ctggcggctc tctgagactg tcttgtgcca cctctggctt |
| (nt) Artificial | caccttctcc gactactaca tctactgggt ccgacaggcc |
| sequence | cctgagaaac ggcttgagtg ggtggcctac atcagccagg |
| | acggcgacat caccgactac caggataccc tgaagggcag |
| | attcaccatc agccgggaca acgccaagaa caccctgtac |
| | ctgcagatga actccctgag agccgaggac accgccgtgt |
| | actattgtgc tagaggcctg tccgaaggcg cttggtttgc |
| | ttattggggc cagggcacac tggtcaccgt gtcctctgct |
| | tctacaaagg gcccatcggt cttccccctg gcaccctcct |
| | ccaagagcac ctctgggggc acagcggccc tgggctgcct |
| | ggtcaaggac tacttccccg aaccggtgac ggtgtcgtgg |
| | aactcaggcg ccctgaccag cggcgtgcac accttcccgg |
| | ctgtcctaca gtcctcagga ctctactccc tcagcagcgt |
| | ggtgaccgtg ccctccagca gcttgggcac ccagacctac |
| | atctgcaacg tgaatcacaa gcccagcaac accaaggtgg |
| | acaagaaagt tgagcccaaa tottgtgaca aaactcacac |
| | atgcccaccg tgcccagcac ctgaactcct ggggggaccg |
| | tcagtcttcc tcttcccccc aaaacccaag gacaccctca |
| | tgatctcccg gacccctgag gtcacatgcg tggtggtgga |
| | cgtgagccac gaagaccctg aggtcaagtt caactggtac |
| | gtggacggcg tggaggtgca taatgccaag acaaagccgc |
| | gggaggagca gtacaacagc acgtaccgtg tggtcagcgt |
| | cctcaccgtc ctgcaccagg actggctgaa tggcaaggag |
| | tacaagtgca aggtctccaa caaagccctc ccagccccca |
| | tcgagaaaac catctccaaa gccaaagggc agccccgaga |
| | accacaggtg tacaccctgc ccccatcccg ggatgagctg |
| | accaagaacc aggtcagcct gacctgcctg gtcaaaggct |
| | tctatcccag cgacatcgcc gtggagtggg agagcaatgg |
| | gcagccggag aacaactaca agaccacgcc tcccgtgctg |
| | gactccgacg gctccttctt cctctacagc aagctcaccg |
| | tggacaagag caggtggcag caggggaacg tcttctcatg |
| | ctccgtgatg catgaggctc tgcacaacca ctacacgcag |
| | aagagcctct ccctgtctcc gggtaaatga |
| SEQ ID NO: 8 | RSSQNIVHTN GNTYLE |
| HMK4 VLCDR1 (aa) | |
| Artificial | |
| sequence | |
| SEQ ID NO: 9 | KVSNRFS |
| HMK4 VLCDR2 (aa) | |
| Artificial | |
| sequence | |
| SEQ ID NO: 10 | FQGSHVPFT |
| HMK4 VLCDR3 (aa) | |
| Artificial | |
| sequence | |
| SEQ ID NO: 11 | DIVMTQTPLS LPVTLGQPAS ISCRSSQNIV HTNGNTYLEW |
| HKM4 VL (aa) | YLQKPGQSPQ LLIYKVSNRF SGVPDRESGS GSGTDFTLKI |
| Artificial | SRVEAEDVGV YYCFQGSHVP FTFGPGTKVD IK |
| sequence | |
| SEQ ID NO: 12 | DIVMTQTPLS LPVTLGQPAS ISCRSSQNIV HTNGNTYLEW |
| HKM4 light chain | YLQKPGQSPQ LLIYKVSNRF SGVPDRESGS GSGTDFTLKI |
| (aa) Artificial | SRVEAEDVGV YYCFQGSHVP FTFGPGTKVD IKRTVAAPSV |
| sequence | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSENRGEC |
| SEQ ID NO: 13 | gacattgtga tgacccagac acctctgagc ctgcccgtga |
| HMK4 VL (nt) | cactgggaca gcctgcctcc atctcctgca gatccagcca |
| Artificial | gaacatcgtg cacaccaacg gcaacaccta cctggaatgg |
| sequence | tatctgcaga agcccggcca gtctcctcag ctgctgatct |
| | acaaggtgtc caaccggttc tctggcgtgc ccgacagatt |
| | ttccggctct ggctctggca ccgacttcac cctgaagatt |
| | tccagagtgg aagccgagga tgtgggcgtg tactactgct |
| | tccaaggctc tcacgtgcca ttcaccttcg gacctggcac |
| | caaggtggac atcaag |
| SEQ ID NO: 14 | Gacattgtga tgacccagac acctctgagc ctgcccgtga |
| HKM4 light chain | cactgggaca gcctgcctcc atctcctgca gatccagcca |
| (nt) Artificial | gaacatcgtg cacaccaacg gcaacaccta cctggaatgg |
| sequence | tatctgcaga agcccggcca gtctcctcag ctgctgatct |
| | acaaggtgtc caaccggttc tctggcgtgc ccgacagatt |
| | ttccggctct ggctctggca ccgacttcac cctgaagatt |
| | tccagagtgg aagccgagga tgtgggcgtg tactactgct |
| | tccaaggctc tcacgtgcca ttcaccttcg gacctggcac |
| | caaggtggac atcaagcgta cggtggctgc accatctgtc |
| | ttcatcttcc cgccatctga tgagcagttg aaatctggaa |
| | ctgcctctgt tgtgtgcctg ctgaataact tctatcccag |
| | agaggccaaa gtacagtgga aggtggataa cgccctccaa |
| | tcgggtaact cccaggagag tgtcacagag caggacagca |
| | aggacagcac ctacagcctc agcagcaccc tgacgctgag |
| | caaagcagac tacgagaaac acaaagtcta cgcctgcgaa |
| | gtcacccatc agggcctgag ctcgcccgtc acaaagagct |
| | tcaacagggg agagtgttaa |
| SEQ ID NO: 15 | EVQLVESGGG LVQPGGSLRL SCATSGFTES DYYIYWVRQA |
| HKM1 VH (aa) | PGKGLEWVAY ISQDGDITDY QDTLKGRFTI SRDNAKNTLY |
| Artificial | LQMNSLRAED TAVYYCARGL SEGAWFAYWG QGTLVTVSS |
| sequence | |
| SEQ ID NO: 16 | EVQLVESGGG LVQPGGSLRL SCATSGFTFS DYYIYWVRQA |
| HKM1 heavy chain | PGKGLEWVAY ISQDGDITDY QDTLKGRFTI SRDNAKNTLY |
| (aa) | LQMNSLRAED TAVYYCARGL SEGAWFAYWG QGTLVTVSSA |
| Artificial | STKGPSVFPL APSSKSTSGG TAALGCLVKD YFPEPVTVSW |
| sequence | NSGALTSGVH TFPAVLQSSG LYSLSSVVTV PSSSLGTQTY |
| | ICNVNHKPSN TKVDKKVEPK SCDKTHTCPP CPAPELLGGP |
| | SVFLFPPKPK DTLMISRTPE VTCVVVDVSH EDPEVKENWY |
| | VDGVEVHNAK TKPREEQYNS TYRVVSVLTV LHQDWLNGKE |
| | YKCKVSNKAL PAPIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| SEQ ID NO: 17 | DYYMY |
| BR96 VHCDR1 (aa) | |
| Artificial | |
| sequence | |
| SEQ ID NO: 18 | YISQGGDITD YPDTVKG |
| BR96 VHCDR2 (aa) | |
| Artificial | |
| sequence | |
| SEQ ID NO: 19 | GLDDGAWFAY |
| BR96 VHCDR3 (aa) | |
| Artificial | |
| sequence | |
| SEQ ID NO: 20 | EVNLVESGGG LVQPGGSLKV SCVTSGFTFS DYYMYWVRQT |
| BR96 VH (aa) | PEKRLEWVAY ISQGGDITDY PDTVKGRFTI SRDNAKNTLY |
| Artificial | LQMSRLKSED TAMYYCARGL DDGAWFAYWG QGTLVTVSV |
| sequence | |
| SEQ ID NO: 21 | EVNLVESGGG LVQPGGSLKV SCVTSGFTFS DYYMYWVRQT |
| BR96 heavy chain | PEKRLEWVAY ISQGGDITDY PDTVKGRFTI SRDNAKNTLY |
| (aa) Artificial | LQMSRLKSED TAMYYCARGL DDGAWFAYWG QGTLVTVSVA |
| sequence | STKGPSVFPL APSSKSTSGG TAALGCLVKD YFPEPVTVSW |
| | NSGALTSGVH TFPAVLQSSG LYSLSSVVTV PSSSLGTQTY |
| | ICNVNHKPSN TKVDKKVEPK SCDKTHTCPP CPAPELLGGP |
| | SVFLFPPKPK DTLMISRTPE VTCVVVDVSH EDPEVKENWY |
| | VDGVEVHNAK TKPREEQYNS TYRVVSVLTV LHQDWLNGKE |
| | YKCKVSNKAL PAPIEKTISK AKGQPREPQV YTLPPSRDEL |
| | TKNQVSLTCL VKGFYPSDIA VEWESNGQPE NNYKTTPPVL |
| | DSDGSFFLYS KLTVDKSRWQ QGNVFSCSVM HEALHNHYTQ |
| | KSLSLSPGK |
| SEQ ID NO: 22 | RSSQIIVHNN GNTYLE |
| BR96 VLCDR1 (aa) | |
| Artificial | |
| sequence | |
| SEQ ID NO: 23 | KVSNRFS |
| BR96 VLCDR2 (aa) | |
| Artificial | |
| sequence | |
| SEQ ID NO: 24 | FQGSHVPFT |
| BR96 VLCDR3 (aa) | |
| Artificial | |
| sequence | |
| SEQ ID NO: 25 | DVLMTQIPVS LPVSLGDQAS ISCRSSQIIV HNNGNTYLEW |
| BR96 VL (aa) | YLQKPGQSPQ LLIYKVSNRF SGVPDRESGS GSGTDFTLKI |
| Artificial | SRVEAEDLGV YYCFQGSHVP FTFGSGTKLE IK |
| sequence | |
| SEQ ID NO: 26 | DVLMTQIPVS LPVSLGDQAS ISCRSSQIIV HNNGNTYLEW |
| BR96 light chain | YLQKPGQSPQ LLIYKVSNRF SGVPDRESGS GSGTDFTLKI |
| (aa) Artificial | SRVEAEDLGV YYCFQGSHVP FTFGSGTKLE IKRTVAAPSV |
| sequence | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSENRGEC |
| SEQ ID NO: 27 | EPKSCDKTHT CPPCP |
| Masking moiety- | |
| L1 (aa) | |
| Artificial | |
| sequence | |
| SEQ ID NO: 28 | EPKACDKAHA CPPCP |
| Masking moiety- | |
| L6 (aa) | |
| Artificial | |
| sequence | |
| SEQ ID NO: 29 | GGSGPLGVRG GGGS |
| Linker (aa) | |
| Artificial | |
| sequence | |
| SEQ ID NO: 30 | GGSIPVSLRS GGGGGS |
| Linker (aa) | |
| Artificial | |
| sequence | |
| SEQ ID NO: 31 | GGSPLGLAGG GGGS |
| Linker (aa) | |
| Artificial | |
| sequence | |
| SEQ ID NO: 32 | GPLGVR |
| Protease | |
| substrate S1 (aa) | |
| Artificial | |
| sequence | |
| SEQ ID NO: 33 | IPVSLRSG |
| Protease | |
| substrate S2 (aa) | |
| Artificial | |
| sequence | |
| SEQ ID NO: 34 | PLGLAG |
| Protease | |
| substrate S3 (aa) | |
| Artificial | |
| sequence | |
| SEQ ID NO: 35 | EPKSCDKAHT CPPCP |
| Masking moiety- | |
| L5 (aa) | |
| Artificial | |
| sequence | |
| SEQ ID NO: 36 | THTCPPCP |
| Masking Moiety | |
| (aa) Artificial | |
| sequence | |
| SEQ ID NO: 37 | PLAQ |
| Linkage moiety | |
| (aa) Artificial | |
| sequence | |
| SEQ ID NO: 38 | PLAQG |
| Linkage moiety | |
| (aa) Artificial | |
| sequence | |
| SEQ ID NO: 39 | GGGGS |
| Linkage moiety | |
| (aa) Artificial | |
| sequence | |
| SEQ ID NO: 40 | GGSPLAQG |
| Linkage moiety | |
| (aa) Artificial | |
| sequence | |
| SEQ ID NO: 41 | GGSPLAQGGP LGVRGGGGS |
| Linker (aa) | |
| Artificial | |
| sequence | |
| SEQ ID NO: 42 | GGSPLAQGIP VSLRSGGGGG S |
| Linker (aa) | |
| Artificial | |
| sequence | |
| SEQ ID NO: 43 | GGSPLAQGP LGLAGGGGGS |
| Linker (aa) | |
| Artificial | |
| sequence | |
| SEQ ID NO: 44 | EVQLVESGGG LVQPGGSLRL SCATSGFTFS DYYIYWVRQA |
| ScFV-HKM4 (aa) | PEKRLEWVAY ISQDGDITDY QDTLKGRFTI SRDNAKNTLY |
| Artificial | LQMNSLRAED TAVYYCARGL SEGAWFAYWG QGTLVTVSS (GGGGS)ₓ |
| sequence | DIVMTQTPLS LPVTLGQPAS ISCRSSQNIV HTNGNTYLEW |
| | YLQKPGQSPQ LLIYKVSNRF SGVPDRESGS GSGTDFTLKI |
| | SRVEAEDVGV YYCFQGSHVP FTFGPGTKVD IKRTVAAPSV |
| | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSENRGEC |
| | wherein “x” can be 1, or 2, 3, 4, 5, 6, 7, 8, 9, |
| | 10, or more repeats of the sequence GGGGS shown in |
| | parentheses. |
| SEQ ID NO: 45 | EVQLVESGGG LVQPGGSLRL SCATSGFTFS DYYIYWVRQA |
| ScFV-HKM1 (aa) | PGKGLEWVAY ISQDGDITDY QDTLKGRFTI SRDNAKNTLY |
| Artificial | LQMNSLRAED TAVYYCARGL SEGAWFAYWG QGTLVTVSS (GGGGS)ₓ |
| sequence | DIVMTQTPLS LPVTLGQPAS ISCRSSQNIV HTNGNTYLEW |
| | YLQKPGQSPQ LLIYKVSNRF SGVPDRFSGS GSGTDFTLKI |
| | SRVEAEDVGV YYCFQGSHVP FTFGPGTKVD IKRTVAAPSV |
| | FIFPPSDEQL KSGTASVVCL LNNFYPREAK VQWKVDNALQ |
| | SGNSQESVTE QDSKDSTYSL SSTLTLSKAD YEKHKVYACE |
| | VTHQGLSSPV TKSENRGEC |
| | wherein “x” can be 1, or 2, 3, 4, 5, 6, 7, 8, 9, |
| | 10, or more repeats of the sequence GGGGS shown in |
| | parentheses. |
| SEQ ID NO: 46 | gaggtgcagc tggttgaatc tggcggagga ttggttcagc |
| HKM1 VH (nt) | ctggcggctc tctgagactg tcttgtgcca cctctggctt |
| Artificial | caccttctcc gactactaca tctactgggt ccgacaggcc |
| sequence | cctggcaaag gacttgagtg ggtggcctac atcagccagg |
| | acggcgacat caccgactac caggataccc tgaagggcag |
| | attcaccatc agccgggaca acgccaagaa caccctgtac |
| | ctgcagatga actccctgag agccgaggac accgccgtgt |
| | actattgtgc tagaggcctg tccgaaggcg cttggtttgc |
| | ttattggggc cagggcacac tggtcaccgt gtcctct |

1. A pro-antibody, comprising an antigen-binding moiety and a masking moiety,
wherein the antigen-binding moiety and the masking moiety are linked via a cleavable linker, comprising a proteinase substrate flanked by a first linkage moiety and a second linkage moiety, and at least one of the first linkage moiety and the second linkage moiety comprises an amino acid sequence of PLAQ (SEQ ID NO: 37); and
wherein the antigen-binding moiety comprises a heavy chain variable region (VH) comprising an amino acid sequence having at least 90%, 91%, 92%, 93%, 94%, 95%, 96%, 97%, 98%, or 99% percent identity to SEQ ID NO: 4; and a light chain variable region (VL) comprising an amino acid sequence having at least 90%, 91%, 92%, 93%, 94%, 95%, 96%, 97%, 98%, or 99% percent identity to SEQ ID NO: 11.

2. The pro-antibody of claim 1, wherein at least one of the first linkage moiety and the second linkage moiety comprises an amino acid sequence of Gly-Gly-Ser (GGS).

3. The pro-antibody of claim 1, wherein the first linkage moiety connects the masking moiety to the proteinase substrate, and the second linkage moiety connects the proteinase substrate to the antigen-binding moiety.

4. The pro-antibody of claim 1, wherein the first linkage moiety comprises an amino acid sequence of PLAQG (SEQ ID NO: 38), and the second linkage moiety comprises an amino acid sequence of GGGGS (SEQ ID NO: 39).

5. The pro-antibody of claim 4, wherein the first linkage moiety comprises an amino acid sequence of GGSPLAQG (SEQ ID NO: 40).

6. The pro-antibody of claim 1, wherein the linker is a first linker and the proteinase substrate is a first proteinase substrate, and the pro-antibody further comprises a second linker, and further wherein
the first linker connects the masking moiety to a heavy chain of the antigen-binding moiety, and the second linker connects the masking moiety to a light chain of the antigen-binding moiety; and
the second linker comprises a second proteinase substrate flanked by a third linkage moiety and a fourth linkage moiety, and at least one of the third linkage moiety and the fourth linkage moiety comprises an amino acid sequence of PLAQ (SEQ ID NO: 37).

7. The pro-antibody of claim 6, wherein the third linkage moiety comprises an amino acid sequence of PLAQG (SEQ ID NO: 38), and the fourth linkage moiety comprises an amino acid sequence of GGGGS (SEQ ID NO: 39).

8. The pro-antibody of claim 7, wherein the third linkage moiety comprises an amino acid sequence of GGSPLAQG (SEQ ID NO: 40).

9. The pro-antibody of claim 1, wherein the masking moiety comprises an amino acid sequence having at least 90%, 91%, 92%, 93%, 94%, 95%, 96%, 97%, 98%, or 99% percent identity to SEQ ID NO: 27.

10. The pro-antibody of claim 9, wherein at least one of the 4^{th}, the 8^{th}, and the 10^{th} amino acids is replaced with the amino acid other than Serine(S) and Threonine (T).

11. The pro-antibody of claim 10, wherein only one of the 4^{th}, the 8^{th}, and the 10^{th} amino acids is replaced with the amino acid other than Serine(S) and Threonine (T), provided that when the 8^{th} amino acid of the linker is not Alanine (A), at least one of the 4^{th} and the 10^{th} amino acids is replaced with the amino acid other than Serine(S) and Threonine (T).

12. The pro-antibody of claim 10, wherein the 4^{th} amino acid, the 8^{th} amino acid, and the 10th amino acid are replaced with Alanine (A).

13. The pro-antibody of claim 1, wherein the 3^{rd} amino acid of the VH is Glutamine (Q), the 19^{th} amino acid of the VH is Arginine (R), the 20^{th} amino acid of the VH is Leucine (L), the 23^{rd} amino acid of the VH is Alanine (A), the 40^{th} amino acid of the VH is Alanine (A), the 84^{th} amino acid of the VH is Asparagine (N), the 85^{th} amino acid of the VH is Serine(S), the 87^{th} amino acid of the VH is Arginine (R), the 88^{th} amino acid of the VH is Alanine (A), and/or the 93^{rd} amino acid of the VH is Valine (V); and/or
wherein the 2^{nd} amino acid of the VL is Isoleucine (I), the 3^{rd} amino acid of the VL is Valine (V), the 7^{th} amino acid of the VL is Threonine (T), the 14^{th} amino acid of the VL is Threonine (T), the 17^{th} amino acid of the VL is Glutamine (Q), the 18th amino acid of the VL is Proline (P), the 88^{th} amino acid of the VL is Valine (V), the 105^{th} amino acid of the VL is Proline (P), the 109^{th} amino acid of the VL is Valine (V), and/or the 110^{th} amino acid of the VL is Aspartic acid (D).

14. The pro-antibody of claim 1, wherein the antigen-binding moiety comprises a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1,
a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2,
a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3;
a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8,
a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and
a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

15. The pro-antibody of claim 14, wherein the 4^{th} amino acid of the VH CDR1 is Isoleucine (I), the 5^{th} amino acid of the VH CDR2 is Aspartic acid (D), the 12^{th} amino acid of the VH CDR2 is Glutamine (Q), the 15^{th} amino acid of the VH CDR2 is Leucine (L), the 4th amino acid of the VH CDR3 is Serine(S), the 5^{th} amino acid of the VH CDR3 is Glutamic acid (E), the 5^{th} amino acid of the VL CDR1 is Asparagine (N), and/or the 9th amino acid of the VL CDR1 is Threonine (T).

16. The pro-antibody of claim 1, wherein the antigen-binding moiety has a binding specificity to Lewis Y antigen, and the antigen-binding moiety does not bind Lewis X (LeX), Lewis A (LeA), Lewis B (LeB), or H type 2 antigen.

17. An isolated nucleic acid encoding the pro-antibody of claim 1.

18. A recombinant vector, comprising the isolated nucleic acid of claim 17.

19. A host cell, comprising the recombinant vector of claim 18.

20. An isolated antibody or an antigen-binding fragment thereof, comprising:
(a) a heavy chain variable region (VH) comprising
a heavy chain complementarity determining region 1 (VH CDR1) comprising an amino acid sequence that has at least 90%, 91%, 92%, 93%, 94%, 95%, 96%, 97%, 98%, or 99% percent identity to SEQ ID NO: 1,
a heavy chain complementarity determining region 2 (VH CDR2) comprising an amino acid sequence that has at least 90%, 91%, 92%, 93%, 94%, 95%, 96%, 97%, 98%, or 99% percent identity to SEQ ID NO: 2, and
a heavy chain complementarity determining region 3 (VH CDR3) comprising an amino acid sequence that has at least 90%, 91%, 92%, 93%, 94%, 95%, 96%, 97%, 98%, or 99% percent identity to SEQ ID NO: 3; and
(b) a light chain variable region (VL) comprising
a light chain complementarity determining region 1 (VL CDR1) comprising an amino acid sequence that has at least 90%, 91%, 92%, 93%, 94%, 95%, 96%, 97%, 98%, or 99% percent identity to SEQ ID NO: 8,
a light chain complementarity determining region 2 (VL CDR2) comprising an amino acid sequence that has at least 90%, 91%, 92%, 93%, 94%, 95%, 96%, 97%, 98%, or 99% percent identity to SEQ ID NO: 9, and
a light chain complementarity determining region 3 (VL CDR3) comprising an amino acid sequence that has at least 90%, 91%, 92%, 93%, 94%, 95%, 96%, 97%, 98%, or 99% percent identity to SEQ ID NO: 10.

21. An antibody conjugate comprising a payload molecule covalently linked to the antigen-binding moiety of the pro-antibody of claim 1 and the isolated antibody or the antigen-binding fragment thereof claim 20.

22. A pharmaceutical composition, comprising
the pro-antibody of claim 1, the isolated antibody or the antigen-binding fragment thereof of claim 20, and/or the antibody conjugate of claim 21; and
a pharmaceutically acceptable excipient.

23. A method of treating cancer, comprising administering the pro-antibody of claim 1, the isolated antibody or the antigen-binding fragment thereof of claim 20, the antibody conjugate of claim 21, or the pharmaceutical composition of claim 22 to a subject in need thereof, wherein the payload molecule is a therapeutic agent.


SEQUENCE LISTING (46 sequences)

SEQ ID NO: 1 - protein, 5 aa, synthetic construct
DYYIY

SEQ ID NO: 2 - protein, 17 aa, synthetic construct
YISQDGDITDYQDTLKG

SEQ ID NO: 3 - protein, 10 aa, synthetic construct
GLSEGAWFAY

SEQ ID NO: 4 - protein, 119 aa, synthetic construct
EVQLVESGGGLVQPGGSLRLSCATSGFTFSDYYIYWVRQAPEKRLEWVAYISQDGDITDYQDTLKGRFTISRDNAKNTLYLQMNSLRAEDTAVYYCARGLSEGAWFAYWGQGTLVTVSS

SEQ ID NO: 5 - protein, 449 aa, synthetic construct
EVQLVESGGGLVQPGGSLRLSCATSGFTFSDYYIYWVRQAPEKRLEWVAYISQDGDITDYQDTLKGRFTISRDNAKNTLYLQMNSLRAEDTAVYYCARGLSEGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 357 bp, synthetic construct
  source 1..357: mol_type = other DNA
gaggtgcagctggttgaatctggcggaggattggttcagcctggcggctctctgagactgtcttgtgccacctctggcttcaccttctccgactactacatctactgggtccgacaggcccctgagaaacggcttgagtgggtggcctacatcagccaggacggcgacatcaccgactaccaggataccctgaagggcagattcaccatcagccgggacaacgccaagaacaccctgtacctgcagatgaactccctgagagccgaggacaccgccgtgtactattgtgctagaggcctgtccgaaggcgcttggtttgcttattggggccagggcacactggtcaccgtgtcctct

SEQ ID NO: 7 - DNA, 1350 bp, synthetic construct
  source 1..1350: mol_type = other DNA
gaggtgcagctggttgaatctggcggaggattggttcagcctggcggctctctgagactgtcttgtgccacctctggcttcaccttctccgactactacatctactgggtccgacaggcccctgagaaacggcttgagtgggtggcctacatcagccaggacggcgacatcaccgactaccaggataccctgaagggcagattcaccatcagccgggacaacgccaagaacaccctgtacctgcagatgaactccctgagagccgaggacaccgccgtgtactattgtgctagaggcctgtccgaaggcgcttggtttgcttattggggccagggcacactggtcaccgtgtcctctgcttctacaaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 8 - protein, 16 aa, synthetic construct
RSSQNIVHTNGNTYLE

SEQ ID NO: 9 - protein, 7 aa, synthetic construct
KVSNRFS

SEQ ID NO: 10 - protein, 9 aa, synthetic construct
FQGSHVPFT

SEQ ID NO: 11 - protein, 112 aa, synthetic construct
DIVMTQTPLSLPVTLGQPASISCRSSQNIVHTNGNTYLEWYLQKPGQSPQLLIYKVSNRFSGVPDRFSGSGSGTDFTLKISRVEAEDVGVYYCFQGSHVPFTFGPGTKVDIK

SEQ ID NO: 12 - protein, 219 aa, synthetic construct
DIVMTQTPLSLPVTLGQPASISCRSSQNIVHTNGNTYLEWYLQKPGQSPQLLIYKVSNRFSGVPDRFSGSGSGTDFTLKISRVEAEDVGVYYCFQGSHVPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 336 bp, synthetic construct
  source 1..336: mol_type = other DNA
gacattgtgatgacccagacacctctgagcctgcccgtgacactgggacagcctgcctccatctcctgcagatccagccagaacatcgtgcacaccaacggcaacacctacctggaatggtatctgcagaagcccggccagtctcctcagctgctgatctacaaggtgtccaaccggttctctggcgtgcccgacagattttccggctctggctctggcaccgacttcaccctgaagatttccagagtggaagccgaggatgtgggcgtgtactactgcttccaaggctctcacgtgccattcaccttcggacctggcaccaaggtggacatcaag

SEQ ID NO: 14 - DNA, 660 bp, synthetic construct
  source 1..660: mol_type = other DNA
gacattgtgatgacccagacacctctgagcctgcccgtgacactgggacagcctgcctccatctcctgcagatccagccagaacatcgtgcacaccaacggcaacacctacctggaatggtatctgcagaagcccggccagtctcctcagctgctgatctacaaggtgtccaaccggttctctggcgtgcccgacagattttccggctctggctctggcaccgacttcaccctgaagatttccagagtggaagccgaggatgtgggcgtgtactactgcttccaaggctctcacgtgccattcaccttcggacctggcaccaaggtggacatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 15 - protein, 119 aa, synthetic construct
EVQLVESGGGLVQPGGSLRLSCATSGFTFSDYYIYWVRQAPGKGLEWVAYISQDGDITDYQDTLKGRFTISRDNAKNTLYLQMNSLRAEDTAVYYCARGLSEGAWFAYWGQGTLVTVSS

SEQ ID NO: 16 - protein, 449 aa, synthetic construct
EVQLVESGGGLVQPGGSLRLSCATSGFTFSDYYIYWVRQAPGKGLEWVAYISQDGDITDYQDTLKGRFTISRDNAKNTLYLQMNSLRAEDTAVYYCARGLSEGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 5 aa, synthetic construct
DYYMY

SEQ ID NO: 18 - protein, 17 aa, synthetic construct
YISQGGDITDYPDTVKG

SEQ ID NO: 19 - protein, 10 aa, synthetic construct
GLDDGAWFAY

SEQ ID NO: 20 - protein, 119 aa, synthetic construct
EVNLVESGGGLVQPGGSLKVSCVTSGFTFSDYYMYWVRQTPEKRLEWVAYISQGGDITDYPDTVKGRFTISRDNAKNTLYLQMSRLKSEDTAMYYCARGLDDGAWFAYWGQGTLVTVSV

SEQ ID NO: 21 - protein, 449 aa, synthetic construct
EVNLVESGGGLVQPGGSLKVSCVTSGFTFSDYYMYWVRQTPEKRLEWVAYISQGGDITDYPDTVKGRFTISRDNAKNTLYLQMSRLKSEDTAMYYCARGLDDGAWFAYWGQGTLVTVSVASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 16 aa, synthetic construct
RSSQIIVHNNGNTYLE

SEQ ID NO: 23 - protein, 7 aa, synthetic construct
KVSNRFS

SEQ ID NO: 24 - protein, 9 aa, synthetic construct
FQGSHVPFT

SEQ ID NO: 25 - protein, 112 aa, synthetic construct
DVLMTQIPVSLPVSLGDQASISCRSSQIIVHNNGNTYLEWYLQKPGQSPQLLIYKVSNRFSGVPDRFSGSGSGTDFTLKISRVEAEDLGVYYCFQGSHVPFTFGSGTKLEIK

SEQ ID NO: 26 - protein, 219 aa, synthetic construct
DVLMTQIPVSLPVSLGDQASISCRSSQIIVHNNGNTYLEWYLQKPGQSPQLLIYKVSNRFSGVPDRFSGSGSGTDFTLKISRVEAEDLGVYYCFQGSHVPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 15 aa, synthetic construct
EPKSCDKTHTCPPCP

SEQ ID NO: 28 - protein, 15 aa, synthetic construct
EPKACDKAHACPPCP

SEQ ID NO: 29 - protein, 14 aa, synthetic construct
GGSGPLGVRGGGGS

SEQ ID NO: 30 - protein, 16 aa, synthetic construct
GGSIPVSLRSGGGGGS

SEQ ID NO: 31 - protein, 14 aa, synthetic construct
GGSPLGLAGGGGGS

SEQ ID NO: 32 - protein, 6 aa, synthetic construct
GPLGVR

SEQ ID NO: 33 - protein, 8 aa, synthetic construct
IPVSLRSG

SEQ ID NO: 34 - protein, 6 aa, synthetic construct
PLGLAG

SEQ ID NO: 35 - protein, 15 aa, synthetic construct
EPKSCDKAHTCPPCP

SEQ ID NO: 36 - protein, 8 aa, synthetic construct
THTCPPCP

SEQ ID NO: 37 - protein, 4 aa, synthetic construct
PLAQ

SEQ ID NO: 38 - protein, 5 aa, synthetic construct
PLAQG

SEQ ID NO: 39 - protein, 5 aa, synthetic construct
GGGGS

SEQ ID NO: 40 - protein, 8 aa, synthetic construct
GGSPLAQG

SEQ ID NO: 41 - protein, 19 aa, synthetic construct
GGSPLAQGGPLGVRGGGGS

SEQ ID NO: 42 - protein, 21 aa, synthetic construct
GGSPLAQGIPVSLRSGGGGGS

SEQ ID NO: 43 - protein, 19 aa, synthetic construct
GGSPLAQGPLGLAGGGGGS

SEQ ID NO: 44 - protein, 343 aa, synthetic construct
  REPEAT 120..124: note = 1, or 2, 3, 4, 5, 6, 7, 8, 9, 10, or more repeats
EVQLVESGGGLVQPGGSLRLSCATSGFTFSDYYIYWVRQAPEKRLEWVAYISQDGDITDYQDTLKGRFTISRDNAKNTLYLQMNSLRAEDTAVYYCARGLSEGAWFAYWGQGTLVTVSSGGGGSDIVMTQTPLSLPVTLGQPASISCRSSQNIVHTNGNTYLEWYLQKPGQSPQLLIYKVSNRFSGVPDRFSGSGSGTDFTLKISRVEAEDVGVYYCFQGSHVPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 344 aa, synthetic construct
  REPEAT 120..124: note = 1, or 2, 3, 4, 5, 6, 7, 8, 9, 10, or more repeats
EVQLVESGGGLVQPGGSLRLSCATSGFTFSDYYIYWVRQAPGKGLEWVAYISQDGDITDYQDTLKGRFTISRDNAKNTLYLQMNSLRAEDTAVYYCARGLSEGAWFAYWGQGTLVTVSSGGGGSXDIVMTQTPLSLPVTLGQPASISCRSSQNIVHTNGNTYLEWYLQKPGQSPQLLIYKVSNRFSGVPDRFSGSGSGTDFTLKISRVEAEDVGVYYCFQGSHVPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 357 bp, synthetic construct
  source 1..357: mol_type = other DNA
gaggtgcagctggttgaatctggcggaggattggttcagcctggcggctctctgagactgtcttgtgccacctctggcttcaccttctccgactactacatctactgggtccgacaggcccctggcaaaggacttgagtgggtggcctacatcagccaggacggcgacatcaccgactaccaggataccctgaagggcagattcaccatcagccgggacaacgccaagaacaccctgtacctgcagatgaactccctgagagccgaggacaccgccgtgtactattgtgctagaggcctgtccgaaggcgcttggtttgcttattggggccagggcacactggtcaccgtgtcctct